(19) United States
(12) Patent Application Publication
(10) Pub. No.: US 2009/0233840 A1
(43) Pub. Date: Sep. 17, 2009

(54) Modified Dynorphin Expression in Animals and Identification of Compounds for Treatment of Obesity and Diabetes

(76) Inventors: Herbert Herzog, New South Wales (AU); Amanda Sainsbury-Salis, New South Wales (AU)

Correspondence Address: FENWICK & WEST LLP, SILICON VALLEY CENTER, 801 CALIFORNIA STREET, MOUNTAIN VIEW, CA, 94041, US

(21) Appl. No.: 11/573,681

(22) PCT Filed: Aug. 15, 2005
(86) PCT No.: PCT/AU05/01223

§ 371 Date: Sep. 18, 2007

(30) Foreign Application Priority Data
Aug. 13, 2004 (AU) 2004904586

(51) Int. Cl. A61K 38/02; A01K 67/027; C12N 15/85; G01N 33/50; C12N 5/10; C12Q 1/68; C12N 15/63; A61P 3/04

(52) U.S. Cl. 514/2; 800/13; 800/21; 800/3; 435/325; 435/6; 435/320.1

(57) ABSTRACT

The present invention provides novel transgenic animal models of reduced dynorphin expression in humans that are useful for identifying compounds that are antagonists, inverse agonists, agonists or mimetics of one or more dynorphin peptide products of preprodynorphin. The compounds identified in such screening assays are useful in contexts related to the treatment of obesity and type II diabetes including, for example, improving weight loss and/or reducing appetite during dieting; treating feeding disorders; and for modifying fat mass and reducing glucose intolerance in males.

### FIELD OF THE INVENTION

The present invention relates generally to transgenic animals having a reduced level of expression of one or more opioid peptides and uses therefore to identify compounds that antagonists, inverse agonists, agonists or mimetics of opioid peptides in humans and other animals. More particularly, this invention provides novel transgenic animal models of reduced dynorphin expression in humans that are useful for identifying compounds that are antagonists, inverse agonists, agonists or mimetics of one or more dynorphin peptide products of preprodynorphin. The compounds identified in such screening assays are useful in many contexts including, for example, improving weight loss and/or reducing appetite during dieting; treating feeding disorders; treating disorders of insulin clearance or metabolism, such as, for example, diabetes, obesity, anorexia or bulimia; modifying adiposity (including contents of white and brown adipose tissue); modifying liver function; modifying serum glucose tolerance; and modifying feeding behavior (e.g. in connection with treating overeating, bulimia or anorexia). The invention also relates to method for identifying modulators of insulin clearance, insulin resistance or glucose intolerance or metabolism that are useful in the therapeutic methods described herein, e.g. using a non-human animal model.

### BACKGROUND TO THE INVENTION

1. General

This specification contains nucleotide and amino acid sequence information prepared using Patentin Version 3.3. Each nucleotide sequence is identified in the sequence listing by the numeric indicator <210> followed by the sequence identifier (e.g. <210>1, <210>2, <213> etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (eg. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

The term “insulin resistance” as used herein shall be taken to mean a state in which an animal body does not respond to the action of insulin, although enough insulin is produced to reduce blood or serum glucose in a healthy subject, e.g., as often occurs in people with type II diabetes.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purposes of exemplification only. Functionally equivalent products, compositions and methods are clearly within the scope of the invention, as described herein.

The present invention is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in the following texts that are incorporated by reference:
1. Sambrook, Fritsch & Maniatis, Molecular Cloning: A Laboratory Manual, Cold Spring Harbor Laboratories, New York, Second Edition (1989), whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp1-22; Atkinson et al., pp35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;
5. Animal Cell Culture: Practical Approach, Third Edition (John R. W. Masters, ed., 2000), ISBN 0199637970, whole of text;
6. Immobilized Cells and Enzymes: A Practical Approach (1986) IRL Press, Oxford, whole of text;
7. Perbal, B., A Practical Guide to Molecular Cloning (1984);
8. Methods In Enzymology (S. Colowick and N. Kaplan, eds., Academic Press, Inc.), whole of series;
9. J. F. Ramalho Ortigão, “The Chemistry of Peptide Synthesis” In: Knowledge database of Access to Virtual Laboratory website (Interactiva, Germany);
10. Sakakibara, D., Teichman, J., Lien, E. Land Fenichel, R. L. (1976). Biochem. Biophys. Res. Commun. 73 336-342
11. Merrifield, R. B. (1963). J. Am. Chem. Soc. 85, 2149-2154.
12. Barany, G. and Merrifield, R. B. (1979) in The Peptides (Gross, E. and Meienhofer, J. eds.), vol. 2, pp. 1-284, Academic Press, New York.
13. Wünsch, E., ed. (1974) Synthese von Peptiden in Houben-Weyls Metoden der Organischen Chemie (Müler, E., ed.), vol.15, 4th edn., Parts 1 and 2, Thieme, Stuttgart.
14. Bodanszky, M. (1984) Principles of Peptide Synthesis, Springer-Verlag, Heidelberg.
15. Bodanszky, M. & Bodanszky, A. (1984) The Practice of Peptide Synthesis, Springer-Verlag, Heidelberg.
16. Bodanszky, M. (1985) Int. J. Peptide Protein Res. 25, 449-474.
17. Handbook of Experimental Immunology, Vols. I-IV (D. M. Weir and C. C. Blackwell, eds., 1986, Blackwell Scientific Publications).

2. Description of the Related Art

There are three traditional gene-related opioid peptides: proopiomelanocortin, proenkephalin, and prodynorphin.

The preprodynorphin gene encodes the [Leu]-enkephalin-containing peptides α-neo-endorphin, β-neo-endorphin, dynorphin A and dynorphin B which are absolutely conserved between humans and mice (FIG. 1).

A major projection system for dynorphin peptides in the magnocellular and parvocellular components of the hypothalamic paraventicular nucleus (PVN) projects to limbic, mesencephalic, pontine and medullary structures. Dynorphins are also found in the interneurons of many of these structures.

There are three opioid receptor subtypes, Mu (μ), kappa (κ) and delta (δ). Attempts to associate dynorphin expression with specific opioid receptors indicates that dynorphins bind to multiple opioid receptors (reviewed by Pasternak, Clin. Neuropharmacol 16, 1-18, 1993). The mu (μ), kappa (κ) and delta (δ) opioid receptor subtypes bind more than one endogenous opioid peptide. The dynorphin peptides have very high affinity for all three receptor subtypes.

The μ receptor has been further divided into μ1 and μ2 subtypes (Pasternak Life Sci. 38, 1889-1896, 1986). The μ1 receptor binds opiates and most enkephalins with similar high affinities and has been characterised using the irreversible μ1-selective antagonist, naloxonazine. The μ2 site is the prototypical site for morphine binding. Mu-selective drugs with activity at both μ1 and μ2 binding sites, include irreversible (β-funaltrexamine: [(βFNA]) and reversible (Cys2-Tyr3-Orn5-Pen7-amide [CTOP]) antagonists. Equally sensitive functional responses for βFNA and naloxonazine suggest μ1 receptor mediation, while selective sensitivity to βFNA, but not naloxonazine suggests μ2 receptor mediation.

The δ receptor has been subdivided into δ1 and δ2 subtypes based upon the δ1 actions of agonists (D-Pen², D-Pen5-enkephalin [DPDPE]) and antagonists (D-Ala², Leu5, Cys6-enkephalin [DALCE]), and upon the δ2 actions of agonists (D-Ala², Glu4-deltorphin [Delt II]) and antagonists (naltrindole isothiocyanate NTII]). Delta1 and δ2 opioid subtypes have been dissociated from each other in analgesic assays (Jiang et al. J. Phamacol. Exp. Ther. 257, 1069-1075, 1991; Mattia et al., J. Phamacol. Exp. Ther. 258, 583-587, 1991).

At least four different κ receptor subtypes have been characterised biochemically (Rothman et al., Peptides 11, 311-331, 1990, but the κ1 and κ3 opioid receptor subtypes have been characterised pharmacologically, using selective κ1 agonists (e.g., U50488H) and antagonists (nor-binaltorphamine [Nor-BNI]), and κ3 agonists (naloxone benzoylhydrazone [Na1BZOH]) as described by Paul et al., J. PhamacoL Exp. Ther. 255, 769-774, 1990.

Zhang et al., J. Pharmacol. Exp Ther. 286, 136-141, 1998 showed that, in addition to being a high affinity ligand for the human KOR-1 receptor with sub-nanomolar affinity, dynorphin A also binds to the human MOR-1 and DOR-1 receptors with affinity values in the nanomolar range. Zhang et al., (1998) also showed that shorter dynorphin fragments, such as dynorphin A-(1-8), have relatively higher affinity for MOR-1 and DOR-1 receptors compared to dynorphin A, suggesting that the higher affinity for the KOR-1 receptor may be contributed by the C-terminal portion of the dynorphin A peptide. Studies on the binding of dynorphins to cells transfected with the MOR-1 gene, DOR-1 gene, or KOR-1 gene, encoding μ-receptor, δ-receptor, and κ-receptor respectively, further indicate that the [Leu]-enkephalin core present in both dynorphin A and dynorphin B (i.e., Dyn1-5) is necessary and sufficient for binding to the μ and δ-receptors.

Transfected Xenopus oocytes co-expressing the hMOR-1, hDOR-1 or hKOR-1 receptor subtypes with a GIRK1 potassium channel are activated by dynorphin A to produce an inwardly-rectifying K+ current in a dose-dependent manner, demonstrating that dynorphin A binds and activates all three opioid receptors. The calculated EC₅₀ values for hMOR-1, hDOR-1 and hKOR-1 are 30±5, 84±11 and 0.43±0.08 nM (mean ±S.E., n=2-3), respectively, indicating that dynorphin A is a highly-potent agonist of the KOR-1 receptor, however also possesses agonist activity with respect to the MOR-1 and DOR-1 receptor subtypes (Zhang et al., J. Pharmacol. Exp Ther. 286,136-141, 1998).

Dynorphin has been reported to be involved in analgesia, however, reports concerning the analgesic efficacy of Dynorphin A (Dyn A) have been peculiar. Unlike other opioid peptides, Dyn A and related peptides administered ICV (intracerebroventricularly) have been found to be ineffective in thermal analgesic assays using heat, such as rat tail flick or hot plate tests. However, in non-thermal and mechanical analgesic assays, such as flinch-jump response to foot shock, the paw pressure test or the tail pinch test, these peptides have been reported to have a moderate analgesic effect intracerebroventricularly. Reports of spinal administration have also been conflicting. In the writhing assay Dyn A(1-13) and other smaller fragments have been found to be very potent and were not affected by the presence of naloxone or nor-Binaltorphimine (norBNI). This indicates that the spinal analgesia is mediated by non-opioid receptors. The analgesia resulting from spinal or supraspinal administration of Dyn A(1-13) is accompanied by non-opioid effects (motor disturbances, spinal cord injury and hind limb paralysis) that impair the assessment of its antinociceptive activity. The motor effects of Dyn A were attributed to a possible facilitation of excitatory amino acid activity. The involvement of NMDA receptor rather than opioid receptors in the motor effects of Dyn A(1-13) was accompanied by local ischemia, a phenomenon known to induce the secretion of excitatory amino acids. Despite these conflicting findings, Dyn A(1-13) and Dyn A(1-10) amide have been used successfully for the treatment of intractable pain in cancer patients.

It is likely that dynorphin A also has an opioid receptor-mediated inhibitory effect on calcium conductances in neuronal cells, because dynorphin A inhibits high-threshold, voltage-dependent calcium currents in sensory neurons via κ- and μ-receptors (Moises et al., J. Neurosci. 14, 5903-5916, 1994), and attenuates calcium channel-dependent synaptic transmission at mossy fibers in the hippocampal CA3 region (Castillo et al., J. Neurosci. 16, 5942-5950, 1996), and reduces calcium currents in acutely dissociated nodose ganglion neurons via a Gi/Go-coupled system in a naloxone-sensitive manner (Gross et al., Proc. Natl Acad. Sci. USA 87, 7015-7029, 1990). These effects are consistent with an inhibitory role of opioid receptors in neurotransmission and neurotransmitter release.

On the other hand, Tang et al., J. Neurophysiol. 83, 2610-2612, 2000 showed that dynorphin A (2-17) also produces an increase in intracellular calcium concentration in cultured cortical cells from Sprague Dawley rats comparable to that induced by N-methyl-D-aspartate (NMDA), suggesting that it may have a direct effect on neuronal cell excitability. However, this effect may not be opiate receptor-mediated, because the effect is resistant to the opioid antagonist naloxone (Tang et al., 2000). This excitatory action of dynorphin A may be associated with elevated levels of dynorphin A in vivo, such as that associated with injury to the nervous system.

Dynorphin has also been implicated in tumor formation (Smith and Lee Ann. Rev. Pharmacol. Toxicol., 28, 123-140,1988).

There is abundant evidence that obesity are increasing in the western populations. Surveys conducted during the 1990s showed that obesity, defined as being at least 30 percent above ideal body weight, increased across the United States from 12 percent of the population in 1991 to almost 18 percent in 1998. The latest data for the first quarter of 2002 show that more than 24 percent of American adults aged 20 and older are obese. The US Food and Drug Administration says about 300,000 U.S. deaths a year are associated with obesity and overweight. The FDA also says the total direct and indirect costs attributed to these conditions amounted to $117 billion in 2000.

Loss of as little as 5-10% of body weight by lifestyle intervention can significantly reduce or reverse the prevalence and severity of obesity-associated co-morbidities. However, for the millions of obese individuals worldwide who are overweight or obese, the long-term prognosis for weight loss is poor. Ninety five percent of people who lose excess weight by lifestyle interventions gain back all the weight they lost within 2 years. One of the major physiological obstacles to permanent weight reduction is that weight loss activates starvation defenses that stimulate appetite and reduce energy expenditure. Thus, obese subjects generally do not sustain weight loss or fat reduction for prolonged periods despite rapid initial weight loss. Additionally, appetite tends to increase during prolonged dieting making long-term adherence to a dietary regimen difficult. Hormonal and metabolic effects of the starvation response may contribute to the increase in appetite that inhibits weight loss during obesity treatments, however this is not proven. Accordingly, there is a need for drugs that can suppress appetite during diet and/or maintain weight loss during diet, to aid in reducing the incidence of obesity and/or improve the management of obesity in populations.

Obesity is a major risk factor for type II diabetes and other chronic diseases. Because adiposity is related to insulin resistance, and insulin resistance is a risk factor for type II diabetes and cardiovascular disease, a consensus has emerged that increased adiposity is responsible for the increased incidence of type II diabetes and its associated morbidity (metabolic syndrome or syndrome X). Evidence has also accumulated indicating that visceral adiposity in particular is associated with insulin resistance and the metabolic syndrome.

Despite overwhelming evidence demonstrating association between insulin resistance, visceral adiposity, and metabolic risk, there is little evidence directly demonstrating that central adiposity in fact causes insulin resistance. In addition, there is little understanding of the mechanisms underlying the relationship among visceral adiposity, insulin resistance, and risk.

A challenge for obesity research is the determination of the hypothalamic regulators of energy homeostasis and their interactions. In particular, the identification of key mediators of the starvation response, including those operating in additive pathways, is also important for identifying dual or multiple targets for novel weight-reducing pharmaceuticals.

There are insufficient experimental data available from established in vitro or in situ assay systems of opioid receptor activity to definitively demonstrate the role of endogenous opioids in energy homeostasis in vivo. For example, known opioid agonists and antagonists such as naloxone or naltrexone lack the specificity and long-term effectiveness required to determine the role of opioids in regulating obesity and the treatment thereof by dietary restriction. Similar limitations make it difficult to determine the role of opioids in the development of downstream complications associated with obesity e.g., type II diabetes and its complications. Known opioid agonists and antagonists such as naloxone or naltrexone also lack specificity and long-term effectiveness in the treatment of obesity. Novel pharmacological agents that can antagonize those cellular pathways involved in regulating appetite, feeding behaviour, body weight and adiposity are needed in order to improve the effectiveness of lifestyle interventions for weight loss and the treatment of obesity.

Animal models are highly desirable for determining those factors involved in mediating the long-term effects of fasting (a model of dieting) and obesity, and the long-term regulation of obesity and diet by factors such as hormones, neuropeptides and metabolic pathways. Such animals provide the means for a longitudinal assessment of the temporal changes in the factors that determine obesity, glucose intolerance and insulin resistance. By virtue of examining their phenotype, such models have utility in determining appropriate cellular targets for the treatment of obesity and for determining the efficacy or specificity of therapeutic compounds.

### SUMMARY OF THE INVENTION

In work leading up to the present invention, the inventors sought to determine the role of dynorphins in protecting against weight loss during food restriction. In preliminary studies, the inventors have shown that dynorphin mRNA is expressed in the hypothalamic arcuate nucleus and co-localized with expression of neuropeptide Y (NPY) (FIG. 2). The inventors also determined the effects of fasting, a model of dieting, on hypothalamic dynorphin expression, and observed increases in mRNA expression in wild-type mice that had been fasted (FIG. 4). The increase in pre-prodynorphin expression was in the arcuate nucleus, paraventricular nucleus, and ventro-medial hypothalamus (FIGS. 5a-5c). These data suggest a role for dynorphin in the regulation of energy homeostasis.

The inventors also generated pre-prodynorphin knockout mice that are deficient in all endogenous dynorphin opioids. In dynorphin-deficient mice, the expression of neuropeptide Y (NPY) mRNA is significantly decreased in the arcuate nucleus, indicating that dynorphins stimulate expression of NPY (FIG. 3). Reciprocally, the inventors showed that the increase in dynorphin expression observed in fasted animals does not occur in NPY-Y1 receptor knockout mice (FIG. 4; FIG. 5a-5c), indicating that NPY stimulates expression of dynorphins during fasting via NPY-Y1 receptors. Thus, a negative energy balance that arises during fasting may increase the expression of both dynorphins and NPY in the hypothalamus, by virtue of an auto-reinforcing feed-forward regulatory loop that stimulates appetite and hinders body weight loss.

Having established an interaction between NPY and dynorphin in regulating energy homeostasis during fasting, the inventors sought to establish the more detailed phenotype of the dynorphin knockout animals (FIGS. 7-20). The inventors showed that endogenous dynorphins mediate the appetite- and weight-promoting effects of food deprivation, since dynorphin knockout mice lose significantly more weight than wild type mice in response to a 24-hour fast (FIG. 9a, 9b), and eat significantly less food than wild type mice in the 24 hours post-fasting (FIG. 9c). Interestingly, these dynorphin-mediated effects on body weight and appetite may not be correlated with fat content of diet, since animals maintained on normal chow diets or high-fat diets, and without subsequent fasting, showed no difference in these parameters (FIGS. 14 and 15).

Relevant to obesity and its complications, targeted disruption of a pre-prodynorphin locus in mice sufficient to prevent functional preprodynorphin protein from being expressed, significantly decreases peripheral adiposity as determined by white adipose tissue (WAT) content, and brown adipose tissue (BAT) content (FIG. 10). Thus, dynorphins are involved in regulating fat mass. The effect on adiposity appears to be significant for animals on low-fat as opposed to high-fat diets, since wild-type and dynorphin-deficient animals maintained on high-fat diets show the same degree of adiposity (FIG. 16). Without being bound by any theory or mode of action, other metabolic or hormonal factors may mask the dynorphin-mediated reduction in adiposity for subjects on high-fat diets.

In male dynorphin knockout animals, the inventors found enhanced glucose tolerance in standard glucose tolerance tests (FIG. 12), indicating a role for dynorphins in mediating long-term glucose intolerance (i.e., dynorphins inhibit or reduce glucose uptake from serum). This suggests that dynorphins may be involved in the glucose intolerant phenotype observed in type II diabetic subjects. This is independent of any effect on basal serum insulin (FIG. 13).

The present inventors have also demonstrated that short-term starvation responses may be mediated by dynorphins, because IGF-1 levels are significantly reduced in chow-fed dynorphin knockout male animals following fasting (FIG. 17), however a reduction in serum IGF-1 following fasting is greater in both male and female dynorphin knockout animals than wild-type animals (FIGS. 17 and 18). Additionally, serum concentrations of free T4 are reduced significantly in female dynorphin knockout animals, and serum free T4 levels are reduced in a manner consistent with the effects of fasting on this hormone, however the reduction in serum free T4 following fasting of dynorphin knockout animals is greater than for wild-type animals (FIG. 20). These data suggest that dynorphin action is at least partially mediated by the hypothalamo-pituitary-thyrotropic and -somatotropic axes.

In summary, these data indicate that one or more dynorphin opioids are involved in regulating at least the following factors involved in obesity and its complications such as type II diabetes, and the management thereof:
(i) the modulation of starvation responses in mammals, in particular feeding behaviour and weight loss during dieting, fasting or short-term starvation;
(ii) adiposity e.g., fat deposition and/or fat mass, especially for animals that eat a diet that is not necessarily high in fat; and
(iii) in males, glucose intolerance.

These effects may further involve the action of neuropeptides such as, for example, NPY, and/or hormones, such as thyroixin or IGF-1. The preprodynorphin-deficient mouse model is therefore useful for identifying compounds that specifically modulate a preprodynorphin-mediated characteristic such as, for example, feeding behaviour (e.g. in the treatment of anorexia or bulimia), fat deposition, glucose uptake or insulin resistance. These modulators are identified by screening animals for their effect on the phenotype of the mouse model.

Accordingly, the present invention provides a transgenic animal in which the expression of one or more dynorphin opioid peptides is reduced or disrupted. As exemplified herein, targeted disruption of exons 3 and 4 of the preprodynorphin gene disrupts expression of all dynorphin peptides encoded by the preprodynorphin gene. The present invention clearly encompasses an animal in which the expression of any one or more dynorphin peptides is reduced or disrupted.

Preferably, the dynorphin-deficient animal of the invention exhibits one or more phenotypes distinct from wild-type animals, said phenotypes being selected from the group consisting of
1. Increased weight loss during dieting, fasting or short-term starvation;
2. Decreased appetite during dieting, fasting or short-term starvation;
3. Decreased adiposity, preferably when animals are maintained on a diet that is not high in fat;
4. Increased glucose tolerance in males;
5. Decreased NPY expression in the arcuate nucleus of the hypothalamus;
6. Reduced serum IGF-1 in fed and fasted males;
7. Enhanced reduction in serum IGF-1 following fasting;
8. Reduced serum free T4 in fed and fasted females; and
9. Enhanced reduction in free T4 in fasted females.

Preferably, the dynorphin-deficient animal of the invention exhibits at least increased weight loss and decreased food intake (i.e., decreased appetite) during dieting, fasting or short-term starvation compared to animals that express a functional pre-prodynorphin gene however are otherwise isogenic.

The present invention clearly encompasses dynorphin-deficient animals that comprise additional modification to their genomes, such as, for example, the knockout of an NPY-encoding gene, one or more NPY receptor-encoding genes (e.g., NPY-1, NPY-Y2, NPY-Y4, NPY-Y5, NPY-Y7 and combinations thereof) and/or a leptin-encoding gene. Particularly preferred dynorphin-deficient (i.e., dyn^{-/-}) animals of the present invention have a genotype selected from the group consisting of: (i) dyn^{-/-} NPY^{-/-}; (ii) dyn^{-/-} NPY-Y1^{-/-}; (iii) dyn^{-/-} NPY-Y2^{-/-}; (iv) dyn^{-/-} NPY-Y4^{-/-}; (v) dyn^{-/-} NPY-Y2-Y2^{-/-} NPY-Y4^{-/-}; and (vi) dyn^{-/-} leptin^{-/-} and combinations thereof.

It is also within the scope of the present invention for the dynorphin-deficient animal to comprise a genetic background that confers an obesity or diabetes syndrome on an animal having functional pre-prodynorphin expression. Thus, pre-prodynorphin gene expression is disrupted or knocked-out in an animal having an obesity or diabetes syndrome phenotype. Such animals will have the genetic background selected from the group consisting of:
(i) yellow obese (Ay/a), a dominant mutation causes the ectopic, ubiquitous expression of the agouti protein, resulting in a condition similar to adult-onset obesity and non-insulin-dependent diabetes mellitus (Michaud et al., Proc Natl Acad. Sci USA 91: 2562-2566,1994);
(ii) Obese (ob/ob) having a mutation in the gene encoding leptin (Zhang et al., Nature 372: 425-432,1994);
(iii) diabetes (db/db) having a mutation in the gene encoding the leptin receptor (Tartaglia et al., Cell 83: 1263-1271, 1995);
(iv) adipose (cpe/cpe) having a mutation in the gene encoding carboxypeptidase E (Naggert et al., Nat. Genet. 10: 135-142, 1995);
(v) tubby (tub/tub) having a mutation in a member of a new family of genes encoding tubby-like proteins (Kleyn et al., Cell 85: 281-290, 1996; Noben-Trauth et al., Nature 380: 534-548, 1996); and
(vi) combinations of any one or more of (i) to (v).

The present invention extends to any progeny of the dynorphin-deficient animal as described herein, including any animals that are heterozygous dyn^{+/+} or homozygous dyn^{-/-}, the only requirement being that they are deficient in at least one allele of a preprodynorphin-encoding gene. Preferably, such animals are produced by targeted disruption of a preprodynorphin-encoding gene such as, for example, by using the targeting construct comprising the structural feature set forth in FIG. 2 and/or comprising the nucleotide sequence set forth in SEQ ID NO: 7 or complementary thereto.

The present invention also provides a method of identifying a dynorphin-mediated phenotype, said method comprising comparing a phenotype of a wild-type animal to a phenotype of a genetically modified non-human animal comprising a genetic modification within an allele of its preprodynorphin locus wherein said genetic modification reduces or prevents expression of a functional dynorphin peptide in said animal, and wherein a modified or different phenotype is a dynorphin-mediated phenotype. Preferably, the method further comprising providing the genetically modified non-human animal. In this context, the term “providing” shall be taken to include producing the genetically modified non-human animal.

The present invention further provides a method of producing a dynorphin-deficient animal comprising introducing a vector comprising a nucleotide sequence set forth in SEQ ID NO: 7 or complementary thereto into the genome of a mammalian cell for a time and under conditions sufficient for homologous recombination to occur thereby deleting at least about exons 3 and 4 of a preprodynorphin-encoding gene from said genome and regenerating a whole animal there from.

The present invention also provides a vector comprising a nucleotide sequence set forth in SEQ ID NO: 7 or complementary thereto.

Based on the phenotype of the dynorphin-deficient animal of the present invention and the expression of dynorphin in the brain, the animal models of the present invention have particular utility for identifying compounds that modulate dynorphin expression or activity in vivo.

Accordingly, the present invention provides a method of identifying a compound that modulates a dynorphin-mediated phenotype of an animal, said method comprising: (a) administering a compound to a non-human animal expressing a functional dynorphin peptide and determining a dynorphin-mediated phenotype of the animal; and (b) comparing the dynorphin-mediated phenotype at (a) to the same phenotype of genetically modified non-human animal to which the compound has not been administered, said genetically modified non-human animal comprising a genetic modification within an allele of its preprodynorphin locus, wherein said genetic modification reduces or prevents expression of a functional dynorphin peptide in said animal, and wherein a comparable phenotype indicates that the compound modulates a dynorphin-mediated phenotype of the animal. Preferably, the dynorphin-mediated phenotype is selected from the group consisting of
1. Weight loss during dieting, fasting or short-term starvation;
2. Appetite during dieting, fasting or short-term starvation;
3. Adiposity, preferably when animals are maintained on a diet that is not high in fat;
4. Glucose tolerance in males;
5. NPY expression in the arcuate nucleus of the hypothalamus;
6. Serum IGF-1 level in fed and fasted males;
7. Rate of reduction in serum IGF-1 following fasting;
8. Serum free T4 level in fed and fasted females; and
9. Rate of reduction in free T4 in fasted females.

In one embodiment the compound is an antagonist or inverse agonist of a dynorphin peptide e.g., dynorphin A, dynorphin B or active fragment thereof [e.g., Dyn A (1-15), Dyn A (2-17), Dyn A (1-13), Dyn A (1-8), Dyn A (1-5), or other fragment], or alternatively, an antagonist or inverse agonist of one or more dynorphin receptors e.g., κ-opioid receptor and/or μ-opioid receptor and/or δ-opioid receptor. In accordance with this embodiment, the phenotype more closely resembles the phenotype of the genetically modified non-human animal comprising a genetic modification within an allele of its preprodynorphin locus to which the compound has not been administered (i.e., the phenotype of dynorphin-deficient animal of the present invention).

The present invention also provides a method of identifying a compound that modulates a dynorphin-mediated phenotype of an animal, said method comprising: (a) administering a compound to a genetically modified non-human animal comprising a genetic modification within an allele of its preprodynorphin locus wherein said genetic modification reduces or prevents expression of a functional dynorphin peptide in said animal; and (b) determining a phenotype of the animal relative to the phenotype of an isogenic animal to which the compound has not been administered wherein a different phenotype indicates that the compound modulates a dynorphin-mediated phenotype of the animal. Preferably, the dynorphin-mediated phenotype is selected from the group consisting of
1. Weight loss during dieting, fasting or short-term starvation;
2. Appetite during dieting, fasting or short-term starvation;
3. Adiposity, preferably when animals are maintained on a diet that is not high in fat;
4. Glucose tolerance in males;
5. NPY expression in the arcuate nucleus of the hypothalamus;
6. Serum IGF-1 level in fed and fasted males;
7. Rate of reduction in serum IGF-1 following fasting;
8. Serum free T4 level in fed and fasted females; and
9. Rate of reduction in free T4 in fasted females.

In one embodiment the compound is a dynorphin mimetic compound e.g., a mimetic or analogue of dynorphin A, dynorphin B or active fragment thereof [e.g., Dyn A (1-15), Dyn A (2-17), Dyn A (1-13), Dyn A (1-8), Dyn A (1-5), or other fragment], or alternatively, an agonist of one or more dynorphin receptors e.g., κ-opioid receptor and/or μ-opioid receptor and/or δ-opioid receptor. In accordance with this embodiment, the phenotype more closely resembles the phenotype of a wild-type animal to which the compound has not been administered.

In each of the screening assays supra the present invention clearly contemplates the additional step of formulating the identified compound in a suitable carrier or excipient for administration to an animal subject. Alternatively or in addition, the subject method preferably further comprises producing or synthesizing the compound that is tested on the genetically modified animal. Alternatively or in addition, the subject method preferably comprises determining the structure of the compound. Alternatively or in addition, the subject method preferably comprises providing the name or structure of the compound.

In another embodiment, the subject method further comprises providing the compound.

The present invention clearly contemplates additional screening steps to those specifically stated herein, such as, for example, for the purposes of conducting high throughput primary screens to identify candidate compounds for screening in vivo using the animal model provided herein, or alternatively, for validating or otherwise confirming the binding and/or activity modulation activity of a compound identified using the animal model provided herein e.g., with respect to agonism or antagonism of one or more opioid receptors by a dynorphin peptide. Accordingly, in an alternative embodiment the method of identifying a compound that modulates a dynorphin-mediated phenotype of an animal further comprises determining a compound that binds to a dynorphin receptor and/or reduces the dynorphin-mediated activation of a dynorphin receptor and/or activates a dynorphin receptor.

In a particularly preferred embodiment, the present invention provides a process for identifying a compound that modulates a dynorphin-mediated phenotype of an animal said process comprising: (a) determining a compound that binds to a dynorphin receptor and/or reduces the dynorphin-mediated activation of a dynorphin receptor and/or produces a dynorphin-mediated change in intracellular calcium concentration; (b) administering a compound to a non-human animal expressing a functional dynorphin peptide and determining a dynorphin-mediated phenotype of the animal; (c) comparing the dynorphin-mediated phenotype at (b) to the same phenotype of genetically modified non-human animal to which the compound has not been administered, said genetically modified non-human animal comprising a genetic modification within an allele of its preprodynorphin locus, wherein said genetic modification reduces or prevents expression of a functional dynorphin peptide in said animal, and wherein a comparable phenotype indicates that the compound modulates a dynorphin-mediated phenotype of the animal.

In an alternative embodiment, the present invention provides a process for identifying a compound that modulates a dynorphin-mediated phenotype of an animal said process comprising: (a) determining a compound that binds to a dynorphin receptor and/or activates a dynorphin receptor and/or produces a dynorphin-mediated change in intracellular calcium concentration; (b) administering a compound to a genetically modified non-human animal comprising a genetic modification within an allele of its preprodynorphin locus wherein said genetic modification reduces or prevents expression of a functional dynorphin peptide in said animal; and (c) determining a phenotype of the animal relative to the phenotype of an isogenic animal to which the compound has not been administered wherein a different phenotype indicates that the compound modulates a dynorphin-mediated phenotype of the animal.

To achieve high throughput, preferred methods for determining a compound that binds to a dynorphin receptor and/or reduces the dynorphin-mediated activation of a dynorphin receptor and/or activates a dynorphin receptor and/or produces a dynorphin-mediated change in intracellular calcium concentration are performed in vitro in isolated cells or in situ using isolated neuron preparations. For example, competition assays can be performed in which the binding of a dynorphin peptide to a κ-opioid receptor and/or μ-opioid receptor and/or δ-opioid receptor expressed in transfected Xenopus oocytes or isolated HN9.10 cells stably expressing the receptor(s) is determined in the presence and absence of the compound. The dynorphin peptide may be detectably-labeled with a suitable reporter molecule, e.g., using a radioisotope. Alternatively, or in addition, competition assays can be performed in which activation of one or more opioid receptors is determined by measuring dynorphin-evoked potassium (K⁺) currents in transfected Xenopus oocytes or isolated HN9.10 cells stably expressing a GIRK-1 channel and one or more of a κ-opioid receptor, a μ-opioid receptor and a δ-opioid receptor, in the presence and absence of the compound. Alternatively, or in addition, competition assays can be performed in which intracellular calcium concentration of cultured cortical neuronal cells, e.g., HN9.10 cells, is determined in the presence and absence of the compound.

More specific compound screens of the present invention will be apparent from the disclosure herein.

For example, the present invention also provides a method of identifying a compound that enhances feeding behaviour in a fasted animal, such as, for example, in the treatment of anorexia, said method comprising: (a) administering a compound to a genetically modified non-human animal, said animal comprising a genetic modification within an allele of its preprodynorphin locus wherein said genetic modification reduces or prevents expression of a functional dynorphin peptide in said animal; and (b) determining the feeding behaviour of the animal, wherein enhanced appetite or dietary intake of the animal compared to the appetite or dietary intake of a preprodynorphin-deficient animal to which the compound has not been administered indicates that the compound enhances feeding behaviour in a fasted animal. Preferably the animal has been fasted before the compound is administered, or alternatively, is fasted whilst the compound is being administered.

The present invention also provides a method of identifying a compound that reduces feeding behaviour during dieting or starvation, such as, for example, in the treatment of obesity or Type II diabetes, said method comprising: (a) administering a compound to a non-human animal expressing a functional dynorphin peptide; and (b) comparing the feeding behaviour of the animal to the feeding behaviour of a genetically modified non-human animal comprising a genetic modification within an allele of its preprodynorphin locus wherein said genetic modification reduces or prevents expression of a functional dynorphin peptide in said animal, wherein a comparable feeding behaviour indicates that the compound reduces feeding behaviour during dieting or starvation. Preferably the animal has been fasted before the compound is administered, or alternatively, is fasted whilst the compound is being administered.

The present invention also provides a method of identifying a compound that enhances adiposity such as, for example, in the treatment of hypolipidemia (e.g. as observed in subjects suffering from abetalipoproteinemia, malnutrition or hematologic malignancies, such as acute myelocytic leukemia or chronic myelocytic leukemia), said method comprising: (a) administering a compound to a genetically modified non-human animal comprising a genetic modification within an allele of its preprodynorphin locus wherein said genetic modification reduces or prevents expression of a functional dynorphin peptide in said animal; and (b) determining the fat content of the animal, wherein enhanced fat content of the animal compared to the fat content of a preprodynorphin-deficient animal to which the compound has not been administered indicates that the compound enhances adiposity.

The present invention provides a method of identifying a compound that reduces adiposity, such as, for example, in the treatment of obesity or neurodegenerative disorders or for cosmetic purposes such as bodybuilding or weight loss, said method comprising: (a) administering a compound that enhances fat deposition or glucose uptake to a genetically modified non-human animal comprising a genetic modification within an allele of its preprodynorphin locus wherein said genetic modification reduces or prevents expression of a functional dynorphin peptide in said animal and determining the fat content of the animal; (b) administering a test compound to the animal and determining the fat content of the animal, wherein a similar or reduced fat content at (b) compared to (a) indicates that the compound reduces fat deposition.

The present invention provides a method of identifying a compound that reduces adiposity comprising: (a) administering a compound to a non-human animal expressing a functional preprodynorphin protein and determining the fat content of the animal; and (b) comparing the fat content at (a) to the fat content of a genetically modified non-human animal comprising a genetic modification within an allele of its preprodynorphin locus wherein said genetic modification reduces or prevents expression of a functional dynorphin peptide in said animal, wherein a comparable fat content indicates that the compound reduces adiposity.

The present invention also provides a method of identifying a compound that enhances glucose tolerance as, for example, in the treatment of hyperglycemia or insulin resistance (e.g. as observed in subjects suffering from type II diabetes), said method comprising: (a) administering a compound to a genetically modified non-human male animal comprising a genetic modification within an allele of its preprodynorphin locus wherein said genetic modification reduces or prevents expression of a functional dynorphin peptide in said animal; and (b) performing a glucose tolerance test on the animal, wherein reduced serum glucose content of the animal compared to the serum glucose content of an isogenic male animal to which the compound has not been administered indicates that the compound enhances glucose tolerance.

The present invention also provides a method of treating obesity in a subject comprising administering to the subject a compound that antagonizes the action of a dynorphin peptide in the subject.

The present invention also provides a method of reducing appetite or food intake during dieting or fasting of a subject comprising administering to the subject a compound that antagonizes the action of a dynorphin peptide in the subject.

The present invention also provides a method of increasing the reduction in body weight during dieting or fasting of a subject comprising administering to the subject a compound that antagonizes the action of a dynorphin peptide in the animal.

The present invention also provides a method of reducing glucose intolerance of a male subject comprising administering to the subject a compound that antagonizes the action of a dynorphin peptide in the subject.

Additional embodiments of the present invention will be apparent from the following disclosure including the accompanying drawings and Sequence Listing.

### BRIEF DESCRIPTION OF THE DRAWINGS

FIG. 1 is a schematic representation showing a ClustalW alignment between the amino acid sequences of mouse (SEQ ID NOs: 1 and 4) and human (SEQ ID NO: 2) preprodynorphin polypeptides. The amino acid sequences of a-neo-endorphin (residues 166-175 of mouse preprodynorphin), dynorphin A (residues 202-218 of mouse preprodynorphin) and dynorphin B (residues 221-233 of mouse preprodynorphin) are underlined in both the human and mouse sequences. Residues in bold type are [Leu]-enkephalin. Data indicate that the mouse and human dynorphin peptides derived from preprodynorphin are absolutely conserved notwithstanding that there is about 60-65% identity between the preprodynorphins of mouse and humans.

FIG. 2 is a copy of a photographic representation showing co-expression of mRNA encoding dynorphin (Dyn) and neuropeptide Y (NPY) in the hypothalamic arcuate nucleus (Arc) of the mouse central nervous system (CNS). Tissue was probed with (i) digoxigenin-labelled antisense nucleic acid capable of specifically hybridizing to dynorphin-encoding mRNA (Dyn-dig) and (ii) ³⁵S-labelled antisense nucleic acid capable of specifically hybridizing to NPY-encoding mRNA (NPY-³⁵S). The Dyn-dig probe is detectable as large, grey stained patches. The NPY-³⁵S probe is detectable as discrete small dark spots. Arrows indicate those cells that co-express dynorphin and NPY. 3v indicates third cerebral ventricle.

FIG. 3a is a copy of a photographic representation showing expression of mRNA encoding neuropeptide Y (NPY) in the hypothalamic arcuate nucleus (Arc) of a wild-type mouse i.e., having functional preprodynorphin gene expression. Tissue was probed with ³⁵S-labelled antisense nucleic acid capable of specifically hybridizing to NPY-encoding mRNA i.e., NPY-³⁵S. The NPY-³⁵S probe is detectable as discrete small dark spots. Tissue was also probed with digoxigenin-labelled antisense nucleic acid capable of specifically hybridizing to dynorphin-encoding mRNA (Dyn-dig). The Dyn-dig probe is detectable as large, grey stained patches. 3v indicates third cerebral ventricle.

FIG. 3b is a copy of a photographic representation showing expression of mRNA encoding neuropeptide Y (NPY) in the hypothalamic arcuate nucleus (Arc) of a mouse in which both copies of a preprodynorphin gene have been disrupted by homologous recombination using the targeting vector shown in FIG. 6 (i.e., Dyn^{-/-}). Tissue was probed with ³⁵S-labelled antisense nucleic acid capable of specifically hybridizing to NPY-encoding mRNA i.e., NPY-³⁵S. The NPY-³⁵S probe is detectable as discrete small dark spots at much reduced intensity relative to that observed in wild-type mice (FIG. 3a). Tissue was also probed with digoxigenin-labelled antisense nucleic acid capable of specifically hybridizing to dynorphin-encoding mRNA (Dyn-dig) and, as expected, no Dyn-dig probe hybridization was detectable above background levels in the Dyn^{-/-} mouse. 3v indicates third cerebral ventricle.

FIG. 4 is a graphical representation showing levels of mRNA encoding preprodynorphin as a percentage of wild-type levels in wild type mice (wild-type), mice having both copies of the NPY-Y1 receptor-encoding gene disrupted by homologous recombination (Y1 knockout) and mice having both copies of the NPY-Y2 receptor-encoding gene disrupted by homologous recombination (Y2 knockout). Mice were either maintained on a normal chow diet (i.e., Fed; open bars) or alternatively, fasted for 24 hours prior to sampling (i.e., Fasted; filled bars). Data represent the mean ±SEM for n=5-6 mice per group. The asterisk indicates a significant difference between the fed and fasted groups (p<0/05).

FIG. 5a is a graphical representation showing levels of mRNA encoding preprodynorphin as a percentage of wild-type levels in the arcuate nuclei (Arc) of wild type mice (wild-type), mice having both copies of the NPY-Y1 receptor-encoding gene disrupted by homologous recombination (Y1 knockout) and mice having both copies of the NPY-Y2 receptor-encoding gene disrupted by homologous recombination (Y2 knockout). Mice were either maintained on a normal chow diet (i.e., Fed; open bars) or alternatively, fasted for 24 hours prior to sampling (i.e., Fasted; filled bars). Data represent the mean ±SEM for n=5-6 mice per group. The asterisk indicates a significant difference between the fed and fasted groups (p<0/05).

FIG. 5b is a graphical representation showing levels of mRNA encoding preprodynorphin as a percentage of wild-type levels in the paraventricular nuclei (PVN) of wild type mice (wild-type), mice having both copies of the NPY-Y1 receptor-encoding gene disrupted by homologous recombination (Y1 knockout) and mice having both copies of the NPY-Y2 receptor-encoding gene disrupted by homologous recombination (Y2 knockout). Mice were either maintained on a normal chow diet (i.e., Fed; open bars) or alternatively, fasted for 24 hours prior to sampling (i.e., Fasted; filled bars). Data represent the mean ±SEM for n=5-6 mice per group. The asterisk indicates a significant difference between the fed and fasted groups (p<0/05).

FIG. 5c is a graphical representation showing levels of mRNA encoding preprodynorphin as a percentage of wild-type levels in the ventromedial hypothalamus (VMH) of wild type mice (wild-type), mice having both copies of the NPY-Y1 receptor-encoding gene disrupted by homologous recombination (Y1 knockout) and mice having both copies of the NPY-Y2 receptor-encoding gene disrupted by homologous recombination (Y2 knockout). Mice were either maintained on a normal chow diet (i.e., Fed; open bars) or alternatively, fasted for 24 hours prior to sampling (i.e., Fasted; filled bars). Data represent the mean ±SEM for n=5-6 mice per group. The asterisk indicates a significant difference between the fed and fasted groups (p<0/05).

FIG. 6 is a schematic representation showing the alignment of FIGS. 6a, 6b and 6c that show the structure of a first portion of a Cre-lox targeting vector for disrupting expression of a preprodynorphin gene by means of homologous recombination. There is overlap between FIGS. 6b and 6c such that the loxP site (triangle) at the far right of FIG. 6b is reproduced at the left of FIG. 6c, however there is only a single loxP site in this region of the targeting construct.

FIG. 6a is a schematic representation showing the structure of a first portion of a Cre-lox targeting vector for disrupting expression of a preprodynorphin gene by means of homologous recombination. This portion of the targeting construct comprises the 5′-untranslated (UTR) of the preprodynorphin gene (5′ arm Dyn) to and including the translation start site of the preprodynorphin gene (ATG) placed immediately upstream of a tetracycline repressor (rTetR) such that expression of rTetR is capable of being placed operably under the control of the preprodynorphin promoter sequence following homologous recombination of the targeting construct with murine genomic DNA. T7, is a bacteriophage T7 promoter present in pBluescript (Stratagene) vector DNA. The lower row of FIG. 6a shows the length (kb) of the components of the targeting construct within this portion. Restriction enzyme sites for the enzymes ApaI, AatI, SphI, NcoI, XhoI, PstI, EcoRI (E), BamHI (B) and XbaI (XbI) are shown.

FIG. 6b is a schematic representation showing the structure of a second (middle) portion of a Cre-lox targeting vector for disrupting expression of a preprodynorphin gene by means of homologous recombination. This portion of the targeting construct comprises from left to right: (i) a first gene construct that comprises the neomycin phosphotransferase gene (Neo) and enhanced green fluorescent protein gene (EGFP) placed operably under the control of the same PGK promoter and upstream of an SV40 polyadenylation sequence (open box labeled SV40 polyA), wherein said first gene construct further comprises a loxP site (darker-shaded triangle) positioned between PGK-Neo and EGFP; (ii) a zeocin resistance gene (Zeo); and (iii) an SV40 polyadenylation sequence (open box marked SV40 polyA) and loxP site (darker shaded triangle) comprising the 3′-end of a second gene construct comprising a Cre recombinase-encoding gene placed operably under control of a promoter comprising tetracycline repressor element (TRE) and upstream of an SV40 polyadenylation sequence (SV40 polyA), wherein said second gene construct further comprises a loxP site (triangle) positioned between Cre and SV40 polyA. Arrow indicates the direction of transcription of the first gene construct (i.e., right to left in the drawing). The lower row of FIG. 6b shows the length (kb) of the components of the targeting construct within this portion. Restriction enzyme sites for the enzymes PacI, BglII, SalI, HindIII, DralI, SpeI, EcoRI, BamHI (B) and XbaI are shown.

FIG. 6c is a schematic representation showing the structure of a third portion of a Cre-lox targeting vector for disrupting expression of a preprodynorphin gene by means of homologous recombination. This portion of the targeting construct comprises from left to right: (i) the 5′-end of the second gene construct described in the legend to FIG. 6b comprising the Cre recombinase-encoding gene and its endogenous promoter (Cre), the tetracycline repressor element (TRE) and loxP site (triangle); (ii) the 3′-arm of the targeting vector comprising the 3′-end of the preprodynorphin-encoding gene and downstream mouse genomic DNA (3′arm) cloned as an NdeI-EcoRI fragment adjacent to the TRE; and (iii) the bacteriophage SP6 promoter of the pBluescript (Stratagene) vector. The lower row of FIG. 6c shows the length (kb) of the components of the targeting construct within this portion. Restriction enzyme sites for the enzymes BamHI (B), Sacl, SaclI, EcoRI, EcoRV, ClaI, DralI, MluI, NdeI, KpnI, XbaI, NsiI, BstXI and NotI are shown.

FIG. 7 is a graphical representation showing that targeted disruption of preprodynorphin gene expression has no measurable effect on body weight of subjects maintained on a diet that is not high in fat (a chow diet). Data show mean body weights (g) ±SEM of groups of 25 wild-type mice (filled squares) and 25 dynorphin knockout mice (filled diamonds) during the first 26 weeks of life.

FIG. 8 is a graphical representation showing that targeted disruption of preprodynorphin gene expression has no measurable effect on basal food intake (g/24 hours) of male and female subjects maintained on a diet that is not high in fat (a chow diet). Data show mean food intake (g/24 hours) ±SEM of groups of 5-7 wild-type mice (open bars) and 5-7 dynorphin knockout mice (filled bars).

FIG. 9a is a graphical representation showing that targeted disruption of preprodynorphin gene expression increases weight loss during fasting of subjects. Data show mean body weights (g) ±SEM of groups of 10-14 wild-type mice (open bars) and 10-14 dynorphin knockout mice (filled bars) following 24 hours of fasting. Asterisks indicate that data are significantly different for wild-type and dynorphin knockout mice (p<0.01).

FIG. 9b is a graphical representation showing that targeted disruption of preprodynorphin gene expression increases the percentage weight loss during fasting of subjects. Data show mean percent weight lost ±SEM of groups of 10-14 wild-type mice (open bars) and 10-14 dynorphin knockout mice (filled bars) following 24 hours of fasting. Asterisks indicate that data are significantly different for wild-type and dynorphin knockout mice (p<0.01).

FIG. 9c is a graphical representation showing that targeted disruption of preprodynorphin gene expression decreases appetites during fasting of subjects as determined by the amount of food eaten following a 24 hour fast. Data show mean food eaten in the first hour post fasting (g) ±SEM of groups of 10-14 wild-type mice (open bars) and 10-14 dynorphin knockout mice (filled bars). Asterisks indicate that data are significantly different for wild-type and dynorphin knockout mice (p<0.01).

FIG. 10 is a graphical representation showing that targeted disruption of preprodynorphin gene expression decreases fat mass of subjects. Data show the mean weight of white adipose tissue (WATi, WATe, WATr, WATm, WATt) and brown adipose tissue (BAT) expressed as a percentage of body weight (% BWT) ±SEM of groups of 19-20 wild-type mice (open bars) and 19-20 dynorphin knockout mice (filled bars). WATi, WAT obtained from right inguinal tissue; WATe, WAT obtained from right epididymal tissue of males or periovarian (gonadal) tissue of females; WATr, WAT obtained from right retroperitoneal tissue; WATm, WAT obtained from mesenteric tissue; WATt, total WAT. Single asterisk indicates that data are significantly different for wild-type and dynorphin knockout mice (p<0.05). Double asterisks indicate that data are significantly different for wild-type and dynorphin knockout mice (p<0.01). Triple asterisks indicate that data are significantly different for wild-type and dynorphin knockout mice (p<0.001).

FIG. 11 is a graphical representation showing that targeted disruption of preprodynorphin gene expression has no measurable effect on diurnal activity of subjects. Data show mean movements (counts) per 30 minute ±SEM of groups of 6 wild-type mice (squares) and 6 dynorphin knockout mice (circles) during a 36 hour time period. The dark phase of the diurnal cycle is indicated by the heavy line on the x-axis between 18 and 30 hours.

FIG. 12 is a graphical representation showing that targeted disruption of preprodynorphin gene expression enhances glucose tolerance in male subjects in a standard glucose tolerance test. Data show mean serum glucose levels (mM) ±SEM for 2 hours (120 min) following commencement of the tolerance test. Groups of 8-10 wild-type male mice (diamonds) and 8-10 dynorphin knockout male mice (squares) were tested.

FIG. 13 is a graphical representation showing that targeted disruption of preprodynorphin gene expression has no measurable effect on basal serum insulin levels in subjects. Data show mean serum insulin concentration (pM) ±SEM of groups of 8-10 wild-type mice (open bar) and 8-10 dynorphin knockout mice (filled bar; Dyn^{-/-}). Similar data were obtained for fed and fasted animals.

FIG. 14 is a graphical representation showing that targeted disruption of preprodynorphin gene expression has no measurable effect on body weight of subjects maintained on a diet that is not high in fat (Normal Chow) or a high fat diet, i.e., without fasting. Data show mean body weight (g) ±SEM of groups of 14-20 wild-type mice (open bars) and 14-20 dynorphin knockout mice (filled bars).

FIG. 15 is a graphical representation showing that targeted disruption of preprodynorphin gene expression has no measurable effect on food intake of subjects maintained on a diet that is not high in fat (Normal Chow) or a high fat diet, i.e., without fasting. Data show mean food intake (g/24 hours) ±SEM of groups of 14-20 wild-type mice (open bars) and 14-20 dynorphin knockout mice (filled bars).

FIG. 16 is a graphical representation showing that targeted disruption of preprodynorphin gene expression reduces total fat mass of subjects maintained on low fat diets (Normal Chow) however has no measurable effect on total fat mass of subjects maintained on a diet that is high in fat (High Fat Diet). Data show mean total adipose tissue weight expressed as a percentage of body weight (% BWT) ±SEM of groups of 14-20 wild-type mice (open bars) and 14-20 dynorphin knockout mice (filled bars). Double asterisks indicate that data are significantly different for wild-type and dynorphin knockout mice (p<0.01).

FIG. 17 is a graphical representation showing that targeted disruption of preprodynorphin gene expression reduces serum IGF-1 levels in male subjects in the fed state, and enhances the reduction in serum IGF-1 following fasting. Data show the mean IGF-1 content per cell ±SEM of (i) wild-type chow-fed young male mice (WT C Y M); (ii) dynorphin knockout chow-fed young male mice (Dyn C Y M); (iii) wild-type young male mice maintained on a chow diet and then fasted (WT C Y M Fasted); and (iv) dynorphin knockout young male mice maintained on a chow diet and then fasted (Dyn C Y M Fasted).

FIG. 18 is a graphical representation showing that targeted disruption of preprodynorphin gene expression has no significant effect on serum IGF-1 levels in female subjects in the fed state, however enhances the reduction in serum IGF-1 of females following fasting. Data show the mean IGF-1 content per cell ±SEM of (i) wild-type chow-fed young female mice (WT C Y F); (ii) dynorphin knockout chow-fed young female mice (Dyn C Y F); (iii) wild-type young female mice maintained on a chow diet and then fasted (WT C Y F Fasted); and (iv) dynorphin knockout young female mice maintained on a chow diet and then fasted (Dyn C Y F Fasted).

FIG. 19 is a graphical representation showing that targeted disruption of preprodynorphin gene expression has no significant effect on serum thyroid hormone (T4) levels in male subjects in the fed state or following fasting. Data show the mean T4 content per cell ±SEM of (i) wild-type chow-fed male mice (WT C Y M); (ii) dynorphin knockout chow-fed male mice (Dyn C Y M); (iii) wild-type male mice maintained on a chow diet and then fasted (WT C Y M Fasted); and (iv) dynorphin knockout male mice maintained on a chow diet and then fasted (Dyn C Y M Fasted).

FIG. 20 is a graphical representation showing that targeted disruption of preprodynorphin gene expression reduces serum thyroid hormone (T4) levels in female subjects and enhances the reduction in serum T4 following fasting of female subjects. Data show the mean T4 content per cell ±SEM of (i) wild-type chow-fed female mice (WT C Y F); (ii) dynorphin knockout chow-fed female mice (Dyn C Y F); (iii) wild-type female mice maintained on a chow diet and then fasted (WT C Y F Fasted); and (iv) dynorphin knockout female mice maintained on a chow diet and then fasted (Dyn C Y F Fasted).

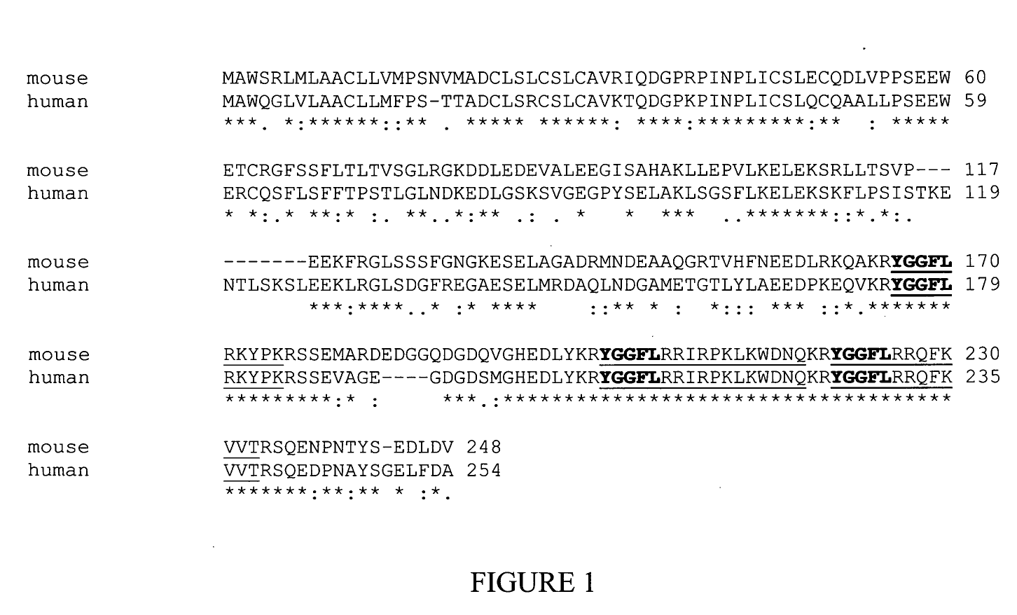

### DETAILED DESCRIPTION OF THE PREFERRED EMBODIMENTS

### Definition of Preprodynorphin Protein

As used herein, the term “preprodynorphin” shall be taken to mean any peptide, polypeptide, or protein having at least about 65% amino acid sequence identity to the amino acid sequence of a human or mouse preprodynorphin polypeptide set forth in SEQ ID NO: 1 or 2.

The term “preprodynorphin” shall also be taken to include a peptide, polypeptide or protein having the known biological activity of preprodynorphin, or the known binding specificity of preprodynorphin including c-preprodynorphin.

For the purposes of nomenclature, the amino acid sequences of the murine and human preprodynorphin polypeptides are exemplified herein, as SEQ ID NOs: 1 and 2, respectively. Preferably, the percentage identity to SEQ ID NO: 1 or 2 is at least about 70%, or 80% or 85%, more preferably at least about 90%, even more preferably at least about 95% and still more preferably at least about 99%.

The term “dynorphin” shall be taken to mean any active opioid peptide that is processed from a preprodynorphin polypeptide.

### Transgenic Animal Model

As exemplified herein, the present inventors have produced a mouse in which the expression of preprodynorphin is prevented, by gene targeting means.

In particular, the present invention provides a genetically modified non-human mammal which lacks a functional dynorphin gene referred to herein as a “non-human dynorphin knockout mammal” or a “dynorphin knockout mammal”. For example, the genome of the dynorphin knockout mammal comprises at least one non-functional allele for the endogenous dynorphin gene. Accordingly, the invention provides a source of a cell, a tissue, a cellular extract, an organelle or an animal useful for elucidating the function of dynorphin in an intact animal whose genomes comprise one copy of a wild-type dynorphin gene or no copies of a functional dynorphin gene. Furthermore, the invention provides a source of a cell, a tissue, a cellular extract, an organelle or an animal useful for determining a mimetic of dynorphin.

As the structure and function of dynorphin and derivatives thereof is conserved between mammalian species (e.g., between mouse and human) the effect of reducing the expression of dynorphin or a derivative thereof in a non-human mammal, e.g., a mouse, is a model system for the effect/s of such silencing in a human. Furthermore, a compound that is a mimetic of dynorphin determined using such a model organism is also considered an attractive target for treating a disease or disorder associated with reduced dynorphin expression.

Any suitable mammal can be used to produce the dynorphin knockout mammal described herein. For example, a suitable mammal can be, a mouse, a rat, a rabbit, a pig, a sheep or a cow. As exemplified herein, a mouse is suitable for production of a dynorphin knockout mammal.

As used herein the terms “non-human dynorphin knockout mammal” and “dynorphin knockout mammal” refer to a non-human mammal that comprises a genome comprising a disrupted or inactivated dynorphin gene. Those of skill in the art will recognize that the term “knockout” refers to the functional inactivation of the gene. The disruption introduces a chromosomal defect (e.g., mutation or alteration) in the dynorphin gene at a point in the nucleic acid sequence that is necessary for either the expression of the dynorphin gene or the production of a functional dynorphin protein. Accordingly, the introduction of the disruption inactivates the endogenous target gene (e.g., dynorphin gene).

As used herein the terms “disruption” and “functional inactivation” shall be taken to mean a partial or complete reduction in the expression and/or function of the dynorphin polypeptide encoded by the endogenous gene of a single type of cell, selected cells or all of the cells of a non-human transgenic dynorphin knockout animal. For example, a disrupted dynorphin gene is unable to encode pre- pro-dynorphin in one or more cells of an animal subject. Alternatively, or in addition, a disrupted dynorphin gene is usable to produce a derivative of dynorphin, such as, for example, a derivative selected from the group consisting of, Dynorphin A, DYN-(1-5), DYN-(1-8), DYN-(1-13), dynorphin-B, α-neo-endorphin β-neo-endorphin and mixtures thereof. Thus, according to the instant invention the expression or function of a dynorphin gene product (or derivative thereof) can be completely or partially disrupted or reduced (e.g., by 50%, 75%, 80%, 90%, 95% or more) in a selected group of cells (e.g., a tissue or organ) or in the entire animal. As used herein the term “a functionally disrupted dynorphin gene” includes a modified dynorphin gene which either fails to express any polypeptide product or which expresses a truncated protein having less than the entire amino acid polypeptide chain of a wild-type protein or a derivative thereof and is non-functional (partially or completely non-functional).

Disruption of the dynorphin gene or a region or derivative thereof is accomplished by any of a variety of methods known to those of skill in the art. For example, gene targeting using homologous recombination.

For example, the invention provides a knockout mammal whose genome comprises either a homozygous or heterozygous disruption of its dynorphin gene or a region thereof. A knockout mammal whose genome comprises a homozygous disruption is characterized by somatic and germ cells which contain two non-functional (disrupted) alleles of the dynorphin gene while a knockout mutant whose genome comprises a heterologous disruption is characterized by somatic and germ cells which contain one wild-type allele and one non-functional allele of the dynorphin gene.

As used herein, the term “genotype” refers to the genetic makeup of an animal with respect to the dynorphin chromosomal locus. More specifically the term genotype refers to the status of the animal's dynorphin alleles, which can either be intact (e.g., wild-type or +/+); or disrupted (e.g., knockout) in a manner which confers either a heterozygous (e.g., +/−); or homozygous (−/−) knockout genotype.

The present invention also provides a method for producing a non-human mammal which lacks a functional dynorphin gene. Methods for producing a “knockout mammal” are known in the art and described, for example, in Nagy et al eds. Manipulating the Mouse Embryo, Cold Spring Harbor Laboratory, 3rd Edition, 2002, ISBN 0879695749 and Tymms and Kola eds Gene Knockout Protocols, Humana Press, 2001, ISBN: 0896035727.

To produce a mutant mouse strain by homologous recombination, two major elements are generally used. An embryonic stem (ES) cell line capable of contributing to the germ line of the mammal of interest, and a targeting construct containing target-gene sequences with the desired mutation. ES cell lines are derived from the inner cell mass of a blastocyst-stage embryo. The targeting construct is transfected into cultured ES cells. Homologous recombination occurs in a number of the transfected cells, resulting in introduction of the mutation present in the targeting construct into the target gene. Once identified, mutant ES cell clones are microinjected into a normal blastocyst to produce a chimeric mammal, e.g. a chimeric mouse. Because ES cell lines retain the ability to differentiate into every cell type present in the mouse, the chimera can have tissues, including the germ line, with contribution from both the normal blastocyst and the mutant ES cells. Breeding germ-line chimeras yields animals that are heterozygous for the mutation introduced into the ES cell, and that can be interbred to produce homozygous mutant mice.

### Production of a Knock-Out (Gene-Targeting) Construct

One of two configurations of constructs are generally used for homologous recombination, i.e., an insertion constructs or a replacement construct. An insertion construct contains a region of homology to the target gene cloned as a single continuous sequence, and is linearized by cleavage of a unique restriction site within the region of homology. Homologous recombination introduces the insertion construct sequences into the homologous site of the target gene, interrupting normal target-gene structure by adding an additional sequence, for example, a LoxP site. Such a vector is useful for, for example, introducing on or more LoxP sites flanking a region of interest in the dynorphin gene or for introducing a mutation that alters, for example, the amino acid composition of dynorphin (e.g., for removing a cleavage site for a proteolytic enzyme).

A replacement construct is a more commonly used construct. Such a construct contains two regions of homology to the target gene located on either side of a heterologous nucleic acid (for example, encoding one or more positive selectable markers, such as, for example, a fluorescent protein (e.g. enhanced green fluorescent protein), β-galactosidase, an antibiotic resistance protein (e.g. neomycin resistance or zeocin resistance) or a fusion protein (e.g. the; β-galactosidase-neomycin resistance protein, β-geo,). Homologous recombination proceeds by a double cross-over event that replaces the target-gene sequences with the replacement-construct sequences (i.e. a region of the gene that occurs between the regions of homology with regions of the targeting construct are replaced with the heterologous nucleic acid).

The present invention provides a vector construct (e.g., a dynorphin targeting vector or dynorphin targeting construct) designed to disrupt the function of a wild-type (endogenous) mammalian dynorphin gene. In general terms, an effective dynorphin targeting vector comprises a nucleic acid comprising a nucleotide sequence that is effective for homologous recombination with the dynorphin gene. For example, a replacement targeting vector comprises a nucleic acid encodes a selectable marker gene flanked by regions of nucleic acid homologous to or substantially identical to a genomic sequence of dynorphin or a region thereof. For example, the selectable marker is flanked by a region homologous to or substantially identical to a region of the dynorphin genomic DNA 5′ to exon 3 and another region homologous to or substantially identical to a region of the dynorphin genomic DNA 3′ to exon 5.

One of skill in the art will recognize that any dynorphin genomic nucleotide sequence of appropriate length and composition to facilitate homologous recombination at a specific site that has been preselected for disruption can be employed to construct a dynorphin targeting vector. Guidelines for the selection and use of sequences are described for example in Deng and Cappecchi, Mol. Cell. Biol., 12:3365-3371, 1992 and Bollag, et al., Ann. Rev. Genet., 23:199-225, 1989. For example, a wild-type dynorphin gene is mutated and/or disrupted by inserting a recombinant nucleic acid sequence (e.g., a dynorphin targeting construct or vector) into all or a portion of the dynorphin gene locus. For example, a targeting construct is designed to recombine with a particular portion within the enhancer, promoter, coding region, start codon, non-coding sequence, introns or exons of the dynorphin gene. Alternatively, a targeting construct comprises a recombinant nucleic acid that introduces a stop codon after or within exon 1, 2, 3 and/or 4 of the dynorphin gene.

Suitable targeting constructs of the invention are prepared using standard molecular biology techniques known to those of skill in the art. For example, techniques useful for the preparation of suitable vectors are described by Maniatis, et al., Molecular Cloning: A Laboratory Manual, Cold Spring Harbor Laboratory, Cold Spring Harbor, N.Y. An appropriate vector includes, for example, an insertion vector such as the insertion vector described by Capecchi, M. R., Science, 244:1288-92, 1989; or a vector based on a promoter trap strategy or a polyadenylation trap, or “tag-and-exchange” strategy described by Bradley, et al., Biotechnology, 10:543-539, 1992; or Askew, et al., Mol. Cell. Biol., 13:4115-5124, 199.

One of skill in the art will readily recognize that any of a number of appropriate vectors known in the art can be used as the basis of a suitable targeting vector. In practice, any vector that is capable of accommodating the recombinant nucleic acid required for homologous recombination and to disrupt the target gene is useful. For example, pBR322, pACY164, pKK223-3, pUC8, pKG, pUC19, pLG339, pR290, pKC101 or other plasmid vectors can be used. Alternatively, a viral vector such as the lambda gt11 vector system is useful as the backbone (e.g. cassette) for a targeting construct.

In an example of the invention, the targeting vector comprises one or more recombination sites, such as, for example, a LoxP site (which is a recognition site of the P1 recombination enzyme Cre) or a frt site (which is a recognition site of the yeast recombinase flp). Methods for using such recombinase sites for the production of a targeting vector and for the production of a knockout mammal are known in the art and described, for example, in Fiering et al., 1995; Vooijs et al., 1998.

For example, If there are two loxP sites in the same orientation near each other in a nucleic acid, Cre removes the sequence between the two sites, leaving a single loxP site in the original DNA and a second loxP in a circular piece of DNA containing the intervening sequence. Accordingly, loxP sites or frt sites that are inserted flanking a region of the dynorphin gene or the entire dynorphin gene are useful for the removal of the intervening sequence.

Such a system is additionally useful for, for example, producing a conditional knockout animal. A conditionally silencing or knocking out a gene means that that the silencing of the gene is dependent upon an external stimulus that may be spatially and/or temporally controlled. For example, the dynorphin gene is flanked by two or more loxP sites (i.e., it is floxed) and upon expression of Cre recombinase the gene is removed. The expression of Cre may be spatially controlled (e.g., under the control of a tissue or cell specific promoter) and/or may be temporally controlled (e.g. under control of a promoter that is expressed at a certain stage of development or that is inducible, e.g., a tet inducible or repressible promoter).

For example, a mouse comprising a floxed dynorphin gene is crossed with a mouse expressing Cre operably connected with the uncoupling protein-1 promoter, that drives expression of Cre in brown adipose tissue. Accordingly, the expression of dynorphin is only silenced or reduced brown adipose tissue or cells that express a protein under control of the uncoupling protein-1 promoter.

As exemplified herein, recombination sites (e.g., loxP or frt) are also useful for removing a selectable marker following integration into the genome of a cell or organism. For example, a replacement targeting construct comprises one or more selectable markers flanked by recombination sites. Following successful targeting Cre is expressed (e.g. an expression construct that expresses Cre is introduced into a cell) or Cre is introduced into a cell or a mammal comprising the targeting construct is crossed with another mammal expressing Cre) and the selectable marker is removed. Such a system is useful for, for example, removing a region of a targeting construct that includes, for example, a promoter that may alter the expression of genes other than the gene of interest or encodes a polypeptide that is toxic to a subject or unwanted.

The present inventors have provided a knockout construct that comprises a plurality of selectable markers, e.g., enhanced green fluorescent protein, neomycin resistance and zeocin resistance, in addition to Cre under control of the tetracycline responsive element (TRE). The construct additionally comprises a retroviral tetracycline repression element (rtetR) that was under control of dynorphin expression following homologous recombination. The construct additionally comprises loxP sites arranged to excise the enhanced green fluorescent protein gene, the neomycin resistance gene the zeocin resistance gene, and the Cre gene upon Cre expression. Expression of the rtetR element in the presence of tetracycline or doxycycline induces expression of Cre thereby removing the selectable markers from the genome of the cell or organism. The cassette comprising each of the previously discussed features comprises the sequence set forth in SEQ ID NO: 6.

In one example, a construct of the present invention is used to replace all or part of the nucleic acid of a non-human mammalian gene which encodes the dynorphin polypeptide. For example, the targeting construct is used to replace exons 1, 2, 3 and 4 of the dynorphin gene. In another example, exons 3 and 4 of the dynorphin gene are disrupted. Exons 3 and 4 encode pre-pro-dynorphin which is post-translationally modified to produce dynorphin A-(1-17), leu-enkephalin (DYN-(1-5)), DYN-(1-8), DYN-(1-13), dynorphin-B and α-neo-endorphin in addition to all other peptides or polypeptides produced from pre-pro-dynorphin.

In an example, the exemplified knock-out construct is modified or another knock-out construct produced using methods known in the art and/or described herein to reduce, suppress or ablate the expression of one or more specific forms of dynorphin in a mammal. For example, a knockout construct is produced to produce a genetically modified animal selected from the group consisting of dynorphin A/^{-/-}, leu-enkephalin^{-/-}, DYN-(1-8)^{-/-}, DYN-(1-13)^{-/-}, dynorphin-B^{-/-}, α-neo-endorphin^{-/-} and combinations thereof.

Alternatively, an insertional gene targeting vector is used to introduce a mutation that reduces or suppresses the post translational modification of a dynorphin protein or a modified form thereof. For example, a mutation is introduced into the sequence of dynorphin to alter the amino acid sequence of a cleavage site of a preoteolytic enzyme. For example, aminopeptidase cleaves dynorphin A between Arg6 and Arg7 or between Gly3 and Phe4. Accordingly, mutation of the nucleic acid encoding any of these sites that results in altering any one or more of these amino acids reduces or suppresses cleavage by the aminopeptidase. The present invention clearly contemplates insertional vectors useful for production of such a genetically modified animal.

### Production of a Dynorphin^{-/-}Cell

Following production of a suitable gene construct, said construct is introduced into the relevant cell. Methods of introducing the gene constructs into a cell for expression are well known to those skilled in the art and are described for example, in Ausubel et al (In: Current Protocols in Molecular Biology. Wiley Interscience, ISBN 047 150338, 1987) and (Sambrook et al (In: Molecular Cloning: Molecular Cloning: A Laboratory Manual, Cold Spring Harbor Laboratories, New York, Third Edition 2001). Means for introducing recombinant DNA into cells include, but are not limited to electroporation, microinjection, transfection mediated by DEAE-dextran, transfection mediated by calcium phosphate, transfection mediated by liposomes such as by using Lipofectamine (Invitrogen) and/or cellfectin (Invitrogen), transduction by Adenoviuses, Herpesviruses, Togaviruses or Retroviruses and microparticle bombardment such as by using DNA-coated tungsten or gold particles (Agacetus Inc., WI, USA). For example, a cell is electroporated with a targeted construct of the invention.

A suitable cell for the production of a knockout animal is, for example, an embryonic stem cell. An embryonic stem cell is a pluripotent cell isolated from the inner cell mass of mammalian blastocyst. ES cells can be cultured in vitro under appropriate culture conditions in an undifferentiated state and retain the ability to resume normal in vivo development as a result of being combined with blastocyst and introduced into the uterus of a pseudopregnant foster mother. Those of skill in the art will recognize that various stem cells and stem cell lines are known in the art, such as, for example, AB-1, HM-1, D3. CC1.2, E-14T62a, RW4 or JI (Teratomacarcinoma and Embryonic Stem Cells: A Practical Approach, E. J. Roberston, ed., IRL Press). Clearly, a suitable stem cell or stem cell line will depend upon the type of knockout mammal to be produced. For example, should a knockout mouse be desired a mouse ES cell line is used. Furthermore, should an inbred strain of knockout mice be preferred, an ES cell line derived from the same strain of mice that is to be used is preferred.

Following transfection cells are maintained under conditions sufficient for homologous recombination to occur while maintaining the pluripotency of the ES cell.

In an example of the invention, an ES cell is selected that has homologously recombined to introduce the targeting vector into it's genome (as opposed to random integration). A method used for eliminating cells in which the construct integrated into the genome randomly, thus further enriching for homologous recombinants, is known as positive-negative selection. Such methods are described, for example, in U.S. Pat. No. 5,464,764. Briefly, constructs useful for positive-negative selection comprise a negative selectable marker (e.g., herpes simplex virus thymidine kinase, HSV-TK) outside the region of homology to the target gene (i.e. in a region that will not be incorporated into the genome of a cell following homologous recombination). In the presence of the TK gene, the cells are sensitive to acyclovir and its analogs (e.g., gancyclovir, GANC). The HSV-TK enzyme activates these drugs, resulting in their incorporation into growing DNA, causing chain termination and cell death. During homologous recombination, sequences outside the regions of homology to the target gene are lost due to crossing over. In contrast, during random integration substantially all of the sequences in the construct are retained as recombination usually occurs at the ends of the construct. The presence of the TK gene can be selected against by growing the cells in gancyclovir; the homologous recombinants will be G418-resistant and gancyclovir-resistant, whereas clones in which the construct integrated randomly will be G418-resistant and gancyclovir-sensitive.. Other markers that are lethal to cells have also been used instead of TK and gancyclovir (e.g., diphtheria toxin; Yagi et al., 1990).

Alternatively, or in addition, a cell is screened using, for example, PCR or Southern blotting to determine a targeting construct that has integrated into the correct region of the genome rather than randomly integrated. Methods for such screening are known in the art, and described, for example, in Nagy et al eds. Manipulating the Mouse Embryo, Cold Spring Harbor Laboratory, 3rd Edition, 2002, ISBN 0879695749 and Tymms, Kola eds Gene Knockout Protocols, Humana Press, 2001, ISBN: 0896035727, Ausubel et al (In: Current Protocols in Molecular Biology. Wiley Interscience, ISBN 047 150338, 1987) and (Sambrook et al (In: Molecular Cloning: Molecular Cloning: A Laboratory Manual, Cold Spring Harbor Laboratories, New York, Third Edition 2001).

Following selection of a cell that has integrated the targeting vector, should the vector comprise recombination sites, Cre may be introduced or expressed in the cells to remove regions of the genome, should this be desired.

Production of a Dynorphin−/− mouse

Following production of an ES cell in which at least one copy of the dynorphin gene has incorporated the targeting construct the cell is preferably grown to form an ES cell colony using methods known in the art. One or more cells from the colony are then used to produce a chimeric animal.

An example of a method used to generate chimeras involves the injection of the genetically modified ES cells into the blastocoel cavity of a developing embryo. For example, should the targeted ES cell be of mouse origin, an ES cell is injected into the blastocoel cavity of a 3.5-day-old mouse embryo. The injected embryo is surgically implanted into the uterus of a foster mother, for example, a pseudopregant female. A resultant offspring is a chimera as its tissues is derived from both the host embryo and from the ES cell. Should the ES cell populate the germ line, the chimera can pass an altered gene to offspring, resulting in a new mouse strain in which all cells contain an altered gene.

By breeding a mouse of the new mouse strain with a wild-type mouse offspring that are heterozygous for the mutation are produced, i.e., dynorphin^{+/+}. However breeding two heterozygous mice, or two homozygous mice or a heterozygous mouse and a homozygous mouse produces at least some offspring that are homozygous for the mutation, i.e., dynorphin^{-/-}.

The present invention clearly contemplates both heterozygous and homozygous knockout non-human mammals. For example, the present invention contemplates a genetically modified non-human mammal with a phenotype selected from the group consisting of dynorphin^{-/+}, dynorphin^{-/-}, dynorphin^{+/loxP}, dynorphin^{loxP/laxP}, dynorphin^{+/frt} and dynorphin^{frt/frt}. As used herein, the term “−/−-” shall be understood to symbolize a mouse that is homologous for a mutation that silences a gene; “−/+” shall be taken to symbolize a mouse that is heterozygous for a mutation that silences a gene; “+/loxP” shall be taken to symbolize a mouse that is heterozygous for a gene or region thereof or that comprises a targeting construct or region thereof that is flanked by loxP sites; “lox/lox” shall be taken to symbolize a mouse that is homozygous for a gene or region thereof or that comprises a targeting construct or region thereof that is flanked by loxP sites; “+/frt” shall be taken to symbolize a mouse that is heterozygous for a gene or region thereof or that comprises a targeting construct or region thereof that is flanked by frt sites; and “frt/frt” shall be taken to symbolize a mouse that is homozygous for a gene or region thereof or that comprises a targeting construct or region thereof that is flanked by frt sites.

It is to be understood that the dynorphin knockout mammals described herein can be produced by methods other than the embryonic stem cell method described above, for example by the pronuclear injection of recombinant genes into the pronuclei of one-cell embryos or other gene targeting methods which do not rely on the use of a transfected ES cell, and that the exemplification of the single method outlined above is not intended to limit the scope of the invention to animals produced solely by this protocol.

As will be apparent from the preceding discussion, the present invention contemplates a non-human mammal (e.g. a mouse) that has been genetically modified to reduce the expression of any one or more forms of dynorphin, or a derivative thereof. For example, a modified form of the targeting vector described herein is useful for, for example, producing a mouse with reduced expression of dynorphin or a derivative thereof selected from the group consisting of pre-pro-dynorphin, pro-dynorphin, dynorphin, dynorphin A-(1-17), leu-enkephalin (DYN-(15)), DYN-(1-8), DYN-(1-13), dynorphin-B, α-neo-endorphin, β-neo-endorphin, a derivative of pre-pro-dynorphin, a derivative of pro-dynorphin, a derivative of dynorphin, a derivative of dynorphin A-(1-17), a derivative of leu-enkephalin (DYN-(1-5)), a derivative of DYN-(1-8), a derivative of DYN-(1-13), a derivative of dynorphin-B, a derivative of α-neo-endorphin, a derivative of β-neo-endorphin, a region of pre-pro-dynorphin, a region of pro-dynorphin, a region of dynorphin, a region of dynorphin A-(1-17), a region of leu-enkephalin (DYN-(1-5)), a region of DYN-(1-8), a region of DYN-(1-13), a region of dynorphin-B, a region of α-neo-endorphin, a region of β-neo-endorphin and mixtures thereof. The present inventors have clearly demonstrated production of a mouse in which the expression of all forms of dynorphin or derivatives thereof are reduced. For example, the present inventors have demonstrated the production of a mouse in which expression of all forms of dynorphin encoded by a nucleic acid comprising the sequence set forth in SEQ ID NO: 3 is reduced.

The present invention additionally contemplates a cell, a cell line, a cell culture, a primary tissue, a cellular extract or a cell organelle isolated from a dynorphin knockout mammal of the instant invention. For example, a cell culture, or cell line or cell is derived from any desired tissue or cell-type from a dynorphin knockout mouse. For example, a cell culture, or cell line or cell is derived a tissue or cell-type that express high levels of dynorphin in nature. For example, a dynorphin knockout mammal produced in accordance with the present invention is utilized as a source of cells for the establishment of cultures or cell lines (e.g., primary, immortalized) useful for determining a dynorphin mimetic compound.

In Vitro Assays for Determining Compounds that Bind to Opioid Receptors or Agonize or Antagonize the Action of One or More Dynorphin Peptides

A number of art-recognized assays are available that are suitable for the high-throughput determination of compounds that modulate binding of dynorphin to an opioid receptor and/or agonize the activation of an opioid receptor by a dynorphin peptide and/or antagonize the activation of an opioid receptor by a dynorphin peptide. Such assays are particularly useful e.g., as primary screens to identify a panel of compounds for testing in the animal model of the present invention and/or for validation of compounds identified in in vivo screens using the dynorphin knockout animal of the invention. Examples of such in vitro and in situ assays are described in the following paragraphs.

### 1. Ligand Binding Assay

Ligand binding assays for determining dynorphin binding to opioid receptors are well-known in the art and described for example by Zhang et al., J. Pharmacol. Exp Ther. 286, 136-141, 1998, and Tang et al., J. Neurophysiol. 83, 2610-2612, 2000. In a modification of such an art-recognized assay, the ability of a compound being screened to antagonize or agonize the binding of a dynorphin peptide to one or more opioid receptors is determined, using either whole cells expressing the receptor or membrane fractions derived from the cells.

The cells can be cells that express an endogenous opioid receptor, or more conveniently, cells stably or transiently transfected with nucleic acid encoding one or more opioid receptors such that they express a functional opioid receptor in their membrane. For transfection, mammalian cells, e.g., H9.10 cells, AV12 cells, HEK293 cells, etc, or Xenopus oocytes, are preferred. The cells are transfected with receptor cDNAs cloned in a suitable expression vector e.g., pcDNA3 (Stratagene, La Jolla, Calif.). Any standard transfection method may be used, such as the calcium phosphate method (Chen and Okayama, Mol. Cell Biol. 7, 2745-2752, 1987). Cells stably or transiently expressing the receptors are isolated.

For receptor binding, a dynorphin peptide such as, for example, dynorphin A (Peninsula Laboratories Inc., Belmont, Calif.), is preferably labelled with a suitable reporter molecule e.g. a radioligand such as [³H] or [¹⁴C] or [³⁵S] or biotin. The peptide is then contacted with whole cells or membrane fractions of cells under conditions sufficient for the dynorphin peptide to bind to an opioid receptor in the presence and absence of the compound being tested and the reporter molecule is detected.

For example, labelled dynorphin A may be used as a labelled ligand wherein binding of said ligand to membrane proteins (about 10-75 μg/reaction) is performed at about 4° C. for 2.5 hr in a suitable binding buffer (e.g., 50 mM Tris/HCl, pH7.4 and 0.5% bovine serum albumin, 1 mM PMSF, 10 μg/ml leupeptin, 100 μg/ml benzamidine, 100 μg/ml trypsin inhibitor). After a suitable reaction time, reactions are terminated, such as by vacuum filtration through Whatman GF/B filters pre-treated with 0.2% polyethylenimine, the filters are washed, and the amount of bound reporter molecule is determined. Non-specific binding can be determined in such assays as the amount of labelled dynorphin peptide that binds to the receptors in the presence of a high concentration of naxolone, which is a competitive inhibitor of dynorphin binding to opiate receptors.

2. Assays that Measure Dynorphin Action

Assays for determining the activity of dynorphin peptides are well-known in the art and described for example by Zhang et al., J. Pharmacol. Exp Ther. 286, 136-141, 1998, and Tang et al., J. NeurophysioL 83, 2610-2612, 2000. In a modification of such an art-recognized assay, the ability of a compound being screened to modify dynorphin action is determined, using either whole cells expressing the receptor or membrane fractions derived from the cells or isolated neuronal cells or cultured neurons.

In this context, the cells can be any cells that mediate the action of dynorphin in such a manner as to produce a measurable read-out. For example, wherein the readout is mediated by an opioid receptor, cells that express an endogenous opioid receptor, or more conveniently, cells stably or transiently transfected with nucleic acid encoding one or more opioid receptors such that they express a functional opioid receptor in their membrane are preferred. Alternatively, wherein the assay readout may not be mediated by opioid receptor(s), it is preferred to use a primary neuronal culture that has been demonstrated to produce a dynorphin-mediated effect.

For transfection, mammalian cells, e.g., H9.10 cells, AV12 cells, HEK293 cells, etc, or Xenopus oocytes, are preferred. The cells are transfected with receptor cDNAs cloned in a suitable expression vector e.g., pcDNA3 (Stratagene, La Jolla, Calif.). Any standard transfection method may be used, such as the calcium phosphate method (Chen and Okayama, Mol. Cell Biol, 7, 2745-2752, 1987). Cells stably or transiently expressing the receptors are then isolated.

Preferred sources of primary neuronal cultures include hippocampal CA3 region e.g., mossy fibers, brain cortex, etc. The tissue is excised from an animal, minced or ground and dispersed to release cells and cell aggregates, which are then transferred to culture vessels and maintained in a suitable medium e.g., minimal essential medium (MEM) supplemented with nutrients (e.g., 0.4% glucose, 10% fetal calf serum, 10% horse serum, lOmM KCI). Primary cultures are maintained for the duration of the screening procedure. Preferably, they are prepared fresh for each assay batch.

Preferred assay readouts from transfect cells or primary neuronal cultures include electrophysiological determinations e.g., voltage clamping to measure K+ current, or measurement of intracellular calcium concentration.

### a) Electrophysiological Determination of Opioid Receptor Activation

Without limiting the present invention, cells expressing one or more opioid receptors are voltage-clamped at about −80 mV with two glass electrodes filled with 3M KCl and having a resistance of about 2 to 3 MΩ, e.g., using an Axoclamp-2A (Axon Instruments) under the control of pCLAMP software (Axon Instruments). Cells are then superfused with either ND96 (96 mM NaCl, 2 mM KCl, 1 mM MgCl₂, 1.5 mM CaCl₂ and 5 mM HEPES, pH 7.5) or a high potassium solution (96 mM KCl, 2 mM NaCl, 1 mM MgCl₂, 1.5 mM CaCl₂ and 5 mM HEPES, pH 7.5) in the presence and absence of an amount of a compound, or an amount of a pool of compounds, to be tested. Membrane currents are recorded for both samples (i.e., in the presence and absence of the compound(s)), for example, with the aid of the pCLAMP software using a Gould chart recorder. Data can also be analyzed using pCLAMP software. A difference in the inwardly rectifying K+current in the presence and absence of the compound(s) being tested indicates the presence of a compound that modulates dynorphin action.

### b) Measurement of Intracellular Calcium

For determining changes in intracellular calcium, cells such as but not limited to cultured primary neuronal cells are loaded with a detectable label, such as the fluorescent label Fluo-3/AM, in medium (e.g., MEM) and incubated in a suitable calcium-comprising buffer (e.g., 10 mM HEPES, 25 mM glucose, 137 mM NaCl, 10 mM KCl, and 3 mM CaCl₂, pH 7.4) in the presence and absence of a compound or pool of compounds to be tested. Intracellular calcium concentration is then determined, wherein a difference in calcium concentration in the presence and absence of the compound(s) being tested indicates that the compound modulates dynorphin action.

For example, in the case of fluorescent labels, fluorescence images are acquired by standard photometrics e.g., using CCD camera and standard optics (Optical, Brattleboro, Vt.) for Fluo-3, including a 10-nm band-pass excitation filter centred at 480 nm and a 15-nm band-pass emission filter centred at 530 nm. Digitized output of the CCD camera is analyzed using customized software e.g., Silicon Graphics IRIS 10/900. Fluorescence intensity within a single cell is quantified, such as by acquiring sequential images with a constant exposure time (e.g., about 300 msec) sufficient to provide images with fluorescence intensities above cell background and without causing saturation of the imaging elements. For each cell, images may be taken before, i.e., resting Ca²⁺, and after administration of compound(s) (sample fluorescence). The maximal Fluo-3 fluorescence response for each cell is generally determined by depolarizing the cell with the addition of 40 mM KCl at the end of the experiment. The fluorescence intensity within a cell may be expressed as the mean IOD averaged over the area of the cell body. Changes in intracellular calcium concentration are expressed as a percentage of the maximal fluorescence response induced by 40 mM KCl by the following equation:

(sample−basal)/(maximal−basal)×100%.

This approach allows for comparison of each cell response independent of differences in dye concentration. Statistical significance is determined e.g., at the 95% confidence level, by unpaired t-test or by two-way analysis of variance (ANOVA).

### Compounds That Modulate a Phenotype of the Dynorphin-Deficient Animal Model

The range of compounds contemplated herein for modulating a phenotype of the dynorphin-deficient animal model described herein includes peptides derived from preprodynorphin and capable of complementing the preprodynorphin-deficiency; non-dynorphin peptides, such as, for example dynorphin peptidomimetics; small organic molecules, such as, for example derived from publicly available combinatorial libraries; and nucleic acids, including nucleic acid encoding said peptide derived from preprodynorphin or said non-preprodynorphin peptide.

In one embodiment, the compounds are formulated for administration to a non-human animal or a human. The formulations can be suitable for administration by injection by a subcutaneous, intravenous, intranasal, or intraperitoneal route. Alternatively, they can be suitable for oral administration in the form of feed additives, tablets, troches, etc.

The compounds are conveniently formulated in a suitable excipient or diluent, such as, for example, an aqueous solvent, non-aqueous solvent, non-toxic excipient, such as a salt, preservative, buffer and the like. Examples of non-aqueous solvents are propylene glycol, polyethylene glycol, vegetable oil and injectable organic esters such as ethyl oleate. Aqueous solvents include water, alcoholic/aqueous solutions, saline solutions, parenteral vehicles such as sodium chloride, Ringer's dextrose, etc. Preservatives include antimicrobial, anti-oxidants, chelating agents and inert gases. The pH and exact concentration of the various components the formulation suitable for administration to the animal are adjusted according to routine skills in the art. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavouring agent such as peppermint, methyl salicylate, or orange flavouring. For administration by inhalation, the compounds are delivered in the form of an aerosol spray from pressured container or dispenser which contains a suitable propellant, e.g., a gas such as carbon dioxide, or a nebulizer. For trans-mucosal or transdermal administration, penetrants appropriate to the barrier to be permeated are used in the formulation. Such penetrants are generally known in the art, and include, for example, for trans-mucosal administration, detergents, bile salts, and fusidic acid derivatives. Trans-mucosal administration can be accomplished through the use of nasal sprays or suppositories. For transdermal administration, the active compounds are formulated into ointments, salves, gels, or creams as generally known in the art.

Optionally, the formulation will also include a carrier, such as, for example, bovine serum albumin (BSA), keyhole limpet hemocyanin (KLH), ovalbumin, mouse serum albumin, rabbit serum albumin and the like. Means for conjugating peptides to carrier proteins are also well known in the art and include glutaraldehyde, m-maleimidobencoyl-N-hydroxysuccinimide ester, carbodiimide and bis-biazotized benzidine.

In another embodiment, the subject method further comprises producing or synthesizing the compound that is tested on the genetically modified animal.

Peptidyl compounds are conveniently made by standard peptide synthesis, such as the Merrifield method of synthesis (Merrifield, J Am Chem Soc, 85,:2149-2154, 1963) and the myriad of available improvements on that technology (see e.g., Synthetic Peptides: A User's Guide, Grant, ed. (1992) W.H. Freeman & Co., New York, pp. 382; Jones (1994) The Chemical Synthesis of Peptides, Clarendon Press, Oxford, pp. 230.); Barany, G. and Merrifield, R. B. (1979) in The Peptides (Gross, E. and Meienhofer, J. eds.), vol. 2, pp. 1-284, Academic Press, New York; Wünsch, E., ed. (1974) Synthese von Peptiden in Houben-Weyls Metoden der Organischen Chemie (Müler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474.

Preferably, the peptide is synthesized on a solid phase support, such as, for example, a polystyrene gel bead comprising polystyrene cross-linked with divinyl benzene, preferably 1% (w/w) divinyl benzene, which is further swollen using lipophilic solvent, such as, for example dichloromethane or dimethylformamide (DMF). The polystyrene can be functionalized by addition of chloromethane or amino methyl groups. Alternatively, cross-linked and functionalized polydimethyl-acrylamide gel can be used once swollen and solvated using DMF or dipolar aprotic solvent. Other solid phase supports known to those skilled in the art can also be used for peptide synthesis, such as, for example, polyethylene glycol-derived bead produced by grafting polyethylene glycol to the surface of inert polystyrene beads. Preferred commercially available solid phase supports include PAL-PEG-PS, PAC-PEG-PS, KA, KR, or TGR (Applied Biosystems, CA 94404, USA).

For solid phase peptide synthesis, blocking groups that are stable to the repeated treatments necessary for removal of the amino blocking group of the growing peptide chain and for repeated amino acid couplings, are used for protecting the amino acid side-chains during synthesis and for masking undesired reactivity of the α-amino, carboxyl or side chain functional groups. Blocking groups (also called protecting groups or masking groups) thus protect the amino group of the amino acid having an activated carboxyl group that is involved in the coupling reaction, or protect the carboxyl group of the amino acid having an acylated amino group that is involved in the coupling reaction.

During synthesis, coupling occurs following removal of a blocking group without the disruption of a peptide bond, or any protecting group attached to another part of the peptide. Additionally, the peptide-resin anchorage that protects the C-terminus of the peptide is protected throughout the synthetic process until cleavage from the resin is required. Accordingly, by the judicious selection of orthogonally protected α-amino acids, amino acids are added at desired locations to a growing peptide whilst it is still attached to the resin.

Preferred amino blocking groups are easily removable but sufficiently stable to survive conditions for the coupling reaction and other manipulations, such as, for example, modifications to the side-chain groups. In one embodiment, amino blocking groups are selected from the group consisting of: (i) a benzyloxycarbonyl group (Z or carbocenzoxy) that is removed easily by catalytic hydrogenation at room temperature and ordinary pressure, or using sodium in liquid ammonia and hydrobromic acid in acetic acid; (ii) a urethane derivative; (iii) a t-Butoxycarbonyl group (Boc) that is introduced using t-butoxycarbonyl azide or di-tert-butyldicarbonate and removed using mild acid such as, for example, trifluoroacetic acid (50% TFA in dichloromethane), or HCl in acetic acid/dioxane/ethylacetate; (iv) a 9-fluorenylmethyloxycarbonyl group (Fmoc) that is cleaved under mildly basic, non-hydrolytic conditions, such as, for example, using a primary or secondary amine (eg. 20% piperidine in dimethyl formamide); (v) a 2-(4-biphenylyl) propyl(2)oxycarbonyl group (Bpoc); (vi) a 2-nitro-phenylsulfenyl group (Nps); and (vii) a dithia-succionyl group (Dts). Boc is widely used to protect the N-terminus in Fmoc chemistry, or Fmoc is widely used to protect the N-terminus in Boc chemistry.

Side chain-protecting groups will vary for the functional side chains of the amino acids forming the peptide being synthesized. Side-chain protecting groups are generally based on the Bzl group or the tBu group. Amino acids having alcohols or carboxylic acids in the side-chain are protected as Bzl ethers, Bzl esters, cHex esters, tBu ethers, or tBu esters. Side-chain protection of Fmoc amino acids requires blocking groups that are ideally base stable and weak acid (TFA) labile. Many different protecting groups for peptide synthesis have been described (see The Peptides, Gross et aL. eds., Vol. 3, Academic Press, New York, 1981). For example, the 4-methoxy-2,3,6-trimethylphenylsulfonyl (Nd-Mtr) group is useful for Arginine side-chain protection, however deprotection of Arg(Mtr) requires prolonged TFA treatment. A number of soft acid (TFA, thalium (III) trifluoroacetate/TFA) labile groups, or TFA stable but thalium (III) trifluoroacetate/TFA labile groups, or soft acid stable groups are used to protect Cystine.

The two most widely used protection strategies are the Boc/Bzl- and the Fmoc/tBu-strategies. In Boc/Bzl, Boc is used for amino protection and the side-chains of the various amino acids are protected using Bzl- or cHex-based protecting groups. A Boc group is stable under catalytic hydrogenation conditions and is used orthogonally along with a Z group for protection of many side chain groups. In Fmoc/tBu, Fmoc is used for amino protection and the side-chains are protected with tBu-based protecting groups.

Alternatively, the peptidyl compound is produced by the recombinant expression of nucleic acid encoding the amino acid sequence of said peptide. Random peptide-encoding libraries are particularly preferred for such purposes, because they provide a wide range of different compounds to test. Alternatively, naturally-occurring nucleic acids can be screened. According to this embodiment, nucleic acid encoding the peptidyl compound is produced by standard oligonucleotide synthesis or derived from a natural source and cloned into a suitable expression vector in operable connection with a promoter or other regulatory sequence capable of regulating expression in a cell-free system or cellular system..

Oligonucleotides are preferably synthesized with linker or adaptor sequences at the 5′- and 3′-ends to facilitate subsequent cloning into a suitable vector system using standard techniques.

Placing a nucleic acid molecule under the regulatory control of, i.e., “in operable connection with”, a promoter sequence means positioning said molecule such that expression is controlled by the promoter sequence, generally by positioning the promoter 5′ (upstream) of the peptide-encoding sequence.

The prerequisite for producing intact peptides in bacteria such as E. coli is the use of a strong promoter with an effective ribosome binding site. Typical promoters suitable for expression in bacterial cells such as E. coli include, but are not limited to, the lacz promoter, temperature-sensitive λ_{L} or λ_{R} promoters, T7 promoter or the IPTG-inducible tac promoter. A number of other vector systems for expressing the nucleic acid molecule of the invention in E. coli are well-known in the art and are described, for example, in Ausubel et al (In: Current Protocols in Molecular Biology. Wiley Interscience, ISBN 047150338, 1987) or Sambrook et al (In: Molecular cloning, A laboratory manual, second edition, Cold Spring Harbor Laboratory, Cold Spring Harbor, N.Y., 1989). Numerous plasmids with suitable promoter sequences for expression in bacteria and efficient ribosome binding sites have been described, such as for example, pKC30 (λ_{L}: Shimatake and Rosenberg, Nature 292, 128, 1981); pKK173-3 (tac: Amann and Brosius, Gene 40, 183, 1985), pET-3 (T7: Studier and Moffat, J. Mol. Biol. 189, 113, 1986); the pBAD/TOPO or pBAD/Thio-TOPO series of vectors containing an arabinose-inducible promoter (Invitrogen, Carlsbad, Calif.), the latter of which is designed to also produce fusion proteins with thioredoxin to enhance solubility of the expressed protein; the pFLEX series of expression vectors (Pfizer Inc., CT, USA); or the pQE series of expression vectors (Qiagen, CA), amongst others.

Typical promoters suitable for expression in viruses of eukaryotic cells and eukaryotic cells include the SV40 late promoter, SV40 early promoter and cytomegalovirus (CMV) promoter, CMV IE (cytomegalovirus immediate early) promoter amongst others. Preferred vectors for expression in mammalian cells (eg. 293, COS, CHO, 10T cells, 293T cells) include, but are not limited to, the pcDNA vector suite supplied by Invitrogen, in particular pcDNA 3.1 myc-His-tag comprising the CMV promoter and encoding a C-terminal 6×His and MYC tag; and the retrovirus vector pSRαtkneo (Muller et al., Mol. Cell. Biol., 11, 1785, 1991). The vector pcDNA 3.1 myc-His (Invitrogen) is particularly preferred for expressing peptides in a secreted form in 293T cells, wherein the expressed peptide or protein can be purified free of conspecific proteins, using standard affinity techniques that employ a Nickel column to bind the protein via the His tag.

A wide range of additional host/vector systems suitable for expressing peptides are available publicly, and described, for example, in Sambrook et al (In: Molecular cloning, A laboratory manual, second edition, Cold Spring Harbor Laboratory, Cold Spring Harbor, N.Y., 1989).

Means for introducing the nucleic acid or a gene construct comprising same into a cell for expression are well-known to those skilled in the art. The technique used for a given organism depends on the known successful techniques. Means for introducing recombinant DNA into animal cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

Techniques for synthesizing small organic compounds will vary considerably depending upon the compound, however such methods will be well known to those skilled in the art. In one embodiment, informatics is used to select suitable chemical building blocks from known compounds, for producing a combinatorial library. For example, QSAR(Quantitative Structure Activity Relationship) modelling approach uses linear regressions or regression trees of compound structures to determine suitability. The software of the Chemical Computing Group, Inc.(Montreal, Canada) uses high-throughput screening experimental data on active as well as inactive compounds, to create a probabilistic QSAR model, which is subsequently used to select lead compounds. The Binary QSAR method is based upon three characteristic properties of compounds that form a “descriptor” of the likelihood that a particular compound will or will not perform a required function: partial charge, molar refractivity (bonding interactions), and logP (lipophilicity of molecule). Each atom has a surface area in the molecule and it has these three properties associated with it. All atoms of a compound having a partial charge in a certain range are determined and the surface areas (Van der Walls Surface Area descriptor) are summed. The binary QSAR models are then used to make activity models or ADMET models, which are used to build a combinatorial library. Accordingly, information from known appetite suppressants and non-suppressants, including lead compounds identified in initial screens, can be used to expand the list of compounds being screened to thereby identify highly active compounds.

The present invention is further described with reference to the following examples and the accompanying drawings.

### EXAMPLE 1

### Production of a Dynorphin Knockout Mouse

A 129/SvJ genomic bacterial artificial chromosome (BACS) library (Genome Systems, St. Louis, USA) was screened under high stringency conditions using a probe specific to the mouse Dyn (dynorphin) gene. Clones detected by the probe were isolated and mapped using restriction endonuclease digestion. For example, the BACs were mapped using EcoRI digestion and/or BstEll digestion or EcoRi digestion and/or NdeI digestion, amongst other restriction endonucleases.

Fragments produced by EcoRI digestion were subcloned into the pBluescript vector (Stratagene) and used as the 5′ arm of a targeting construct.

Nucleic acid encoding the retroviral tetracycline repressible element (rtetR) was cloned such that the start codon of dynorphin forms the start codon of rtetR. Accordingly, expression of this region produces the doxycycline sensitive repressor gene product.

Nucleic acid encoding the enhanced green fluorescent protein was then cloned to the rtetR fragment, such that the reading frame of eGFP is in the opposite orientation to rtetR. The start codon of eGFP was mutated by insertion of a 34bp Cre-recombinase recognition (loxP) site. Fragments containing a neomycin gene and a Zeocin resistance gene were then added to the 3′ end of the construct.

A fragment encoding Cre recombinase in which the stop codon was mutated by insertion of a loxP site in the same orientation as that inserted into eGFP was then inserted. Expression of the Cre gene was controlled by the tetracycline responsive element (TRE). The sequence of the cassette inserted between the two dynorphin arms is set forth in SEQ ID NO: 6 (i.e. the region from the start codon to the end of the tetracycline responsive element, TRE).

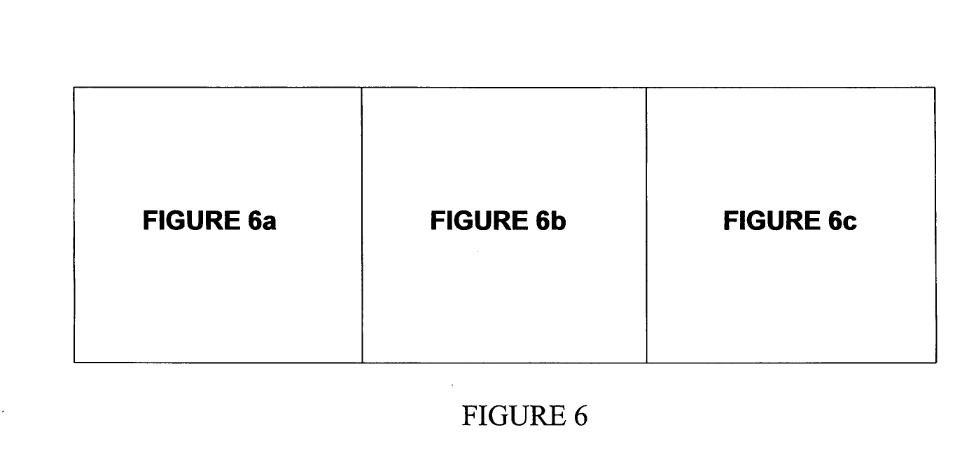

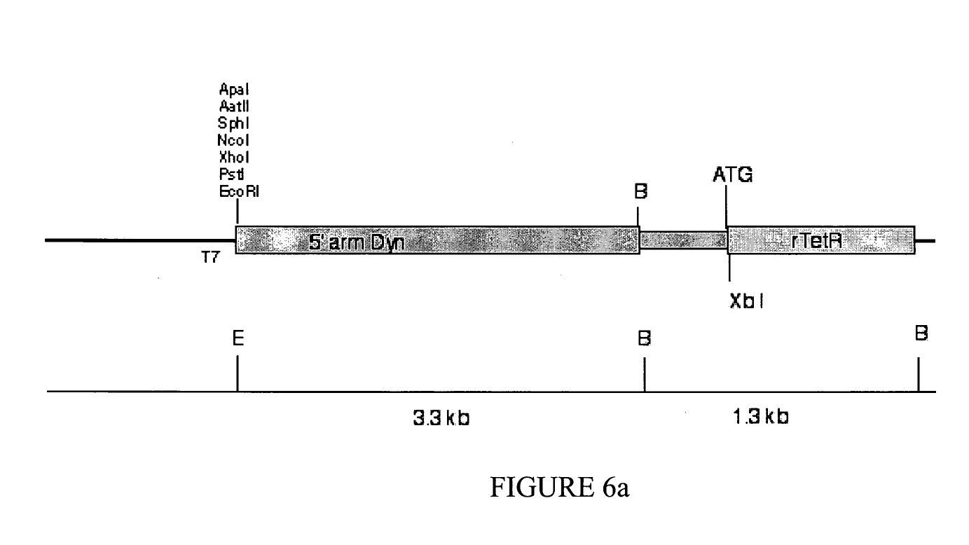

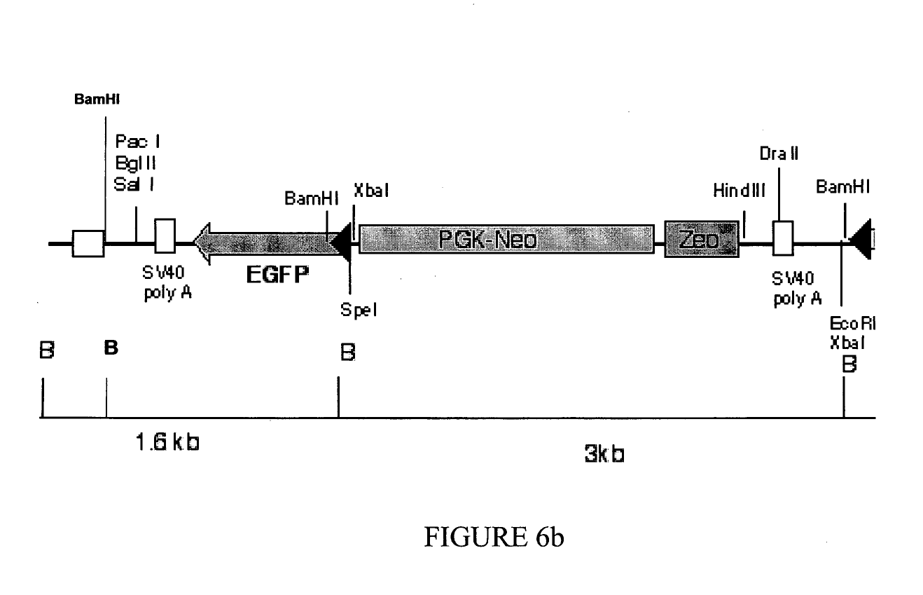

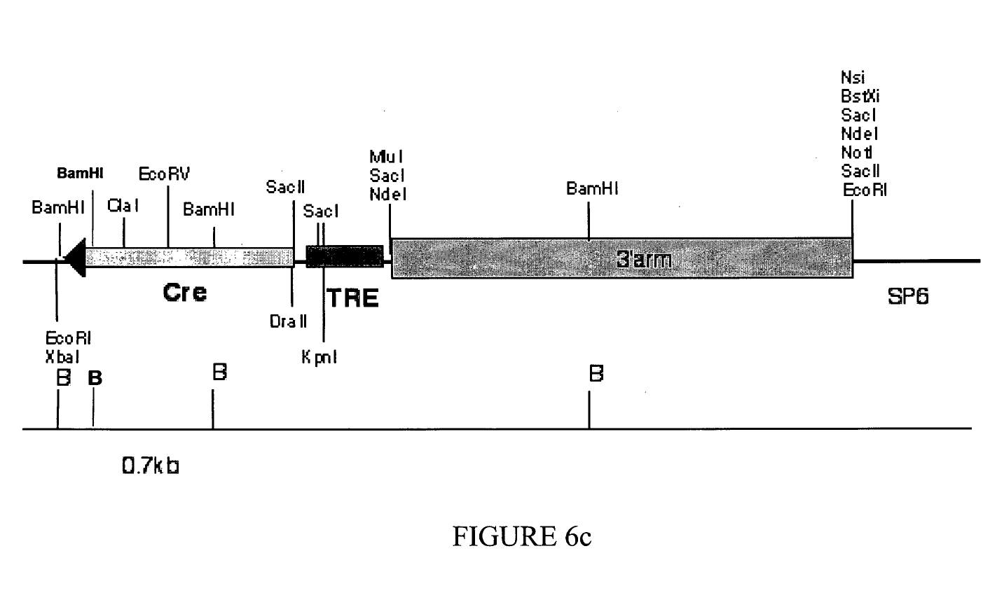

Following the Cre recombinase encoding fragment, a NdeI/EcoRI fragment of dynorphin was inserted. A diagrammatic representation of this targeting construct is depicted in FIG. 6. When this construct homologously recombines with the genome of a cell it removes the entire coding sequence of the pro-dynorphin gene consisting of exons 3 and 4.

The knockout construct was then transfected into mouse ES cells. ES cells were incubated in the presence of ES/LIF (leukemia inhibitory factor) medium and passaged every 2 to 3 days by seeding a 100-mm gelatin-coated tissue culture plate with 1-2×10⁶ cells/plate.

Cells were harvested and dissociated to single cells. Cells were then pelleted by centrifugation, washed and resuspended in a buffer suitable for electroporation. Cells are then electroporated with approximately 1 pmol linearized, sterile targeting construct.

Electroporated cells were then plated cells in ES medium at approximately 2×10⁶ cells per 100-mm gelatin-coated tissue culture plate and incubated for 24 hr. Following this cells were selected for the presence of the selectable marker/s using neomycin and/or zeocin. Selection was continued until single, isolated colonies were visible.

Cells were then screened to determine whether or not the targeting construct has integrated by homologous recombination. ES cells were incubated in 24-well microtiter plates to near confluence (usually 2 to 3 days). Cells were then lysed and genomic DNA isolated. Correct integration of the targeting construct into the genome was determined using Southern blot analysis using a 1.2 kb BstElI/EcoRI fragment of the mouse dynorphin gene. This fragment hybridizes to a site located 5′ to the dynorphin gene upstream of the targeting construct.

Blastocysts were then isolated. Approximately one week prior to the day of ES cell injection 3 to 4 week old female mice were injected intraperitoneally with 0.1 ml of 50 U/ml PMS (5 U) to induce superovulation. At 46 to 48 hr after the PMS injection, each female is injected intraperitoneally with 5 U of 0.1 ml of 50 U/mI HCG (5 U).

Female mice were then mated. On post-coital day 3.5, blastocysts with an intact zona pellucida were collected. ES cells prepared previously were then microinjected into the collected blastocysts.

Blastocysts were then incubated for a time sufficient for them to recover from the injection of the ES cells. The blastocysts were then injected into the uterine lumen of a pseudopregnant female mouse.

Pups born from the injected females were screened using Southern blotting using the probe described supra to determine those in which the coding region of dynorphin has been removed. Chimeric knockout mice are then bred with wild-type mice and the offspring screened to determine whether or not they were heterozygous for the mutation (i.e. dynorphin^{+/-}). Heterozygous mice were then bred to produce homozygous dynorphin knockout mice (dynorphin^{-/-}).

### EXAMPLE 2

### Effects of Reducing Dynorphin Expression on Bodyweight, Body Fat Content, Appetite and Activity

### Animals

All research and animal care procedures were approved by the Garvan Institute/St Vincent's Hospital Animal Experimentation Ethics Committee and were in agreement with the Australian Code of Practice for the Care and Use of Animals for Scientific Purpose. Mice were housed under conditions of controlled temperature (22° C.) and illumination (12 hour light cycle, lights on at 7.00 h).

### Diets

Half of the mice of each genotype were fed a normal chow diet ad libitum (6% calories from fat, 21% calories from protein, 71% calories from carbohydrate, 2.6 kilocalories/g, Gordon's Speciality Stock Feeds, Yanderra, NSW, Australia). The other half was fed ad libitum with a high-fat diet supplemented with fat and sucrose (46% calories from fat, 21% calories from protein, 33% calories from carbohydrate, 4.72 kilocalories/g) from 4 weeks of age onwards. The diet was based on the composition of Rodent Diet Catalogue Number D12451 (Research Diets, New Brunswick, N.J.), with the exception that safflower oil and copha were used in place of soybean oil and lard.

Mice were also fasted to determine the effect of reduced dynorphin on fasting induced bodyweight, appetite levels and fat content. Mice were transferred to individual cages and allowed to acclimatize with ad libitum access to normal rodent chow and water for about 3 nights prior to experimentation. About half of the mice were then deprived of food for about 24 hours and the other half allowed ad libitum access to normal rodent chow.

### Measurement of Appetite and Bodyweight

Bodyweight was monitored at the same time each week from 4 weeks of age onwards. Food intake was measured over 3 days in individually housed mice at 11-12 weeks of age. At 12-13 weeks of age, mice were fasted for 24 hours for determination of fasting-induced weight loss. Upon completion of the 24-hour fast, food was reintroduced and food intake was determined 1, 2, 8, 24, 48, and 72 hours later. For all analyses of food intake, the amount of food spilled but not eaten was taken into consideration.

### Activity Measurements:

The total cage activity was recorded using a passive infrared detector (PID, Conrad Electronics, Germany) on top of the cage lid of single-housed animals. The signals of the PIDs were detected by an I/O interface card (PIO48 II, Germany) and stored by custom-made software. Due to the design of the PIDs the maximal number of counts per minute was 20. To show the activity pattern of the mice the data are plotted so that each cycle's activity is shown both to the right and below that of the previous cycle, a so-called double plotted actogram with the x-axis showing 48 hours starting on the left with 00:00 h (ZT 6). In order to test for differences in total activity between the genotypes the activity counts were summed up to 30 minute-bins and a mean/day was calculated by averaging corresponding bins of 7 consecutive days.

### Tissue Collection and Body Fat Measurement

At 14-15 weeks of age, mice were killed by cervical dislocation in the morning for collection of trunk blood. White adipose tissue (WAT) depots (right inguinal WATi, right epididymal WATe, right retroperitoneal WATr, and mesenteric WATm) were collected from cadavas and weighed. The sum of these adipose tissue depots were expressed as total white adipose tissue weight, WATt. The interscapular brown adipose tissue depot (BAT) was also collected and weighed.

### Statistical Analyses.

In the examples presented throughout, data are expressed as mean ±SEM. Differences between groups were assessed by factorial ANOVA or repeated measures ANOVA (for body weight, glucose tolerance results, and white adipose tissue mass). When there was a significant overall effect or interaction effect, Fisher's post-hoc tests were performed to locate differences, using StatView version 4.5 (Abacus Concepts Inc, CA). For all statistical analyses, P<0.05 was accepted as being statistically significant.

### Results

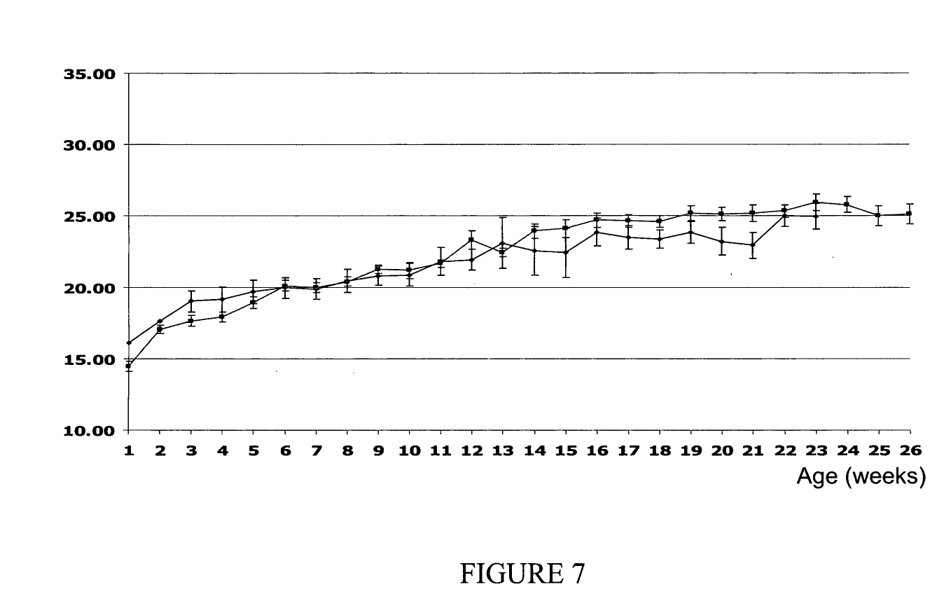

As shown in FIG. 7, reduced dynorphin expression had no significant effect on the bodyweight of mice fed on normal mouse chow.

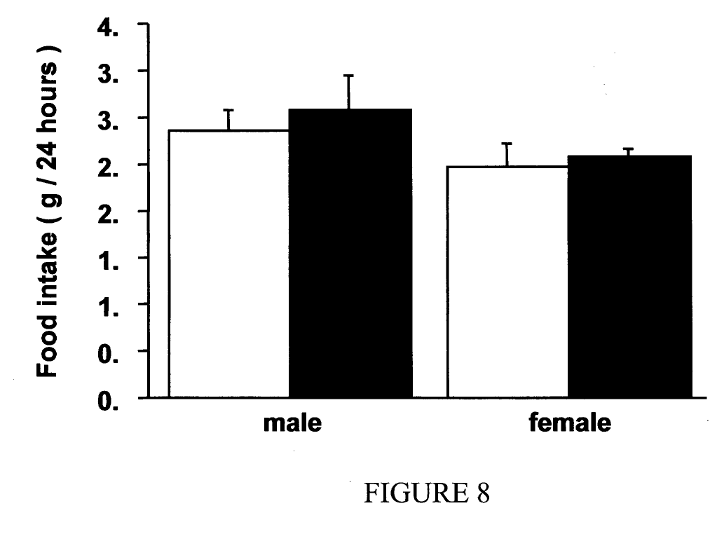

Furthermore, dynorphin^{-/-} mice consumed about the same amount of food as wild-type mice when fed on normal mouse chow ad libitum (FIG. 8).

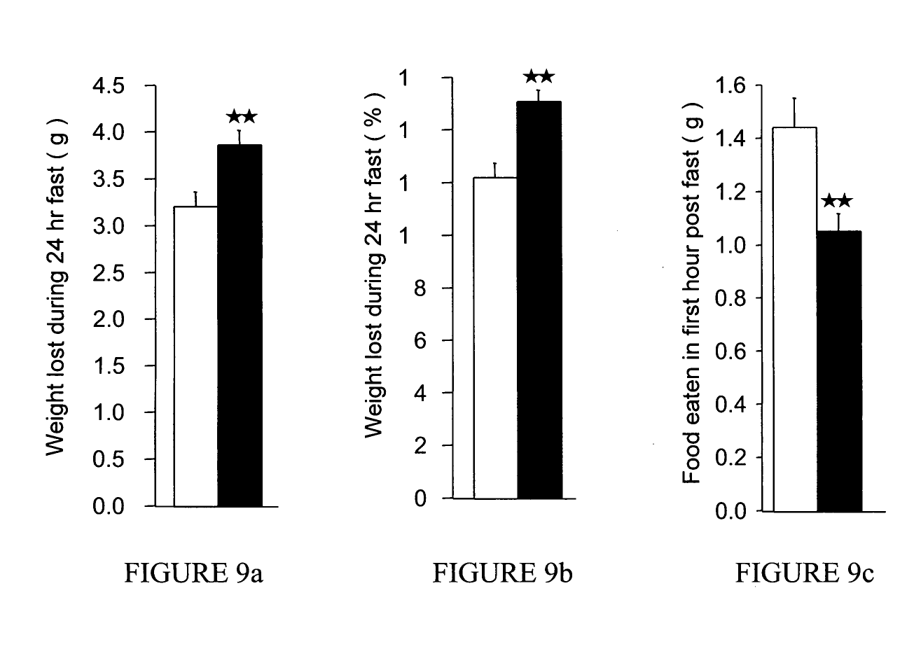

However, following a period of fasting dynorphin^{-/-} showed a significantly different response to wild type mice. In particular, during a 24 hour fast, dynorphin^{-/-} mice lost significantly more weight than wild-type mice (FIGS. 9a and 9b), losing approximately 2.5% more of their bodyweight than wild-type controls.

Furthermore, in the first hour after fasting, dynorphin^{-/-} mice consumed significantly less food than wild-type mice (FIG. 9c). This suggests that diet-induced appetite is suppressed in dynorphin^{-/-} mice.

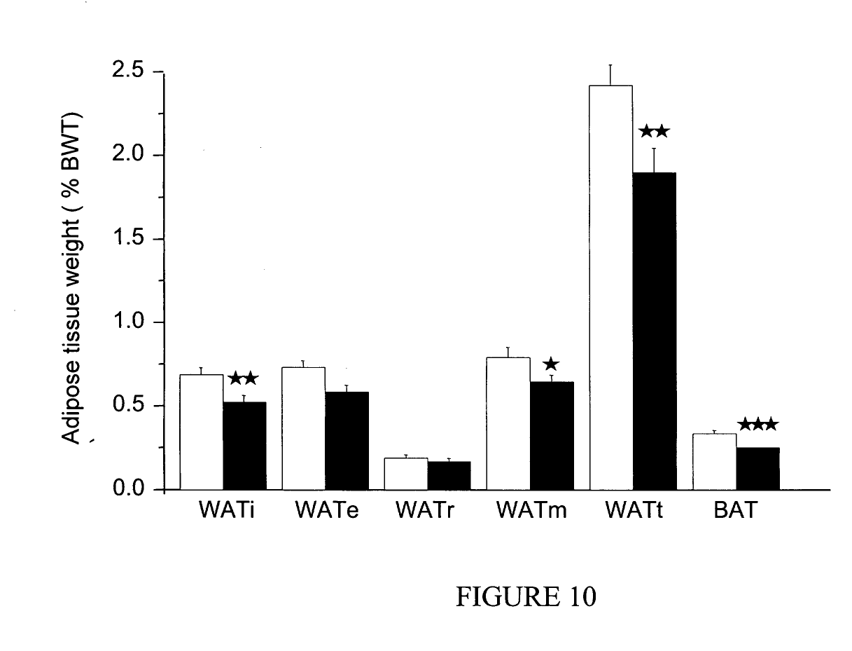

Despite the dynorphin^{-/-} mice and wild-type mice not differing in their bodyweight when fed normal rodent chow, the knockout mice had significantly lower fat content than controls (FIG. 10). For example, dynorphin^{-/-} mice had significantly reduced levels of fat in the inguinal and mesenteric fat pads (i.e., the fat pads were lighter). All of the changes in white adipose tissue weights led to a significantly reduced level of white adipose tissue in dynorphin^{-/-} mice.

Furthermore, as shown in FIG. 10 dynorphin^{-/-} mice had significantly reduced levels of brown adipose tissue compared to wild-type mice.

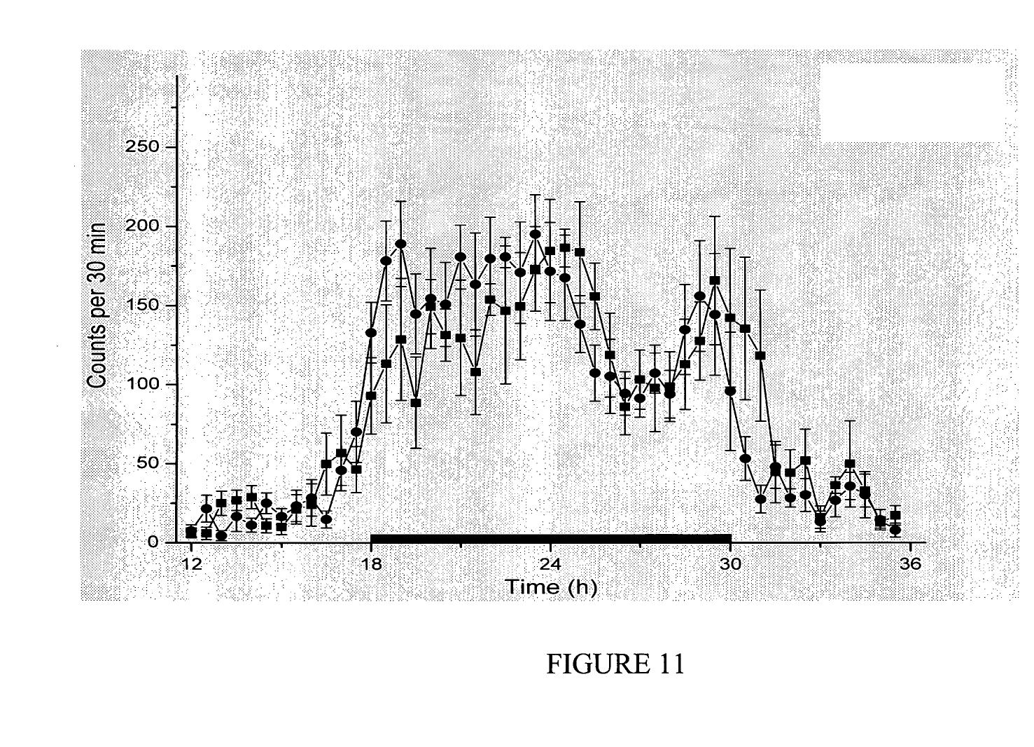

To investigate whether dynorphin deficiency might affect energy expenditure due to physical activity, mice were tested for general activity. Data attained indicate that dyhorphin^{-/-} and wild type mice are indistinguishable in the amount and pattern of activity (FIG. 11). These results suggest that locomotion is not altered in these mice.

### EXAMPLE 3

### Effect of Reduced Dynorphin Expression on Glucose Homeostasis

### Glucose Tolerance Test

At 13-14 weeks of age animals were fasted overnight and then administered an intraperitoneal injection of D-glucose (1.0 g / kg). Blood samples were collected from the tail at 0, 15, 30, 60, 90, and 120 minutes post injection for determination of serum glucose and insulin levels.

### Tissue Collection and Analysis.

At 14-15 weeks of age, mice were killed by cervical dislocation in the morning for collection of trunk blood. Serum insulin levels were measured by radioimmunoassay kits from Linco Research (St. Louis, Mo., USA) or an ultrasensitive enzyme immunoassay for post-glucose serum samples (Mercodia AB, Uppsala, Sweden).

### Results

To determine whether dynorphin ablation affects serum insulin levels and glucose tolerance, in vivo glucose tolerance tests were performed.

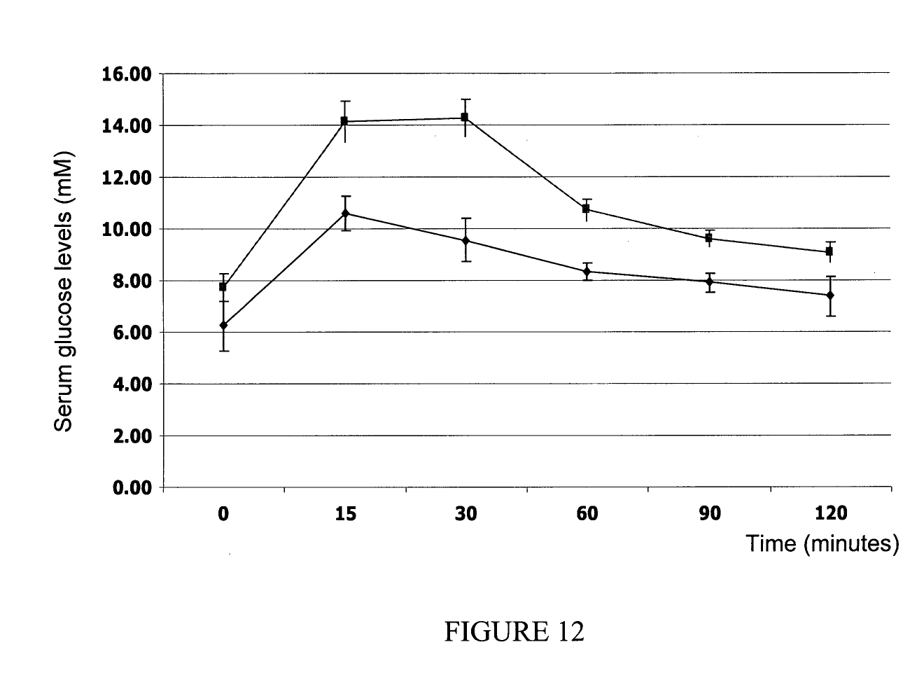

In chow-fed animals, dynorphin deficiency improves glucose tolerance. In male dynorphin^{-/-} mice the peak in glycemia was significantly lower than that of wild type mice (FIG. 12).

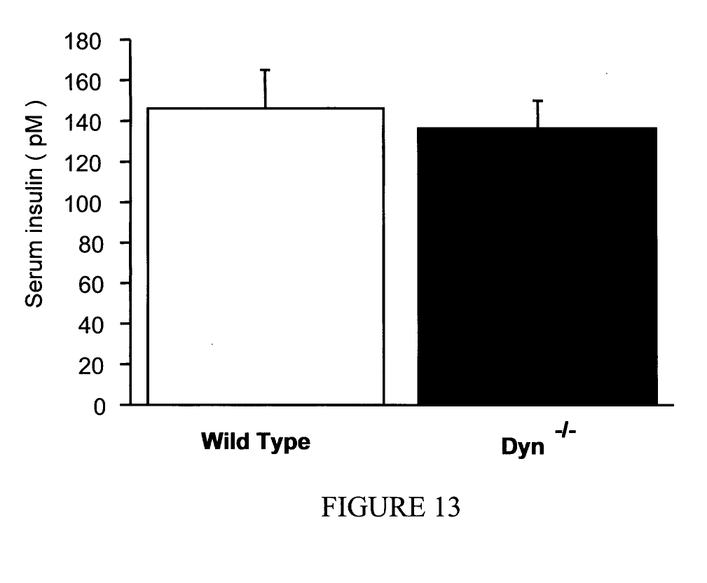

However, dynorphin deficiency did not alter basal serum insulin levels in either fed or fasted mice compared to wild-type controls (FIG. 13). Accordingly, the improved glucose tolerance of male dynorphin^{-/-} mice does not appear to be due to increased insulin secretion.

### EXAMPLE 4

### Effect of Reduced Dynorphin Expression on Diet Induced Obesity

### Animals and Measurements

Mice were maintained on a high fat diet and the effect on various parameters assessed essentially as described in Example 2.

### Results

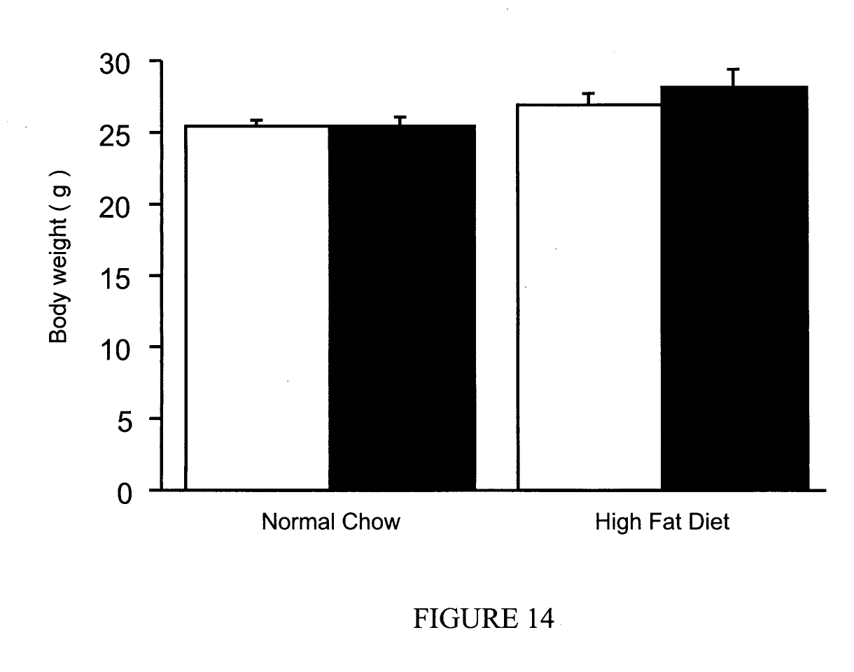

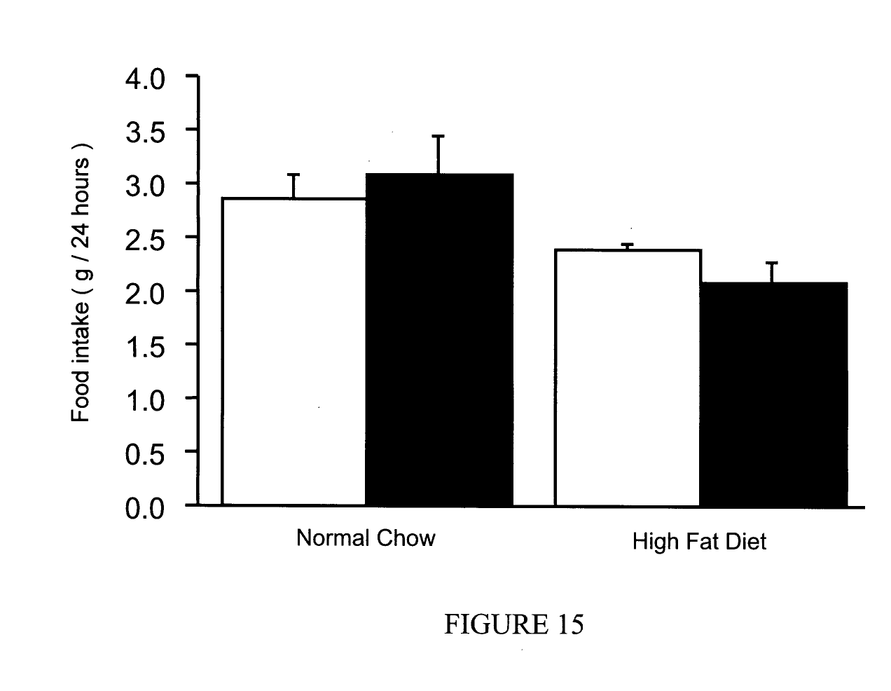

As shown in FIG. 14, reduced dynorphin expression had no effect on the weight of diet-induced obese mice compared to wild-type control mice. Furthermore, the food consumed by dynorphin^{-/-} mice fed on a high fat diet did not significantly vary from that consumed by wild-type mice fed on the same diet (FIG. 15).

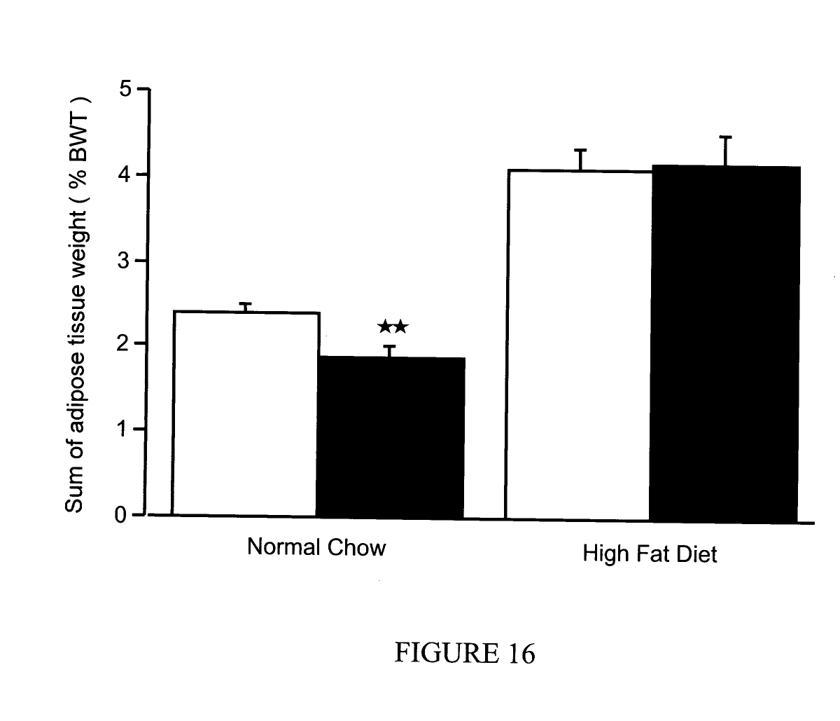

In contrast to dynorphin^{-/-} mice fed on normal chow, the body fat content of dynorphin^{-/-} mice fed on a high fat diet did not vary significantly from wild-type mice fed on the same diet. This is shown in FIG. 16 where the total adipose tissue weight from each mouse is expressed as a percentage of that mouse's bodyweight.

### EXAMPLE 5

### Effect of Reduced Dynorphin Expression on Serum Hormone Concentrations

Since dynorphin knockout reduces body fat levels in fasted animals, serum concentrations of IGF-1 were determined. IGF-1 is the main mediator of the growth effects of growth hormone.

### Animals

Mice were maintained on chow diets, high fat diets and fasted essentially as described in Example 2.

### Tissue Collections and Measurments

At 14-15 weeks of age, mice were killed by cervical dislocation in the morning for collection of trunk blood. Plasma levels of free T4 were measured with radioimmunoassay kits from ICN Biomedicals (Costa Mesa, Calif.). Plasma IGF-1 concentrations were radioimmunoassayed with a kit from Bioclone Australia (Marrickville, Australia). Glycemia was determined with a colorimetric kit (Trace Scientific, Melbourne, Australia).

### Results

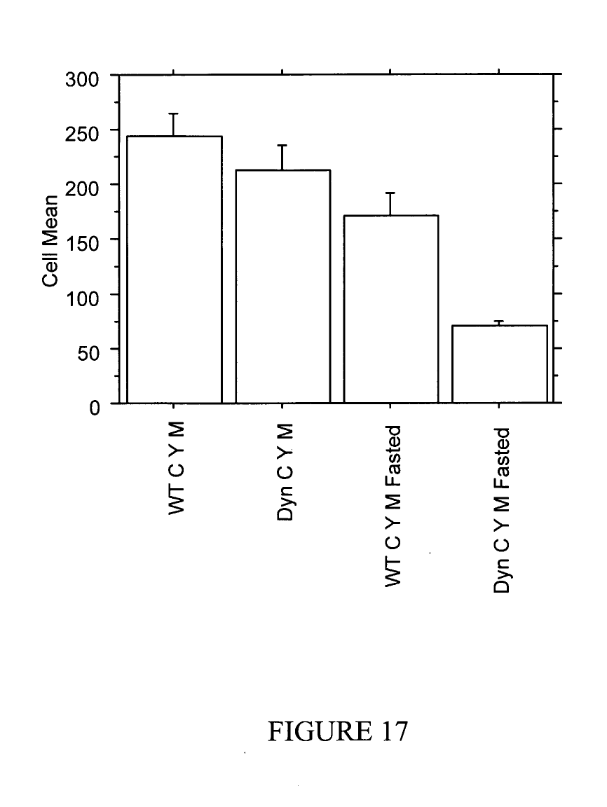

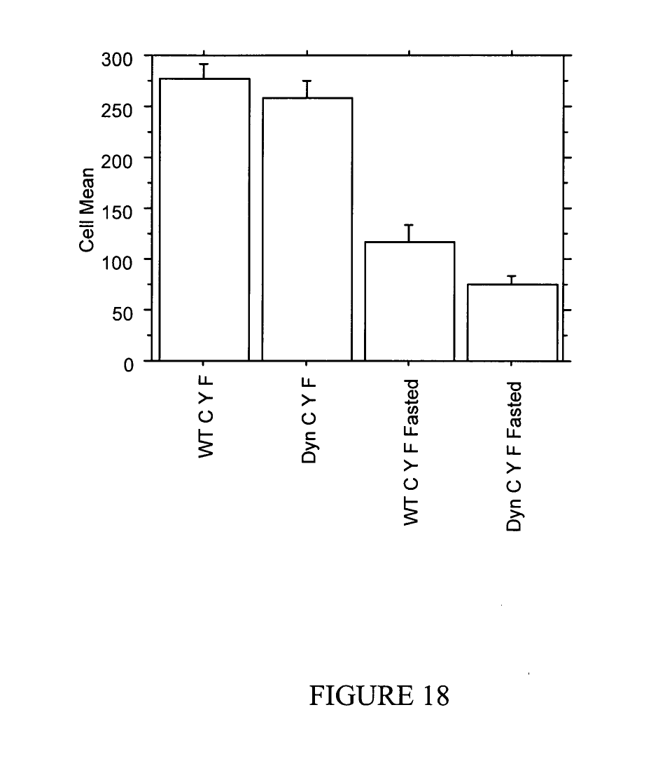

As shown in FIGS. 17 and 18 dynorphin^{-/-} males have lower IGF-1 levels than wild-type controls in the fed state.

Following fasting, IGF-1 levels are reduced in wild-type mice (both male and female). However, the reduction in serum IGF-1 levels in dynorphin^{-/-} mice is even greater than that observed in wild-type mice.

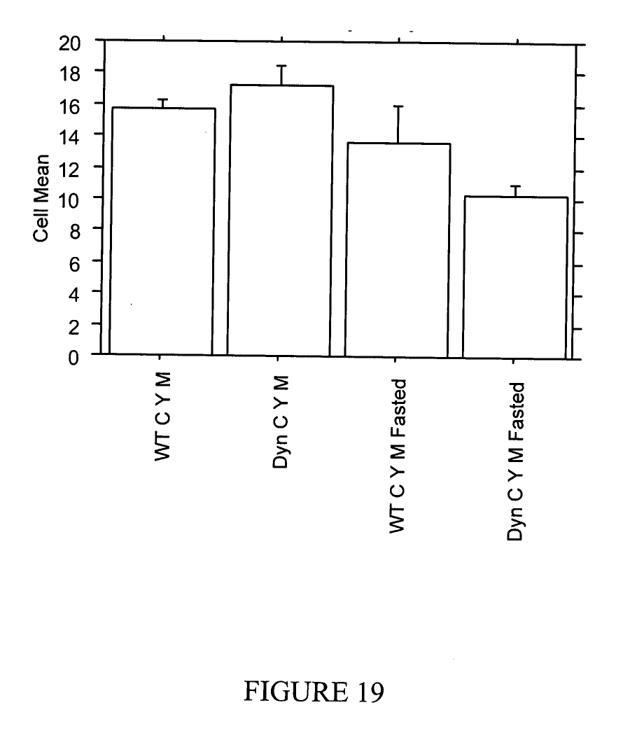

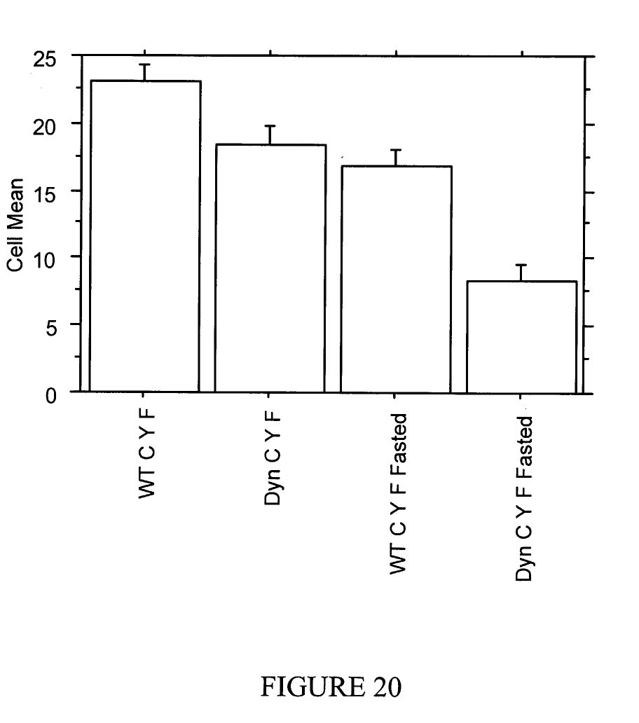

As an index of hypothalamo-pituitary-thyroid function (which is an important determinant of resting metabolic rate), serum free T4 levels was dynorphin knockout and wild type mice. As shown in FIGS. 19 and 20 dynorphin^{-/-} female mice have a significantly reduced level of serum T4 compared to wild-type controls in the fed state.

Following fasting, serum T4 levels are reduced in wild-type mice, consistent with the effects of fasting on this hormone. However, the reduction in serum T4 was greater in fasted female dynorphin^{-/-} mice compared to fasted wild-type mice.

### EXAMPLE 6

### Role of Dynorphins in Mediating Short-Term Starvation Responses

To determine the role of dynorphin in short-term starvation response hormonal and metabolic responses to 48-hour fasting in dynorphin knockout are compared to wild type mice.

In wild-type mice overnight to 48-hour fasting leads to decreases in body weight, total energy expenditure (EE), expression of uncoupling protein-1 (UCP-1) in brown adipose tissue (BAT), total body fat mass, weight of white adipose tissue (WAT) depots, serum glucose concentrations, and total body lean mass. These changes are at least partially mediated by decreased activity of the hypothalamo-pituitary-thyrotropic, -gonadotropic, and -somatotropic axes, and increased activity of the hypothalamo-pituitary-adrenal axis, resulting in corresponding changes in serum concentrations of the hormonal mediators / indices free T4, testosterone or estradiol, insulin-like growth factor 1, (IGF-1), and corticosterone. These changes are associated with altered expression of the hypothalamic regulators of these axes; thyrotropin releasing hormone, TRH; gonadotropin releasing hormone, GnRH; growth hormone releasing hormone, GHRH; corticotropin releasing hormone, CRH; and NPY. The magnitude of changes observed after 48-hour fasting are sufficiently marked to allow detection of potential differences between genotypes.

To determine the effect of dynorphin on short term starvation responses the following 8 groups of 16 week-old mice are investigated in either the fed state or after 48 hours of food deprivation as shown in Table 1:

TABLE 1
Mice used to determine the role of dynorphin in mediating short-term
starvation response

| Male mice | | | | female mice | | | |
|---|---|---|---|---|---|---|---|
| wild type | | Dyn^{-/-} | | wild type | | Dyn^{-/-} | |
| fed | fasted | fed | fasted | Fed | fasted | fed | fasted |
| (n = 12) | (n = | (n = 12) | (n = 12) | (n = 12) | (n = 12) | (n = 12) | (n = 12) |
| | 12) | | | | | | |

The parameters that are measured as an index of the starvation response are listed in Table 2.

TABLE 2
Parameters of energy homeostasis that are measured as an index of the
starvation response.

| Peripheral measures of | Hormonal | Hypothalamic | Hypothalamo- |
|---|---|---|---|
| energy balance | mediators | mediators | pituitary Axis |
| 24 & 48 hr weight loss | Basal insulin | — | — |
| 24 & 48 hour EE | Glucose- | | |
| BAT UCP-1 mRNA | induced ins. | | |
| Total body fat mass | Free T4 | TRH | thyrotropic |
| Weight of WAT | Corticosterone | CRH | adrenal |
| depots | | | |
| Serum glucose levels | Testosterone or | GnRH | gonadotropic |
| Glucose tolerance | estadiol | | |
| (IPGTT) | IGF-1 | GHRH | somatotropic |
| Total body lean mass | All of the | NPY | — |
| | above | | |

Mice are transferred to individual cages in a calorimeter (Oxymax®,Columbus Instruments, Columbus, Ohio, USA) and allowed to acclimatize with ad libitum access to normal rodent chow and water for at least 3 nights prior to experimentation. Half of the mice in the calorimeter chambers are of food for about 24 hours to about 48 hours, and the other half have continued ad libitum access to food.

### Total Energy Expenditure During the Fasting Period Is Measured.

Mice are weighed periodically during and after the fasting period to determine weight loss.

Animals are then sacrificed at a standard time for collection of trunk blood. Brains and brown adipose tissue are immediately removed and frozen on dry ice. Samples are rapidly frozen for subsequent determination of serum hormones and metabolites, expression of UCP-1 in BAT as an index of energy expenditure for thermogenesis, and hypothalamic expression of the peptide hormones in Table 1. Hormone and metabolite levels are determined as previously published.

Carcasses are subject to dual-energy x-ray absorptiometry (Lunar PIXImus2 mouse densitometer; GE Healthcare) for determination of total body fat and lean mass.

White adipose tissue depots will be dissected out and weighed essentially as described in Example 2, as an index of region-specific changes in adiposity.

Subsets of mice are subject to intraperitoneal glucose tolerance tests (IPGTT, 1.0 mg/kg) with subsequent measurement of serum insulin and glucose levels up to 2 hours post-glucose, as previously described in Example 2. These measurements indicate whether dynorphin deletion affects glucose tolerance, insulin sensitivity (by comparison of ratios of serum insulin:glucose), and insulin response to glucose.

Data are compared by analysis of variance (ANOVA) to determine the effect of fasting, dynorphin gene, and gender) and Fisher's post-hoc analysis where necessary.

### EXAMPLE 7

### Interaction Between Dynorphin and NPY in Regulating Energy Homeostasis

### Animals

Mice were maintained essentially as described in Example 2.

### Tissue Collection and Analysis.

At 14-15 weeks of age, mice were killed by cervical dislocation in the morning for collection of trunk blood. The brain was removed and immediately frozen on dry ice.

### In Situ Hybridization.

Coronal slices (20 μm) of fresh frozen brains were cut and thaw-mounted on charged slides. For radioactive in situ hybridisation, DNA oligonucleotides complementary to mouse NPY:

5′-GAGGGTCAGTCCACACAGCCCCATTCGCTT-GTTACCTAGCAT-3′ (SEQ ID NO: 8); and mouse dynorphin:

5′- TTCAGGACGGGTTCCMGAGC-TTGGCATGTGCACTGATGCCT-3′ (SEQ ID NO: 9) were labelled with [³⁵S] thio-dATP (Amersham Pharmacia or NEN) using terminal deoxynucleotidyltransferase (Roche, Mannheim, Germany or Amersham Pharmacia).

A set of brain sections from wild type mice was used to assay the distribution of dynorphin mRNA expression. Matching sections from the same portion of the arcuate nuceli (Arc) of knockout and wild type control mice were used to examine NPY mRNA expression and assayed together, as described previously (Sainsbury et al., Diabetes, 2002).

NPY and dynorphin mRNA expression levels were evaluated by measuring silver grain densities over individual neurons from photo-emulsion-dipped sections.

For evaluation of in-situ hybridization, digital images of the areas of interest were acquired from photo-emulsion dipped and superficially counter-stained brain slices at 200×magnification using a Zeiss Axiophot equipped with the ProgRes digital camera. Silver grain density was evaluated by an experimentally blinded observer by outlining single neurons and measuring the total neuronal area and the area covered by silver grains (black grains in brightfield image) using the NIH image software. Percentage of silvergrain area compared to total area calculated for single neurons was averaged. Data are provided as percent of control silver grain density averaged from at least 4 sections per peptide per animal.

### Dual Labelling In Situ Hybridisation.

Dual labelling in situ hybridisation was performed by combining both radioactive and non-radioactive in situ hybridisation protocols. For the radioactive in situ hybridisation, the mouse dynorphin DNA oligonucleotides were labelled with [³⁵S] thio-dATP as described above. For the non-radioactive in situ hybridisation, NPY DNA oligonucleotides were tailed with DIG at the 3′ end using terminal transferase (DIG Use's Guide Manual, Boehringer Mannheim, 1995). The mixture of hybridisation solution was as described above. The ³⁵S-labeled probe was diluted to 106 cpm/ml with the hybridisation solution, and the digoxigenin-labelled probe was used in the dilution of 2.5 pmol/ml. The hybridisation solution (100 μl) was then applied to each section and incubated for 16 hours at 42° C. The sections were washed twice in 2×SSC with 50% formamide and 100 μl 1 M dithiothreitol at 40° C. and twice in 2×SSC with 50% formamide at 40° C. for 15 min each, respectively. Following the washes, a standard colorimetric detection system employing NBT and X-phosphate as substrate was used to detect the dig-labelling NPY signals (Boehringer Mannheim, Germany). Sections were air-dried and dipped in 3% parlodion (Sigma) dissolved in diethyl ether. Slides were air-dried overnight, dipped in photographic emulsion (Kodak) and then stored in foil-wrapped slide boxes at 4° C. for two weeks. Slides were developed with D-19 developer (Kodak), and then immersed in dist water and cover-slipped with aquamount.

### Cell Counting

Cell counting was performed by using a grid reticule and the 10×objective of a Zeiss Axioplan light microscope. Double labelled cells within the arcuate nuclei were delineated through adjacent landmarks, according to the atlas of the mouse brain in stereotaxic coordinates by Franklin and Paxinos (Franklin and Paxinos, 1997). The positive double-labelling cells were counted if the number of silver grains overlying identified neuron bodies (digoxigenin-labeled) were 5 times above background hybridisation levels. The values represent an average of position neurons in a given area of one hemisphere from a single section. The average cell count in each nucleus was determined by both left and right sides, and then all groups were pooled for final analysis.

### Results

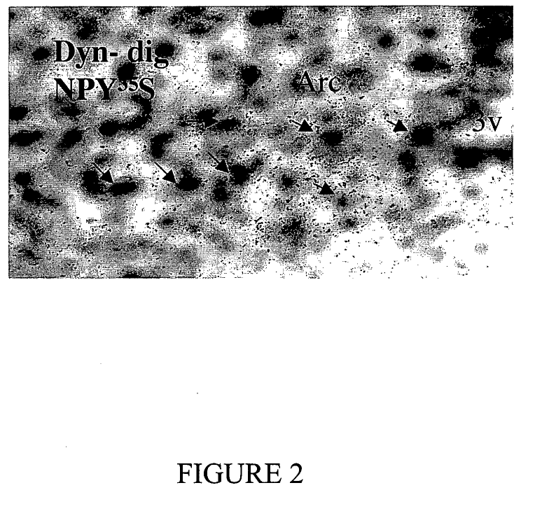

As shown in FIG. 2 pre-prodynorphin mRNA and NPY mRNA are co-localized to the regions of the hypothalamus. In fact, mRNA encoding pre-prodynorphin and NPY are expressed by some of the same neurons in the hypothalamus. In particular, both pre-prodynorphin and NPY are expressed by the region of the hypothalamus responsible for energy homeostasis, e.g., the arcuate nucleus.

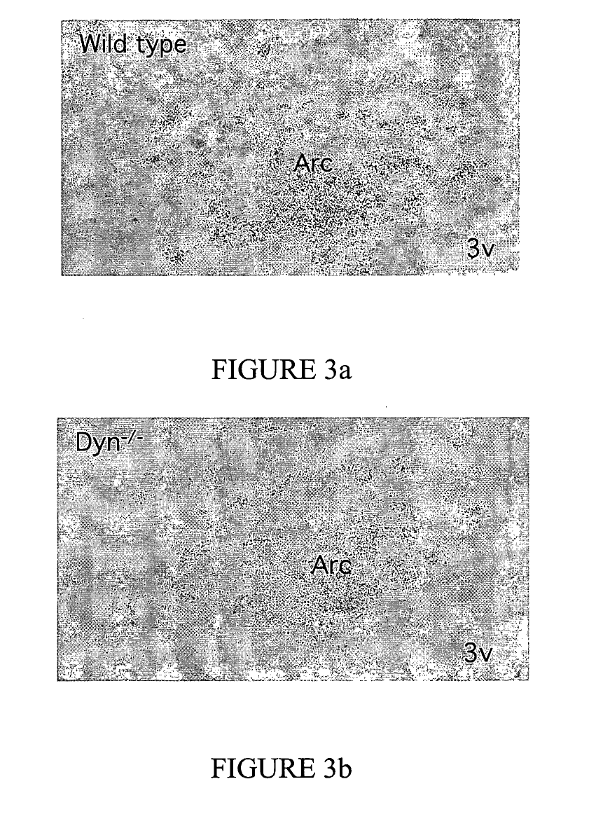

In the arcuate nucleus of dynorphin^{-/-} mice the expression of NPY mRNA is also reduced compared to the level of expression in wild type mice (FIGS. 3a and 3b, 77.6±3.7% of wild type values, mean ±SEM of 5-6 mice per group, P<0.01). These results indicate that one or more forms of dynorphin stimulate expression of NPY.

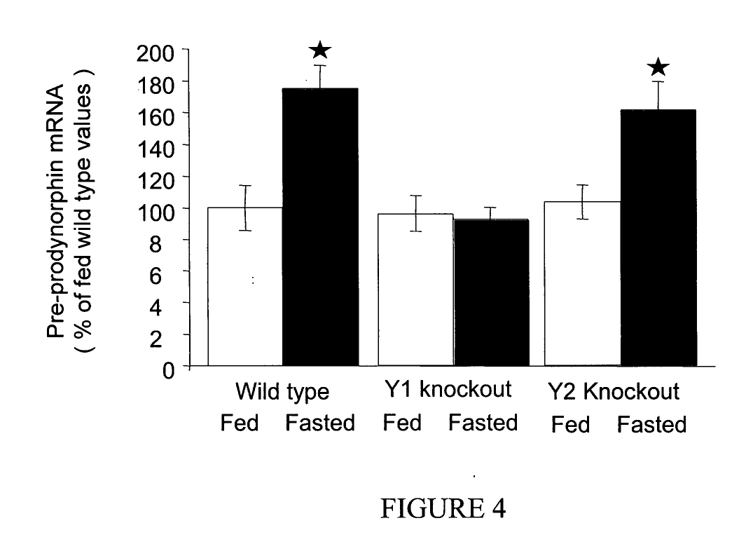

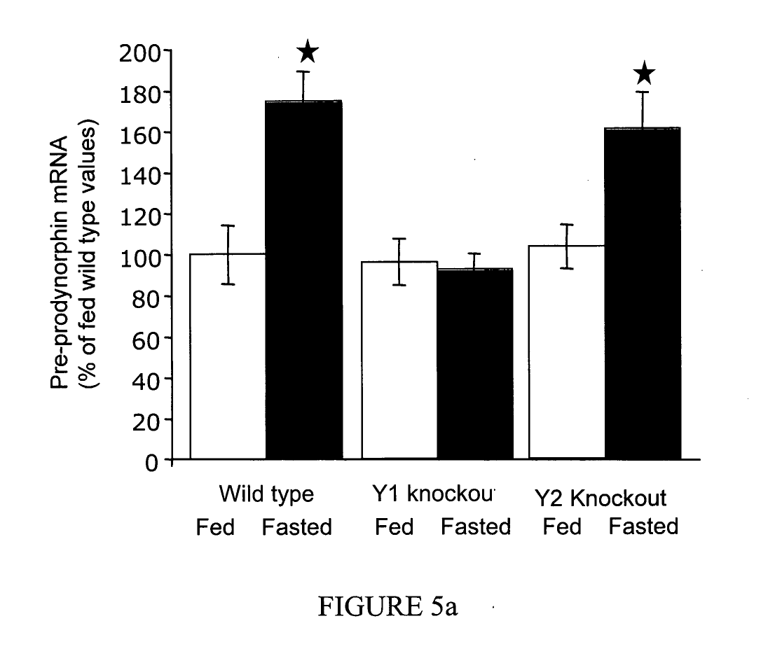

Furthermore, NPY also appears to be capable of modulating (stimulating) dynorphin expression. As shown in FIG. 4 fasting induces expression of dynorphin in wild-type mice to a significant degree. Similar results were obtained in Y2 receptor deficient mice, but not in Y1 receptor deficient mice.

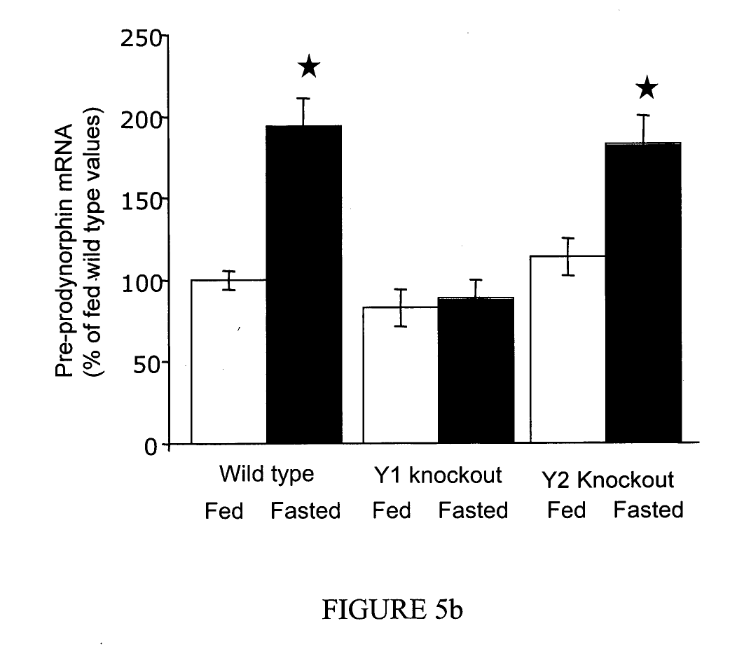

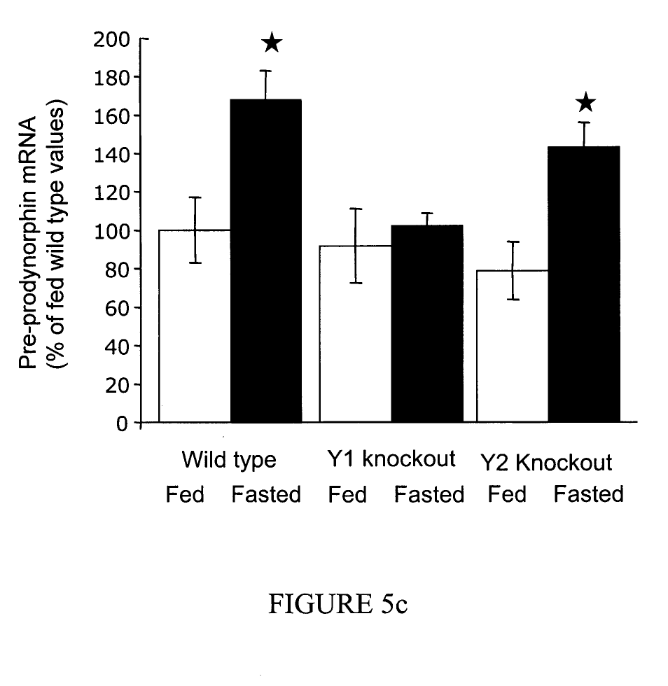

FIGS. 5a to c show the level of expression of dynorphin mRNA in various regions of the hypothalamus, in particular, the arcuate nucleus (FIG. 5a), the paraventricular nucleus (FIG. 5b) and the ventromedial hypothalamus (FIG. 5c). Again, fasting induced expression of dynorphin mRNA in wild-type and Y2 receptor deficient mice but not in Y1 receptor deficient mice. These results indicate that NPY stimulates expression of dynorphin via Y1 receptors and not Y2 receptors.

### EXAMPLE 8

### Interaction Between Dynorphin and NPY in Mediating Short-Term Starvation Defences

As NPY and dynorphin are expressed in the same area of the brain and have both been shown to have an effect on body weight/metabolism, the effect of reduced expression of both neuropeptides is determined.

The effect of fasting is determined in the following 8 groups of 16 week-old mice, using assays essentially as described in Example 6 (see Table 3).

TABLE 3
Mice used to determine the effect of dynorphin and NPY in mediating
short-term starvation defences

| male mice | | | | female mice | | | |
|---|---|---|---|---|---|---|---|
| NPY^{-/-} | | [Dyn^{-/-}NPY^{-/-}] | | NPY^{-/-} | | [Dyn^{-/-}NPY^{-/-}] | |
| fed | fasted | fed | fasted | Fed | Fasted | fed | fasted |
| (n = | (n = | (n = 12) | (n = 12) | (n = | (n = 12) | (n = 12) | (n = 12) |
| 12) | 12) | | | 12) | | | |

As the genes encoding NPY and dynorphin are located on separate Chromosomes, dynorphin^{-/-}NPY^{-/-} mice are produced by cross breeding mice homozygous for either mutation and offspring interbred.

Data from Each Gender Are Analyzed by ANOVA and Fisher's Post-Hoc Analysis where Necessary

These assays determine whether or not the assayed parameters are more pronounced in [Dyn^{-/-}NPY^{-/-}] than in Dyn^{-/-} or NPY^{-/-} mice, indicating that dynorphins and NPY signal, at least partially by distinct (e.g., additive) mechanisms.

### EXAMPLE 9

### Interaction Between Dynorphin and Leptin Deficiency in Mediating Long-Term Starvation Defences

To determine whether or not dynorphin is involved in maintaining the chronic starvation-like neuroendocrine responses of leptin-deficient ob/ob mice, the effect of dynorphin deficiency on the phenotype of ob/ob mice is assayed.

In this study, the following 8 groups of adult mice are analysed as shown in Table 4.

TABLE 4
Mice used to determine the effect of dynorphin and leptin in mediating
long-term starvation defences

| male mice | | | | female mice | | | |
|---|---|---|---|---|---|---|---|
| lean | | ob/ob | | Lean | | ob/ob | |
| background | | background | | background | | background | |
| wild type | Dyn^{-/-} | ob/ob | [Dyn^{-/-}ob/ob] | Wild type | Dyn^{-/-} | ob/ob | [Dyn^{-/-} |
| (n = 12) | (n = 12) | (n = 12) | (n = 12) | (n = 12) | (n = 12) | (n = 12) | ob/ob] |
| | | | | | | | (n = 12) |

As the genes encoding pre-prodynorphin and leptin (ob) are located on separate mouse chromosomes, [Dyn^{-/-}ob/ob] double knockout mice for these experiments are produced by interbreeding the mouse of the invention and ob/ob mice.

At 15 weeks of age mice are transferred to individual cages in the Oxymax® calorimeter and allowed to acclimatize with ad libitum access to normal rodent chow and water for at least 3 nights prior to experimentation. Total 24-hour food intake, energy expenditure, and physical activity are determined in the ensuing 48 hours and the average of 2 day's measurements made for each animal. The actual amount of food eaten is determined as the amount of food removed from the cage hopper minus the amount of food spilled in the bottom of the cage. Thereafter, conscious mice are subject to IPGTT as described above. Following recovery from IPGTT (at least 1 week), mice are culled and tissues collected and analyzed in accordance with the procedure above. Data are analysed as described above. Mice are analysed to determine whether or not a reduction in dynorphin expression rescues or normalizes the ob/ob obese phenotype (i.e., causes these mice to become leaner).

1. A transgenic non-human animal in which the expression of one or more dynorphin peptides is reduced or disrupted.

2. (canceled)

3. (canceled)

4. (canceled)

5. (canceled)

6. (canceled)

7. The transgenic non-human animal of claim 1 further comprising one or more additional modifications to its genome.

8. The transgenic non-human animal of claim 7 wherein the additional modification comprises the knockout of an NPY-encoding gene.

9. The transgenic non-human animal of claim 7 wherein the additional modification comprises the knockout of one or more NPY receptor-encoding genes.

10. (canceled)

11. The transgenic non-human animal of claim 7 wherein the additional modification comprises the knockout of a leptin gene.

12. (canceled)

13. The transgenic non-human animal of claim 1 comprising a genetic background that confers an obesity or diabetes syndrome and the expression of one or more dynorphin peptides is reduced or disrupted in said genetic background.

14. (canceled)

15. A progeny animal of the transgenic non-human animal of claim 1 wherein said progeny animal is deficient in expression of at least one allele of a preprodynorphin-encoding gene.

16. An isolated cell or tissue of the transgenic non-human animal of claim 1.

17. An isolated cell or tissue of the progeny animal of claim 15.

18. A method of identifying a dynorphin-mediated phenotype, said method comprising comparing a phenotype of a wild-type animal to a phenotype of a genetically modified non-human animal comprising a genetic modification within an allele of its preprodynorphin locus wherein said genetic modification reduces or prevents expression of a functional dynorphin peptide in said animal, and wherein a modified or different phenotype is a dynorphin-mediated phenotype.

19. (canceled)

20. (canceled)

21. A method of producing a dynorphin-deficient animal comprising introducing a vector comprising a nucleotide sequence set forth in SEQ ID NO: 7 or complementary thereto into the genome of a non-human mammalian cell for a time and under conditions sufficient for homologous recombination to occur thereby deleting at least about exons 3 and 4 of a preprodynorphin-encoding gene from said genome and regenerating a whole animal there from.

22. (canceled)

23. A vector comprising a nucleotide sequence set forth in SEQ ID NO: 7 or complementary thereto.

24. A method of identifying a compound that modulates a dynorphin-mediated phenotype of an animal, said method comprising: (a) administering a compound to a non-human animal expressing a functional dynorphin peptide and determining a dynorphin-mediated phenotype of the animal; and (b) comparing the dynorphin-mediated phenotype at (a) to the same phenotype of genetically modified non-human animal to which the compound has not been administered, said genetically modified non-human animal comprising a genetic modification within an allele of its preprodynorphin locus, wherein said genetic modification reduces or prevents expression of a functional dynorphin peptide in said animal, and wherein a comparable phenotype indicates that the compound modulates a dynorphin-mediated phenotype of the animal.

25. (canceled)

26. (canceled)

27. (canceled)

28. (canceled)

29. (canceled)

30. (canceled)

31. (canceled)

32. A method of identifying a compound that modulates a dynorphin-mediated phenotype of an animal, said method comprising: (a) administering a compound to a genetically modified non-human animal comprising a genetic modification within an allele of its preprodynorphin locus wherein said genetic modification reduces or prevents expression of a functional dynorphin peptide in said animal; and (b) determining a phenotype of the animal relative to the phenotype of an isogenic animal to which the compound has not been administered wherein a different phenotype indicates that the compound modulates a dynorphin-mediated phenotype of the animal.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. The method according to claim 24 said method comprising: (a) determining a compound that binds to a dynorphin receptor and/or reduces the dynorphin-mediated activation of a dynorphin receptor and/or produces a dynorphin-mediated change in intracellular calcium concentration; (b) administering a compound to a non-human animal expressing a functional dynorphin peptide and determining a dynorphin-mediated phenotype of the animal; (c) comparing the dynorphin-mediated phenotype at (b) to the same phenotype of genetically modified non-human animal to which the compound has not been administered, said genetically modified non-human animal comprising a genetic modification within an allele of its preprodynorphin locus, wherein said genetic modification reduces or prevents expression of a functional dynorphin peptide in said animal, and wherein a comparable phenotype indicates that the compound modulates a dynorphin-mediated phenotype of the animal.

51. The method according to claim 32 said method comprising: (a) determining a compound that binds to a dynorphin receptor and/or activates a dynorphin receptor and/or produces a change in intracellular calcium concentration; (b) administering a compound to a genetically modified non-human animal comprising a genetic modification within an allele of its preprodynorphin locus wherein said genetic modification reduces or prevents expression of a functional dynorphin peptide in said animal; and (c) determining a phenotype of the animal relative to the phenotype of an isogenic animal to which the compound has not been administered wherein a different phenotype indicates that the compound modulates a dynorphin-mediated phenotype of the animal.

52. (canceled)

53. (canceled)

54. (canceled)

55. (canceled)

56. The method according to claim 32 for identifying a compound that enhances feeding behaviour in a fasted animal, said method comprising: (a) administering a compound to a genetically modified non-human animal, said animal comprising a genetic modification within an allele of its preprodynorphin locus wherein said genetic modification reduces or prevents expression of a functional dynorphin peptide in said animal; and (b) determining the feeding behaviour of the animal, wherein enhanced appetite or dietary intake of the animal compared to the appetite or dietary intake of a preprodynorphin-deficient animal to which the compound has not been administered indicates that the compound enhances feeding behaviour in a fasted animal.

57. The method according to claim 24 for identifying a compound that reduces feeding behaviour during dieting or starvation, said method comprising: (a) administering a compound to a non-human animal expressing a functional dynorphin peptide; and (b) comparing the feeding behaviour of the animal to the feeding behaviour of a genetically modified non-human animal comprising a genetic modification within an allele of its preprodynorphin locus wherein said genetic modification reduces or prevents expression of a functional dynorphin peptide in said animal, wherein a comparable feeding behaviour indicates that the compound reduces feeding behaviour during dieting or starvation.

58. The method according to claim 32 for identifying a compound that enhances adiposity, said method comprising: (a) administering a compound to a genetically modified non-human animal comprising a genetic modification within an allele of its preprodynorphin locus wherein said genetic modification reduces or prevents expression of a functional dynorphin peptide in said animal; and (b) determining the fat content of the animal, wherein enhanced fat content of the animal compared to the fat content of a preprodynorphin-deficient animal to which the compound has not been administered indicates that the compound enhances adiposity.

59. A method of identifying a compound that reduces adiposity, said method comprising: (a) administering a compound that enhances fat deposition or glucose uptake to a genetically modified non-human animal comprising a genetic modification within an allele of its preprodynorphin locus wherein said genetic modification reduces or prevents expression of a functional dynorphin peptide in said animal and determining the fat content of the animal; (b) administering a test compound to the animal and determining the fat content of the animal, wherein a similar or reduced fat content at (b) compared to (a) indicates that the compound reduces fat deposition.

60. The method according to claim 24 for identifying a compound that reduces adiposity comprising: (a) administering a compound to a non-human animal expressing a functional preprodynorphin protein and determining the fat content of the animal; and (b) comparing the fat content at (a) to the fat content of a genetically modified non-human animal comprising a genetic modification within an allele of its preprodynorphin locus wherein said genetic modification reduces or prevents expression of a functional dynorphin peptide in said animal, wherein a comparable fat content indicates that the compound reduces adiposity.

61. The method according to claim 34 for identifying a compound that enhances glucose tolerance, said method comprising: (a) administering a compound to a genetically modified non-human male animal comprising a genetic modification within an allele of its preprodynorphin locus wherein said genetic modification reduces or prevents expression of a functional dynorphin peptide in said animal; and (b) perfonning a glucose tolerance test on the animal, wherein reduced serum glucose content of the animal compared to the serum glucose content of an isogenic male animal to which the compound has not been administered indicates that the compound enhances glucose tolerance.

62. A method of treating obesity in a subject comprising administering to the subject a compound that antagonizes the action of a dynorphin peptide in the subject.

63. A method of reducing appetite or food intake during dieting or fasting of a subject comprising administering to the subject a compound that antagonizes the action of a dynorphin peptide in the subject.

64. A method of increasing the reduction in body weight during dieting or fasting of a subject comprising administering to the subject a compound that antagonizes the action of a dynorphin peptide in the animal.

65. A method of reducing glucose intolerance of a male subject comprising administering to the subject a compound that antagonizes the action of a dynorphin peptide in the subject.


SEQUENCE LISTING (9 sequences)

SEQ ID NO: 1 - protein, 248 aa, Mus musculus
MAWSRLMLAACLLVMPSNVMADCLSLCSLCAVRIQDGPRPINPLICSLECQDLVPPSEEWETCRGFSSFLTLTVSGLRGKDDLEDEVALEEGISAHAKLLEPVLKELEKSRLLTSVPEEKFRGLSSSFGNGKESELAGADRMNDEAAQGRTVHFNEEDLRKQAKRYGGFLRKYPKRSSEMARDEDGGQDGDQVGHEDLYKRYGGFLRRIRPKLKWDNQKRYGGFLRRQFKVVTRSQENPNTYSEDLDV

SEQ ID NO: 2 - protein, 307 aa, Homo sapiens
MAWQGLVLAACLLMFPSTTADCLSRCSLCAVKTQDGPKPINPLICSLQCQAALLPSEEWERCQSFLSFFTPSTLGLNDKEDLGSKSVGEGPYSELAKLSGSFLKELEKSKFLPSISTKEEEKFRGLSSSFGNGKESELAGADRMNDEAAQGRTVHFNEEDLRKQAKRYGGFLNTLSKSLEEKLRGLSDGFREGAESELMRDAQLNDGAMETGTLYLAEEDPKEQVKRYGGFLRKYPKRSSEVAGEGDGDSMGHEDLYKRYGGFLRRIRPKLKWDNQKRYGGFLRRQFKVVTRSQEDPNAYSGELFDA

SEQ ID NO: 3 - DNA, 2400 bp, Mus musculus
  CDS (204)..(947): cDNA encoding mouse preprodynorphin
tggtagccctcgtcgcttcctctgtggcacttctctgagctagagtcagggtaccttctgaatcttggatcggccaccctaccacctgatcagccagaagcctgccagggacaaagcagtaagcaggtcattcatccccaggctttgagatctgcgtgggggagctcttgaagcagccccagcaggcaaacaagtgagtcagaatggcgtggtccaggctgatgctggcagcttgcctcctcgtgatgccctctaatgttatggcggactgcctgtccctgtgctccctgtgtgcagtgaggattcaggatgggccccgtcccatcaaccccctgatttgctccctggagtgccaggacctggtgccgccctcagaggagtgggagacatgccggggcttctcatcttttctcaccctgacggtctctgggctccgtggcaaggatgacttggaagatgaggttgctttggaagaaggcatcagtgcacatgccaagctcttggaacccgtcctgaaggagctggagaaaagccgactccttaccagcgtcccagaggaaaagttcaggggtctctccagcagctttggcaacggaaaagaatctgagctggcgggtgctgaccggatgaatgatgaagccgcacaggggcgcaccgtccattttaatgaggaggacttgagaaaacaggccaaacgctatggcggctttttgcgcaaataccccaagaggagttccgagatggcccgggatgaggacgggggccaggatggggatcaggtagggcatgaggacctgtacaaacgctatgggggcttcctgcggcgcattcgccccaagctgaagtgggacaaccagaagcgctatggtggtttcctgcggcgtcagttcaaggtggtgacgcggtcccaggagaaccccaatacctattctgaagatttagatgtttgatgcataacacttcccctccatggagttgggtctgcccccttccttgtactagaaccccctgcttcatcctcttgtgcttgttccgctgcttgctccattcagcactcccatgagaacgtctaagagcatcccacccctgttctgcttgttttcaggcaggggcagggaagagtggagatcccctgccaataggcccagggtccggctcaaaccccaagacctctaaactaccactactagtggctttttgacttcagcgtaaccgctctgtccctgtgatccctgcccatcccagaatccagagaaacttgttctttattcctatacgtctgtggtttccaaacttcactgaacgtgtgctcctctcggaagaaaaatgagcacttcctggtagtattgcatgggaactgtgagatatgcagtgtagaaacataaaaccagatacatgcaaatgaggatcctaactatattctcagccacagaggctcttcttatgcaccctaccctggaggccacaggataaatgaaaagccttttcaaggacagggattataaccctagagaggctgggcctctagcatgctaattgtctgtacacccagagtcctcatagtttcaaacgtttcctgaaaaacccagctcctagaccccggttagggcagtacctttccaagggtgaaatccccttgcttatgatgagacgccatccttctgaaggctgaagtgataaggggggagcaaagcaaactaatttgttctgtacaacaaattcagaatttgggccagttcccacagccctcattaaaaccgatcagtatcacacttctagcccatgaggaactgcatcggagtggagctgatgcggctgggcacagctgtgctcttcagtagtcgagttggctgcgggagccggagccatttggctgagccatttggcccagcgtatgcactggcactgggaaggggatccagaagcggtagctgaaggagagactgtcccgcctcaaaactacagcaagtctcattctctctgtctccaaataaaagcagaggaagaagaagacaacctgagtgttcaggatcaagtggattcgctggattcaagtaacccagagtcagagacaaggtcctctttggtttcacctccccatctccaggcagatatttagatgatgagttaacgggatggggctggacaggagaggaagcagactggatttgtatatttgcataagatatcttgagtgtccctctccacacacaggtcacagcctgtagagcccttcatgtgttctgaaatgtgtagattgttttacggtgacttgtgtgctgcttaaaaatcacatttatccaacctacttgggacatgtgatccttaataggtctgtgtagtttcctgcttctctggtataataaataaagttatgcggatggaaaaaaaaaaaaaaaa

SEQ ID NO: 4 - protein, 248 aa, Mus musculus; identical to SEQ ID NO: 1

SEQ ID NO: 5 - DNA, 15660 bp, Mus musculus
gctgacaaactctccacagcattgcttgtctaaggggaggatatggtgaaacaagtgacaacgtctgtgatgaaccaggtactcaccaaatagccctgtctgtcaccctagttgtatgagagcacgcaatgatccagcggcaggaagaggatgatgccccagagaagtacaaagtgtggctggtacatcagagcctgatgacgtaatcaccattacctggaggacagctgtagggaagtcctggccacatgccagctatggcaattgcttttggcatttcctcctttcactatgccaagatcccagtggggttctttcccccattctacagataagcagattgaggctgaacccactcacttgacttagggctctgcacagatgggtggcagaggcagagaccaagctgacacatccaatgttactactagctgtagctgtcatcgaccttgttcaggtaaacagatcaagtcacagacactgtagaaccaagtcctcagagtgggcgtttgtccagtagatgatgggaaggaccagatccatctaggtctgggtgatgacttgctgtgttctgtgtccagtttctaagctctccagcctctgcctctgcgcctggccagaatgtgaggattaaagttgatgtctttgctcactgcctggtgcaacccaggtgttcatgacacggactgcccagccagcctctgctatccccccccccccacaaggtgaacatctctgcagctgactacagagacaagcatgtttccttccaaactcagagacatgggtattctgggaggtgtttgcagccatccttgtttgtgagaaccaggggctgccagagaaaactgaatattgaacggcagcctctggttgaggaaaggcagtgtctggcatcaactcagacaggtgcccaagtcacctggcactctcaggcaaatgtacactcatatgcatgctagtgtgtgtgtgtgtgtgtgtgtgtgtgtgtgtgtgtgtgtgtgtccctgccaacatggagctgaatgttcctcttctataaccaagacctctgctttgaacacttcagcatatcctttggttccctatattttgagacagcctttgtttcttgcattcctcctcctccgaccccctgttcctctttttctcctgatctttgatagcctctgtggctgtggctctttctcttttcctcttactttgcctaggcttccttgtgcttcagacatgcctctctctgtttccattctctgattctcaacttttttttgcctttctattggactctctgcgcctctctctgtctcctcctatctctgcctttcccccacctttgtctctgttttcctgtgacactctcctggagatgctgaaaagtggccgcattgaaagtgacaaacagcgctacaccagaatccacccccgggcttcgcgaaagcatcctcctccacacccacatcaccaggagcagctattgtgtctcactcgggcccagggagttgagtggaaggacccgggggtctgagcgttgactgctctgccactcccccattggctcctggcagcctgtgctctgtgagcgagcgctgagagggctgagcgacagggggagactctcatctataaaaggggaataaggctccagaactgccatagggggatttggtagccctcgtcgcttcctctgtggcacttctctgagctagagtcagggtaccttctgaatcttggatcggccaccctaccacctgatcagccagaagcctgccagggacaaagcagtaagcaggtaagatccgcagggcgctcaggctctctggctttatgcatgtgctcctggtgctatgcaggggcgccggtgggagcttggacgggggaagttaggtttgggtctggttactcagcacagggaaatcatagtttcaaggtgagcttacagaagctcagagagtccaaagagctgcctgtcctctggaaaccatgccctgaacagtcctggcgcccacaggtgctagcggggcttcgcctctgccacacagcagtccccacactagttttcaggatctgaggagaaggtggtagcctgagtggaaaactgagaggtttcactaattagcatgatcttaaaggtcaacgttctcactggacttagcttacacaggttgggaacgttaatgaggttcaggactcggcatttgccaagtaccgatctgcaccatcaatgcataaataagtgccgtcctttttcactctttttgaatcacgtttaatctctgacaaggtgctgatttgagtggcttcctttgatttcactcagaatgaagtctggggttgtttggttgaaagggaactagaaaggaaagcaagagctcggctggatacccagtctgagggggacttcccatgtcaatttcactgtgaaaggacttgttgctttcctgtaaatcacagtcacgctaccgctggaggcagctaagttaggctggtggaggctcttttgaggaaagtcacccgctctctgatagcaatgctcatgtttaaagaaagtgctgtagaaccccaaagtttgacatcagcaaaaagctggcgagcttcagagacggcgactgtagtcacctccatgattgaacagtaaattctgtgtcttgtcacttttccctgtgtgtgtgtgtgttttctgagaacctctcaagatttacaagtatcccttaataaagcctccgagctctttaggttattggatctggtttcgagtaccctcccacagagagcaaaccttcattatcacccttctgccaagggttggtgaaatacacactgatatctttctaacctgacaggcgttgctgtcaagatctgttgcaaaacctctcagaatgtcagtccttttctgttcctagtcatgggggtttcatgcctacgctctgctcaccctcctcctctgctcacctcaccagtgctcccataccaccccactttatcttttgttctgtttttacaggtcattcatccccaggctttgagatctgcgtgggggagctcttgaagcagccccagcagggtgagttatgtgtgttgtgtgtgcatgtgtgtgcgtgtgtattggcataatcctgagcccactgctttgtcctcattgtgctaagtgtgatgggtggcaggtgactcctcttgtctccaggagtagctggcagatgggagtgcggtgtcacaatgggtccacggatgctgtgtggtaggatccactgttttgacaccgtaggctactcaagcggttgggtcagggctgtttgagaagacacgtggaagggtgtgataactctcaggacttccggagctttacctcaagaattttccatgtattgcctaagtgtgtgtgacccctcctgagcctgtgctgagttcaaacccaatggtcttccattatttgctgcgtgtaactttgtaacgactgcttttcctttttcagccgtaatctcctcaccaatataatgaagataataataatgtcctttccctatgtcactacaagactcctatgggaggctgtatgcgcctggcatttggtaggcactcagtaaatgctattatggctacctctctgatgactcaaaatggttggcttcaaaatgaagccctcccctctcttataaggcatcgtcagtcatcccattccatcctgctgcgcgctcttccggggaaacctgacaaaagtcaaatatttctgggaccagtgttcctctccccactcctacagatgacgccattgcttcatctgccgctccccacccccacccacccctgagcaatgacgtccctccctgacatcccaccccacctccagttatcctggcacaacgtttccatttatcagatctctccctcattctccgaggggtctgcagttgctggtttggtttcagagcatctctgagaaggtgtggtcagcagacaatctctcttcagcagtttgctcctcttggctgataagtgattggcagatcttatgaggtctgacaaggtgctaagacaacacacagcagaccacactaggggagattgtaacatcaactgagtcctttgcagctcagcccggatcccactccatactcccacaggggctcagtggtcatatctatgaggccggtgacctgctcaggagtttccccctcaccctacccccagatgcccactgaccaggatgcttagccactagctgcagacacacttgccggagagtaaggtgtctgcagcggcttcaactgcctttgcccccagtcttattctatttaacttgataaaggtttaccgagcagttattatgtagctcaaggggcagtgcggggagggagcaggacaccacattggcattgtcttctttgcccctcccttctcttttccattcacccacttgcccagcatccacactgactctgcccccaccttgccccttgtccctttcttctgactgttcatctctctccctgccctctcattttccttttgccattctccttatcctataactgttcaaatgacaataagggaagggattccttacaaatcctacaggagaaacattcctttaaaactgcagctaatgttatcacagagaccctgaccatggcggctgagacaacaggactgtgcttctgtctgtctctctgcacagcaaatgtaatgacctttcggtaagacccgctcttatgtccatactttgatgcttgctgaacaatagattggctcattttctgctcaggacccaagccacactgcaaacttatggcctgcgctctattcgtgatcgtatctcaacaaggagcgcttccaagtttcagagccttccttggccagccccttgggaacccaccaaaacgaacttgctgttttccagtaaggtgaaaagtcctgaaattgtttcattttcaggggtgtgtggaagtcttggggatagcctgcgggacatgccaagtgcccttacacctccggtcagtctgtgtagtcggcagatcaactctgtgcctagttcctatctttctgagccactgtattcccatctaagtggggccgagagtgcctagctgggtggtggaggtaattcacataaaagatgtaaagcatttggctgtcaggtgggagatactgtgaattccagcctcgtcactaggggcgctgtttctctcagcccacaaatttccccttgagcctaagcctttactgatagcctcttgaggagccatctggagacaccctgcctggcacagaacagtggcgcagtctttgatagattctagggtggttgtcactggagggtcacagacacagtggtgtgtgtcaaggagggtgtgagagctgcatcctcctgactcattgggtgaccttaagcaagtgacttcattcttaccttttcccatttccttttcctccaaataggaataatcatatgcagacgtggctcccagggataattgtgatgttaaatcacgctattattattcagccctggcttttcaaataaaaatgcttgctgcctctggagccctggctgacttcagtagagatgcctccgagcaggagagggggcaaaatcaaatcaaaacaaacccaactctttcaaggaactctggatcaagagggaaaacagttcctactgtgtgctggtcctttaacagacaccgtccagaggcgcagggagagagggagctggggctgtctgaggagcccaaggtttgaccacacagagaatattcaagtttcaggtgatgacccttttccttaatgggttctgccaccagcaatggctttgaacttgctggggtgcaggatgcaacctgcctctactgcaatcagcccacctggcctcctgtcaggagattgttattagatgttattagataatatgtttgcatgcctgaagacataaaaaaaataagctgaaagctatcagacttgtgtcagtcgaatgaaacaggctcagcatactttcttggtgaagggtcagacagtaacaattttggctttgtggacggcggagaccttgtttcagaagtcaaggatgctctgtggcatgtacacacttgtggacagctgtcagtggggccagggctctccagataaatggaatccatagaaacccacagaatctgtgaatgggaagaaaacatttgggccatgtgattatggagtagggtatgtcctgattacctatttctgattgttaggatgatcaaacctctttcctccactgtctttccccactgtgctcttatgtgtgtcttgagtatggccaaggacacattttgtgaagtgccaggtgaagtggattcatctaggaaggctccaacttacctccccctggctgagcaaacctcatgttgccttcccatttctgaatccattcaaggctaaaagctgctttctcctcagtttgccacagatggagacagctcacagcaggtccaacttcccagttctgtccctcagggacagctcccaatgaaaaggtggaaatctctgccccaccctgactctggagccaccaactttgctcagcaatttgtaaagcctttaaaggtgtcaaaacaaaacaaaacaaaacaaaacaaggaaaccacagctttcatcttaaagggtataagagaaagtttattctggagccattttaagtgaccgtggtctgggaacacagatggaaaatccacattctgatatgaaagcagtttcatgaagttttatcattttatggaacaaagaaagtcagaaatctaggcaagtttgagatacactggtgggtacatcagagaggcaggtgcaggaagataggggaagctttccggaggcccaagatgctatctgatgacattcttagcttttgggttggaaaccagtgtgtctggtaagacaatagattctgagggtttttttttttttaatctattagtcacaggatattagctctgacacagaggtgggcaatggatggttgttctgggagctgaagataagaaaactagctttcgtaccgttaacaatccagtctctccacagtacagaacttctcatcggttaatcagtctgtaaatacagcttcttttcaggagtgtgtgttcacagtaaggaggaaatcagatgggggattgtctcaaggccagcgtgtaaagacacattcaacctcagggacggagctgataagctgggttgtctattcacaagtcaagagaactcagaggagcaacttcctttctccaaaaagcttttacctctagggattctgagagaatctgcataagcttggtacattttaactcctgagaagcagcgattgttacaggattggtgtgcttatgaggggtaccagctcccttcccaggtagtaagccccacgcggggcagagaatgtatccatcacatgttggttctgtgtccttttgactcttttcccttcctttgtagcccacataagaccagactgcggccttcatattttcttatttaaagtgcttactgtgttccaggcatggcttagagtgtctagtgtttgtcaggcatcggcttagagcgcctagtgtgtgccaggcataggcttagagtgcctaaagcaaccctcacactctttgatagcatcccctctgtatactggagaaagccgatacatagcaaggtcggttttccccaagaggtggttaagaggccagctggaatctgtgctctcctcactaaacaagctcagggccctgcagccaggccccggcaggattggacctcgtctttctcagaatcactgagcccccttccctgccccaagctgcttctccaaattgtttgagttggccatccaagggtggcagatacaaatgtctgctaggtgctcagcaggaacagtgggggtctgtctggatgtggtccctccctggatttcctgcagccacccatgcacgtccagacagacttcttggttgtgagacctgaggctgaattctaaagagtgtgtgagagttagtcaggtcaaaaggtaatgggagtgggtgacagcagttcgaggcagagggaactctgtatgcaagggttggttgtatagcatgtgaccttggctatggaattaaaatggcttggatgtgcccaggtcaggccgagctcatatcaggatgcagtgttttgtgtatttgctaggatgctgtgaacaaatctctttgcacctccacgttctctccactacaggaatactcttggtttcagccttatggggttgtcttgaggactgtgtaaaggatggagcacagacatgcgtaatgtgcacttcacaaacacaggctctttgtgttgggagagatgctgagtatggccagaagagtcagacctttggaaggcatttcagagcctgaaggggctggcacagagctgggtagcagcacagtcagacgggagtcagcagagtgctctggctactgtgtagactgtgtctagggggatatgcttggatctgctccagaaccaggcagaggctgaaggctgcaaggtggggcaaggagatggaacggcctgggaagtcatcagtggacaagaaggcacggcacggtctgcagtagactgtctaagtgccaatctcccttggccccgggggagtctgtaatgcaggctcggtaatggcgctcatgccttaagctgctatgtatgtggttagttcaagaggctgctcagaatggcaggtacttagctacttcatcgcctatatcatcacacagaagctaagatgctggcaccagttttgtacagagatattgaaattgggtctatcatggagctcactggtatggagctctcccccaacaggttgctcaaatcttataacagattactactttgatcagcatcagaggaaaaaaaaaaagagccatatattttgtctgcatgggaatgatgcattcacctgcccggacctttggttttgctctactgatagaacgcaggagacacccaagggtcacagagacacccacagttctgtgtttttccactccgagcttccctgtgccagcagccgttgagcccagtctggaagaagcacatgagcctgcctctggcctgagctggtgggtagactaagcctggggtagggtcggtgctctgtggattgttgctgggactcatagaagatttagctgctgctgactcaaacctgttctacaaactgcaattataaggtggagagtgggcccgcaccccagcttcattcactgctcagggcaggatgcgggcagctctctgcagtaccagcgcccctgtaggggcttagaatggaggaatctcaatagcggcaactgagggaatggttaggaagctgaccgtgtaccttaagagaggatacttgtcctggggtattccagggttttggctcatggctgggatctttggagtccctgagcatcagaagaaacagagcagttctcaggaacctggagggaagatggacactgtttcttatcttggaggcagtagatcaggatgtctaggtgggaagagagtaaccccccaaggtggctgcccaggagtccccggggcatcacagctccacttgtaagaacacacagtgatttgactttgagttgtctgatgaggcggagtgatgcttcagttgttaagagtgaacccatgcttggttcccaggacccatgtcagtcacctcacaactgcctgtcactctaactctgggggctgcggggcaccttctgctggcctccaaggacacctgactttatagcatgtatcctacacccccccaccacacatagacatataattaaaaataaaataaaaccttttaaaaattgactatgaaagggacaaagttaactttttgctttaattgcaatatagttgcatcatttttcgttttcctcccccttcaagtccttttcatgtcatctccctccagcccctcccatattcaccccctcaaaagggacaactttttgtttttcgtttttattggatattttttaatttacatttcaaatgttatcccttttcccagctttccccccttctggaaaccctctatcccatcctccctccccctgcttctatgagggtgttcttcccccacccatccactcccacctccccaccctctattccccaacactggggcatctatcaagcctacataggaccaaggacctctcctcccattgatgcatgaaaggccatcctttgctacatatgcagctggagccatgtgtactccttggttggtggcttagtaaggggcaacttttaatagactttttaaaaaaatgaaccatcagctcatctactaagcatttgcaaagggcctactatgtgctagctggagttttaaatgctaggataagaatttaagtcagctaagttctgtgcctttgagagctaatgttctagttggggaaatgataaagacagataactcagctggtgggagcctgtgacatttattttatttaatgcaagaaaggaagtcttgtgaaaacctgaggtccagtacgccagacagaggaagagaatgtacaagggtcctgaggcaggaacaggctaagcctgtttgaggcccacaaacaagtactttggagagggcaagagatagtctgtgttcagagagttcgtgtgcgtgcatgcatgagtgtgtgtgtgtgtgtgtgtgtgcgcgcacgcgtgtgtattgaggggcaaagcgttcacacaactagttagggcgtgcaccattgtgatggaagaggtacctgttgctgacacgtgaaacctaatcaaggaaagacaagtaacaagaagacgggaaaggttctccaagaagaggaaacagcatgcgcaagaccaggaggtgaggcagagcctggcacagcctctgaaagtagatctcgggtctgagattggggttagagagggagccagactggtgggatgtgtgacagtgaggcagagggggaggccttactctacctaatgggcgccatctcagaattaatgtgccgaggaggatagttcatgagataatgagggaagccaaggagggaggccagcatggagaggggtgaaagtgaagcagttgtcagagtgaagggcagtgctgaagtcaaagaggagagttccaaagaccttgggaaggacagaaacagcagaggtgtatgtcaacgggacagcccttgcttctatgtccagcgtctagcctggggactcagcacatagggacagtcctggaggttggtggtcgtcacagtgtgacttgaaggtggacagaccagatcaatccctgacttggcttcctagctacgcagaaccattttcctatgcctcagtttctccacctgtcaggtgaagactatagcatagcctgcctcgcagagcagagtacgtggattgtctacagagagaggatcccagggagcagtgattactcagctctcaacctttcctgagtccttggccttgactcctaggttctctccccagcttctgtgtccttgacgacagggtgggcaggagaatgcctgcttcgtgctgctcttacaccacacacgagtaagaggtggccttgtctgcttcagacacacttcaccaccttgtccttctctctctccagcaaacaagtgagtcagaatggcgtggtccaggctgatgctggcagcttgcctcctcgtgatgccctctaatgttatggcggactgcctgtccctgtgctccctgtgtgcagtgaggattcaggatgggccccgtcccatcaaccccctggtaggtctcagcaaggtttctccatgttattctatttgtgtgcagagcccagagaggggcagttgcatatctgggcagctgtgcctctgccgcagtgcatggagtggacagccgggtaccgaggtctgaggttgtctagttcccactgcattctagacttcattaccatggctccccaaggtctgcatagaggccatgtctccaggcagccaagtgcctggcaacggggtgtagaccatggtgtgctcgctctgccatctgaagcactgggtgcacattccaaagcttcctttcctggctgacaggcacctccaaattcaggctcctggcagaagctatcaccatgacactctcagaatctgccttgtggggctggggctctcctgggcctgggcctttgctcctggcttgctgacattattttgactcgtgctttctcccacacctcacccagcagtagtgtttattagctgactgtggtggacaacactcaggctcgggttctggccctttcttgctcccttactctggaactctctcctgagtctcattttcctcatctaatgagggttgattccctgagatcatgatgattagtgtttaatatcttgactggtctgtgttcactgagctgcacacagtgtgcccatcacatagtgtgtccgtgctcactcttaggaaagcaatgtgcctaagcttggggactccagccgcagaccttcagaattcaagctcatcactggtgttgctcattactgtcaacatcattagagtcatggaggtgattgaatactctctacagaactatgacgtagatcttatacccccaacgtacaccccctcaagtgtgctgagcccttccaccccccatgcacattctttcttctcttctgaaaagctttcagtctctaaagtgttagtagtcccctccatgcctgatagaggtcggtagagcaactaggccctttgaaagggcaaaagtgtaagcgcagggcaatcggaagatcttgcttctacttttggccttgccacccgtgtctctgcgcctttgggatagctgcctaggctctgtaagtctaatttctcatttctcacatgacaaaactcttgcatcctttcagatttgctccctggagtgccaggacctggtgccgccctcagaggagtgggagacatgccggggcttctcatcttttctcaccctgacggtctctgggctccgtggcaaggatgacttggaagatgaggttgctttggaagaaggctacagtgcactagccaagctcttggaacccgtcctgaaggagctggagaaaagccgactccttaccagcgtcccagaggaaaagttcaggggtctctccagcagctttggcaacggaaaagaatctgagctggcgggtgctgaccggatgaatgatgaagccgcacaggcgggcacgctccattttaatgaggaggacttgagaaaacaggccaaacgctatggcggctttttgcgcaaataccccaagaggagttccgagatggcccgggatgaggacgggggccaggatggggatcaggtagggcatgaggacctgtacaaacgctatgggggcttcctgcggcgcattcgccccaagctgaagtgggacaaccagaagcgctatggtggtttcctgcggcgtcagttcaaggtggtgacgcggtcccaggagaaccccaatacctattctgaagatttagatgtttgatgcataacacttcccctccatggagttgggtctgcccccttccttgtactagaaccccctgcttcatcctcttgtgcttgttccgctgcttgctccattcagcactcccatgagaacgtctaagagcatcccacccctgttctgcttgttttcaggcaggggcagggaagagtggagatcccctgccaataggcccagggtccggctcaaaccccaagacctctaaactaccactactagtggctttttgacttcagcgtaaccgctctgtccctgtgatccctgcccatcccagaatccagagaaacttgttctttattcctatacgtctgtggtttccaaacttcactgaacgtgtgctcctctcggaagaaaaatgagcacttcctggtagtattgcatgggaactgtgagatatgcagtgtagaaacataaaaccagatacatgcaaatgaggatcctaactatattctcagccacagaggctcttcttatgcaccctaccctggaggccacaggataaatgaaaagccttttcaaggacagggattataaccctagagaggctgggcctctagcatgctaattgtctgtacacccagagtcctcatagtttcaaacgtttcctggaaaaacccagctcctagaccccggttagggcagtacctttcccaagggtgaaatccccttgcttatgatgagacgccatccttctgaaggctgaagtgataaggggggagcaaagcaaactaatttgttctgtacaacaaattcagaatttgggccagttcccacagccctcattaaaaccgatcagtatcacacttctagcccatgaggaactgcatcggagtggagctgatgcggctgggcacagctgtgctcttcagtagtcgagttggctgcgggagccggagccatttggctgagccatttggcccagcgtatgcactggcactgggaaggggatccagaagcggtagctgaaggagagactgtcccgcctcaaaactacagcaagtctcattctctctgtctccaaataaaagcagaggaagaagaagacaactgagtgttcaggatcaagtggattcgctggattcaagtaacccagagtcagagacaaggtcctctttggtttcacctccccatctccaggcagatatttagatgatgagttaacgggatggggctggacaggagaggaagcagactggatttgtatatttgcataagatatcttgagtgtccctctccacacacaggtcacagcctgtagagcccttcatgtgttctgaaatgtgtagattgttttacggtgacttgtgtgctgcttaaaaatcacatttatccaacctacttgggacatgtgatccttaataggtctgtgtagtttcctgcttctctggtataataaataaagttatgcggatggcttgctgtgtttgcatgaatatattcgtgtcgtctccagaaaaagtatatggctatatatatgcatatatgatatgtagatacttacacaagcacacacacatgtatagctgtattggaggattgaacctagttcctcatgcatgataggcaaacactctaccaccaagccatagctccagcccctgtagtcatattagaaaatacagtgtggaataacagaaagcaagaaagcaagtgttttacagattcacatctgtaaattgagtctgtgtaactttgggcaagtctctgatctctctggaccttacttcttatttctataaaataaagatgaatagggcagtgttagtagtatggctgagaggatcagatgaaatcaaacatgtaaagttcccagcacttagtaggttcagtaaatgagtttccttccccatatggtcttaaggaacaatggcaaagttgggcatgtggcaacaaaatagggaggacagagagaggtactgcagcctcaggggccacttcctgtgtagggaaaccgggacaagtcacagatgttcctaacaagagtaactgtgttagtggcaaacatatactgtcctctgctaaacagccattcatcactgagtggtgcatataaggtgtccttatgtgggatgtgtctgaatggctgtaaatgtgggctccagtgggacccttgcttagagtaacttcccagaaagcacag

SEQ ID NO: 6 - DNA, 7094 bp, artificial sequence
  sequence of the targetting vector from rTetR to the 3′-end of the TRE sequence including the translation start codon of the dynorphin 5′-arm
atgtctagattagataaaagtaaagtgattaacagcgcattagagctgcttaatgaggtcggaatcgaaggtttaacaacccgtaaactcgcccagaagcttggtgtagagcagcctacactgtattggcatgtaaaaaataagcgggctttgctcgacgccttagccattgagatgttagataggcaccatactcacttttgccctttaaaaggggaaagctggcaagattttttacgcaataacgctaaaagttttagatgtgctttactaagtcatcgcaatggagcaaaagtacattcagatacacggcctacagaaaaacagtatgaaactctcgaaaatcaattagcctttttatgccaacaaggtttttcactagagaacgcgttatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagggggcgcgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtcccctggcggacgggaagtatcagctcggatccgtcgacagatctttaattaagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaggcacagtcgaggctgatcagcgagctctagggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcactggggatccactagctaataacttcgtatagcatacattatacgaagttatattactagttctagagtcagcttctgatggaattagaacttggcaaaacaatactgagaatgaagtgtatgtggaacagaggctgctgatctcgttcttcaggctatgaaactgacagatttggaaaccacagtacttagaaccacaaagtggggatcaagagaaaaacaatgatcccacgagagatctatagatctatagatcatgagtgggaggaatgagctggcccttaatttggttttgcttgtttaaattatgatatccaactatgaaacattatcataaagcaatagtaaagagccttcagtaaagagcaggcatttatctaatcccaccccacccccacccccgtagctccaatccttccattcaaaatgtaggtactctgttctcacccttcttaacaaagtatgacaggaaaaacttccattttagtggacatctttattgtttaatagatcatcaatttctgcagacttacagcgcgatccctctagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatggccgatcccatattggctgcaggtcgaaaggcccggagatgaggaagaggagaacagcgcggcagacgtgcgcttttgaagcgtgcagaatgccgggcctccggaggaccttcggcgcccgccccgcccctgagcccgcccctgagcccgccgccggacccaccccttcccagcctctgagcccagaaagcgaaggagcaaagctgctattggccgctgccccaaaggccgacccgcttccattgctcaggcggtgctgtccatctgcgcgagactagtgagagacgtgctacgtccatttgtcacgtcctgcacgacgcgagctgcggcgcgggggggaacttcctgactaggggaggagtagaaggtggcgccaaggggccaccaaagaacggagccggttggcgcctaccggtggatgtggaatgtgtgcgagccagaggccacttgtgtagcgccaagtgcccagcggggctgctaaagcgcatgctccagactgccttgggaaaagcgcctcccctacccggtagaattggccgcgaagttcctatactttctagagaataggaacttcggccgccaccgcattaacgtttacaatttcgcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaagcttggtcgagctgatacttcccgtccgccaggggacatgccggcgatgctgaaggtcgcgcgcattcccgatgaagaggccggttaccgcctgttgacctggtgggacgggcagggcgccgcccgagtcttgcctcggcggcgggcgctctgctcatggagcgcgcgtccggggaccttgcacagatagcgtggtccggccagacgacgaggcttgcaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcctctagaattcctgcagcccgggggatccactagctaataacttcgtatagcatacattatacgaagttatattactagtatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagttacccccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacgaacctggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcctgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacaccttcctcttcttcttgggcatggtcgagggtcccgcggaggctggatcggtcccggtgtcttctatggaggtcaaaacagcgtggatggcgtctccaggcgatctgacggttcactaaacgagctctgcttatataggcctcccaccgtacacgcctactcgacccgggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacgcgttgggagctctccca

SEQ ID NO: 7 - DNA, 14634 bp, artificial sequence
  sequence of a dynorphin gene targetting construct
gaattctaaagagtgtgtgagagttagtcaggtcaaaaggtaatgggagtgggtgacagcagttcgaggcagagggaactctgtatgcaagggttggttgtatagcatgtgaccttggctatggaattaaaatggcttggatgtgcccaggtcaggccgagctcatatcaggatgcagtgttttgtgtatttgctaggatgctgtgaacaaatctctttgcacctccacgttctctccactacaggaatactcttggtttcagccttatggggttgtcttgaggactgtgtaaaggatggagcacagacatgcgtaatgtgcacttcacaaacacaggctctttgtgttgggagagatgctgagtatggccagaagagtcagacctttggaaggcatttcagagcctgaaggggctggcacagagctgggtagcagcacagtcagacgggagtcagcagagtgctctggctactgtgtagactgtgtctagggggatatgcttggatctgctccagaaccaggcagaggctgaaggctgcaaggtggggcaaggagatggaacggcctgggaagtcatcagtggacaagaaggcacggcacggtctgcagtagactgtctaagtgccaatctcccttggccccgggggagtctgtaatgcaggctcggtaatggcgctcatgccttaagctgctatgtatgtggttagttcaagaggctgctcagaatggcaggtacttagctacttcatcgcctatatcatcacacagaagctaagatgctggcaccagttttgtacagagatattgaaattgggtctatcatggagctcactggtatggagctctcccccaacaggttgctcaaatcttataacagattactactttgatcagcatcagaggaaaaaaaaaaagagccatatattttgtctgcatgggaatgatgcattcacctgcccggacctttggttttgctctactgatagaacgcaggagacacccaagggtcacagagacacccacagttctgtgtttttccactccgagcttccctgtgccagcagccgttgagcccagtctggaagaagcacatgagcctgcctctggcctgagctggtgggtagactaagcctggggtagggtcggtgctctgtggattgttgctgggactcatagaagatttagctgctgctgactcaaacctgttctacaaactgcaattataaggtggagagtgggcccgcaccccagcttcattcactgctcagggcaggatgcgggcagctctctgcagtaccagcgcccctgtaggggcttagaatggaggaatctcaatagcggcaactgagggaatggttaggaagctgaccgtgtaccttaagagaggatacttgtcctggggtattccagggttttggctcatggctgggatctttggagtccctgagcatcagaagaaacagagcagttctcaggaacctggagggaagatggacactgtttcttatcttggaggcagtagatcaggatgtctaggtgggaagagagtaaccccccaaggtggctgcccaggagtccccggggcatcacagctccacttgtaagaacacacagtgatttgactttgagttgtctgatgaggcggagtgatgcttcagttgttaagagtgaacccatgcttggttcccaggacccatgtcagtcacctcacaactgcctgtcactctaactctgggggctgcggggcaccttctgctggcctccaaggacacctgactttatagcatgtatcctacacccccccaccacacatagacatataattaaaaataaaataaaaccttttaaaaattgactatgaaagggacaaagttaactttttgctttaattgcaatatagttgcatcatttttcgttttcctcccccttcaagtccttttcatgtcatctccctccagcccctcccatattcaccccctcaaaagggacaactttttgtttttcgtttttattggatattttttaatttacatttcaaatgttatcccttttcccagctttccccccttctggaaaccctctatcccatcctccctccccctgcttctatgagggtgttcttcccccacccatccactcccacctccccaccctctattccccaacactggggcatctatcaagcctacataggaccaaggacctctcctcccattgatgcatgaaaggccatcctttgctacatatgcagctggagccatgtgtactccttggttggtggcttagtaaggggcaacttttaatagactttttaaaaaaatgaaccatcagctcatctactaagcatttgcaaagggcctactatgtgctagctggagttttaaatgctaggataagaatttaagtcagctaagttctgtgcctttgagagctaatgttctagttggggaaatgataaagacagataactcagctggtgggagcctgtgacatttattttatttaatgcaagaaaggaagtcttgtgaaaacctgaggtccagtacgccagacagaggaagagaatgtacaagggtcctgaggcaggaacaggctaagcctgtttgaggcccacaaacaagtactttggagagggcaagagatagtctgtgttcagagagttcgtgtgcgtgcatgcatgagtgtgtgtgtgtgtgtgtgtgtgcgcgcacgcgtgtgtattgaggggcaaagcgttcacacaactagttagggcgtgcaccattgtgatggaagaggtacctgttgctgacacgtgaaacctaatcaaggaaagacaagtaacaagaagacgggaaaggttctccaagaagaggaaacagcatgcgcaagaccaggaggtgaggcagagcctggcacagcctctgaaagtagatctcgggtctgagattggggttagagagggagccagactggtgggatgtgtgacagtgaggcagagggggaggccttactctacctaatgggcgccatctcagaattaatgtgccgaggaggatagttcatgagataatgagggaagccaaggagggaggccagcatggagaggggtgaaagtgaagcagttgtcagagtgaagggcagtgctgaagtcaaagaggagagttccaaagaccttgggaaggacagaaacagcagaggtgtatgtcaacgggacagcccttgcttctatgtccagcgtctagcctggggactcagcacatagggacagtcctggaggttggtggtcgtcacagtgtgacttgaaggtggacagaccagatcaatccctgacttggcttcctagctacgcagaaccattttcctatgcctcagtttctccacctgtcaggtgaagactatagcatagcctgcctcgcagagcagagtacgtggattgtctacagagagaggatcccagggagcagtgattactcagctctcaacctttcctgagtccttggccttgactcctaggttctctccccagcttctgtgtccttgacgacagggtgggcaggagaatgcctgcttcgtgctgctcttacaccacacacgagtaagaggtggccttgtctgcttcagacacacttcaccaccttgtccttctctctctccagcaaacaagtgagtcagaatgtctagattagataaaagtaaagtgattaacagcgcattagagctgcttaatgaggtcggaatcgaaggtttaacaacccgtaaactcgcccagaagcttggtgtagagcagcctacactgtattggcatgtaaaaaataagcgggctttgctcgacgccttagccattgagatgttagataggcaccatactcacttttgccctttaaaaggggaaagctggcaagattttttacgcaataacgctaaaagttttagatgtgctttactaagtcatcgcaatggagcaaaagtacattcagatacacggcctacagaaaaacagtatgaaactctcgaaaatcaattagcctttttatgccaacaaggtttttcactagagaacgcgttatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagggggcgcgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtcccctggcggacgggaagtatcagctcggatccgtcgacagatctttaattaagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaggcacagtcgaggctgatcagcgagctctagggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcactggggatccactagctaataacttcgtatagcatacattatacgaagttatattactagttctagagtcagcttctgatggaattagaacttggcaaaacaatactgagaatgaagtgtatgtggaacagaggctgctgatctcgttcttcaggctatgaaactgacagatttggaaaccacagtacttagaaccacaaagtggggatcaagagaaaaacaatgatcccacgagagatctatagatctatagatcatgagtgggaggaatgagctggcccttaatttggttttgcttgtttaaattatgatatccaactatgaaacattatcataaagcaatagtaaagagccttcagtaaagagcaggcatttatctaatcccaccccacccccacccccgtagctccaatccttccattcaaaatgtaggtactctgttctcacccttcttaacaaagtatgacaggaaaaacttccattttagtggacatctttattgtttaatagatcatcaatttctgcagacttacagcgcgatccctctagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatggccgatcccatattggctgcaggtcgaaaggcccggagatgaggaagaggagaacagcgcggcagacgtgcgcttttgaagcgtgcagaatgccgggcctccggaggaccttcggcgcccgccccgcccctgagcccgcccctgagcccgccgccggacccaccccttcccagcctctgagcccagaaagcgaaggagcaaagctgctattggccgctgccccaaaggccgacccgcttccattgctcaggcggtgctgtccatctgcgcgagactagtgagagacgtgctacgtccatttgtcacgtcctgcacgacgcgagctgcggcgcgggggggaacttcctgactaggggaggagtagaaggtggcgccaaggggccaccaaagaacggagccggttggcgcctaccggtggatgtggaatgtgtgcgagccagaggccacttgtgtagcgccaagtgcccagcggggctgctaaagcgcatgctccagactgccttgggaaaagcgcctcccctacccggtagaattggccgcgaagttcctatactttctagagaataggaacttcggccgccaccgcattaacgtttacaatttcgcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaagcttggtcgagctgatacttcccgtccgccaggggacatgccggcgatgctgaaggtcgcgcgcattcccgatgaagaggccggttaccgcctgttgacctggtgggacgggcagggcgccgcccgagtcttgcctcggcggcgggcgctctgctcatggagcgcgcgtccggggaccttgcacagatagcgtggtccggccagacgacgaggcttgcaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcctctagaattcctgcagcccgggggatccactagctaataacttcgtatagcatacattatacgaagttatattactagtatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagttacccccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacgaacctggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcctgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacaccttcctcttcttcttgggcatggtcgagggtcccgcggaggctggatcggtcccggtgtcttctatggaggtcaaaacagcgtggatggcgtctccaggcgatctgacggttcactaaacgagctctgcttatataggcctcccaccgtacacgcctactcgacccgggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacgcgttgggagctctcccatatgtagcaaatgtgcagcttgcttttcatgtgagatccccaacaattggagcaggcttaccctgattctgttgcctgactgtgaatcctgttcccctaactgggctgcctcatctggtcttagtagtctagtctcagtgggagaggatatgcctagtcctgtagtgacttgatgaaccagagagggttggtacccagggagggcctcccccttctcagaggagaagaggaggtaggaagaggggatgtgtgaggaggaacaggaaggagggggggatgtgattggatgtaaagtgaataaataaataagttaatgaaaagaattcatcctatacgttatggattccacaatactactgttccagcttccattttctaggttaccttcaatggttaccaagaggcatgcctatgaagggtggacaacacggctctaggggcacagggtattttgggctcagattttcattctcttgagtggagaggagaatactgactttactgttcagagacaaaatctgggagcatcaagtaagtctacagtatacctgacttgccttaagccatgtgtgctacatgctgttatgttccgagagtctaggcccgtggcccatgtactggtctagctctgtggatcagccacagcagggtactggagctcctcagaacccggtttctggtggtgactctgatcacggtatgtctgataccacccgagtaaggcacttgcctgttgaaagaacaacattctactgttggtatgaaagcagtggcattaagtaattttcctggggttgtaacctatgtgctatgagagcccctcaagtgtggatacttagaacatttccacaccccagaggactcaacaagatctgttttaatgagctatttgtggggacagagactatcacagcccttccagattcctgaacttctgtccctgcccaaagccactgtgcattggtcacagagcagacaggcacaggcgaccaacataccaacaccctggtaacctttaaaaagcccattactaataagcaagcagtttctagacaacatcgaatacagtctctagataggaaatttgttcataatgatcatgacagtcattaccattctctgctttgggtcacccctcgaagtccctcataggaaccaacctagcgatggggcatatctattaattaaaggctccaaagtgagacaacaaaaatccccacctcagccttgggaacaactcttccctaatagatgtcagtttgacatccataacgctacacaggagtgtgcagatttataacaaagatttgtaattacttctccaatgtgtaaatgtgcgcccatattgctctcacccatattgctctcacgcacaatttgttatgtgttatgtctaatttcagttgaatccattatttgttctcgggagcctttattactgctgacaggaagtgtgttttctgaaagggaggacctctctagctctccaatgcttccatacccctgccaggcccctgcaggagctgccctgaggggatatttctgataattgacaagaggagtgaaatattgagatttgagaaagggagactggacagagtccaggcccatgggcctgagtattgttctagctctgcacagcagctgagacaggggactggacctcttcagtttccattaaatgctgctgataacatttccagctttcaggagcctgcacatgatgattggtatctgtcaggaaattctgcccaaagccactgagtttttttttttcgttgcatcctatgctatatgcatgaaggagatggcttccaccctatgatttgagaaagttctcagacacgcaggacaccatccagctaaaagttgttgcataaccagagcagtgtcttccctacctgaattcacctcatttctgatgaccactattgagcatctactgtatactcagcaacaggcaagaggcaagggatccattggagcttaagcaaagtactagttcacagggttcaagtctcaaggagggataaagtatctacaaaagaatagatggatctgacaaggatggggtgagatggttgtgagttctagggaggaaaaggaagggaggagggagtgagggatgggggaagggtagccacttagagaggtcacaatggtacctctgaaagggtggcatgagaccagagatgtataaggttggcattaagttgtagttgtctagatgtgtgtttgtgtgtgttttccacgcacagagaatggcaaattcaaacgctaacaggagagtgtgtgattatttgtggtagagaagcaagcaggaggggccgagggagggcaggggttaaaaatggagcagaagtcaatgggagcagatggcattgggcttctgccctcttctacctcatactgttacccatgacagcctccaagggtcctgctcaggatagtttatgacactgtcgctcaactgatggaccctggaaaagacccttgacccatgggggataagagcttctagaaggacgtaatctaggtcaggtcaattggcatcgccaaataatgatgatggaaggttctggaagtaacccagaagtaacttttctttcctggtatcaaaggcccagtcataataatttctccaaaacaggcagccaagggaattgggacttggggttcacggggtggttcaaattcctcacaggcagggggtagctctggaaatgctgcaggtgggagctgggaagccagtttgcaaaaaatcagtagcacttgagaaattatcctgcagaagacacatagagctcctacctccccatccttcttagcctgagaaatgtagcctcatgctttgagcttttatgaagaaaattggtctctgagttgctttatataaaaaagattcatcattgtcaagtatatacatatcatatttttttgtgaatcaaattggatttcagtgcattggagactattgctttcagtttctttcttttttttttttttttttttgattttgcaggttatactacttcttgcccgtttataagcatctgaaaaccatcaagaatgttcatgtgaaattaatcaacagtcgggattgagacaaggagttttcactaaccccttccctggggttagtgtatgacccctgaactcaggactgtgtagtccagctggggagagtccttagagcagtggttctcaacctgtgggtgacaagcctctagctccagaaatatctacacaataattcataacagaggcgaaattacagttatgaagtagcaacaaatgtaattttatggttggtggtcaccacagcatgaggaactatgttaaggagtcacagcattaggaaggttgagaaccactggcttggagcaaatgccagtagaaaaaaacatggagagttattctgaccatgcagaggagagagaggagctttgggcacagagagcagaggcctgggtccaaatgaaggcttgaggcggggcagatagctgaagtgctacagggacatttcagagacaggaagagcttgatcatgtaagccctgtaggtggccagtgggcctcatctcagaaatgaagaaagcaagtaaaagatctaatcaaagaagcggcatggccagggtgctgattggagcaacactgtgcaccagagcaatgttataccaagggtttaaaacctcacactgctagggccattcttctggccatgccatgaatgctgctggacgctacaccattgctgtcaactgatgccccacatcattcattcactgctaccccaggtctcacatttaaggcgagacagggctaaagatgactcggtggttaagagtgcttgccgcacagacatgaagacctgagctcgaattc

SEQ ID NO: 8 - DNA, 42 bp, artificial sequence
  mouse NPY antisense probe
gagggtcagtccacacagccccattcgcttgttacctagcat

SEQ ID NO: 9 - DNA, 42 bp, artificial sequence
  mouse preprodynorphin antisense probe
ttcaggacgggttccaagagcttggcatgtgcactgatgcct